(19) United States
(12) Patent Application Publication
(10) Pub. No.: US 2011/0059043 A1
(43) Pub. Date: Mar. 10, 2011

(54) CHEMICAL COMPOUNDS

(75) Inventors: Michael Christopher Stratton BARNES, London (GB); Helena Jade DENNISON, London (GB); Stephen Sean FLACK, London (GB); James Andrew LUMLEY, London (GB); Pui Shan PANG, London (GB); Keith Charles SPENCER, London (GB)

(73) Assignee: ARROW THERAPEUTICS LIMITED, London (GB)

(21) Appl. No.: 12/874,512

(22) Filed: Sep. 2, 2010

Related U.S. Application Data
(60) Provisional application No. 61/239,476, filed on Sep. 3, 2009.

(51) Int. Cl. A61K 31/5513; C07D 243/24; C07D 243/26; C07D 405/12; C07D 401/12; C07D 413/10; C07D 413/14; A61K 38/21; A61K 31/7056; A61P 31/14

(52) U.S. Cl. 424/85.4; 540/509; 514/221; 514/43

(57) ABSTRACT

The invention concerns benzodiazepine derivatives of Formula I:

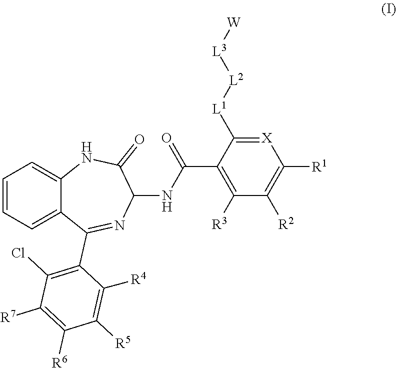

wherein W, X, L¹, L², L³, R¹, R², R³, R⁴, R⁵, R⁶ and R⁷ are as defined in the description. The present invention also relates to processes for the preparation of such compounds, pharmaceutical compositions containing them and their use in the treatment or prophylaxis of hepatitis C virus infection.

This application claims the benefit under 35 U.S.C. §119(e) of Application No. 61/239476 filed 3 Sep. 2009.

### INTRODUCTION

The present invention relates to a series of benzodiazepine derivatives and, in particular, it relates to a series of benzodiazepine derivatives which are inhibitors of the hepatitis C virus (HCV) Polymerase enzyme and are therefore active against HCV infection. This invention also relates to methods for the preparation of such benzodiazepine derivatives and novel intermediates in the preparation thereof, to pharmaceutical compositions containing such benzodiazepine derivatives, to the use of such benzodiazepine derivatives in the preparation of medicines and to the use of such benzodiazepine derivatives in the treatment of HCV infection.

### BACKGROUND

Hepatitis C virus is a member of the Flaviviridae family of viruses and HCV infection is the leading cause of chronic liver disease worldwide. An estimated 170 million people are infected with HCV worldwide. Following the initial acute infection, a majority of infected individuals develop chronic hepatitis, which can progress to liver fibrosis, cirrhosis, end-stage liver disease and hepatocellular carcinoma. Liver cirrhosis due to HCV infection is the principal cause of liver transplantation.

There are six major HCV genotypes and more than 50 subtypes, with HCV type 1 being the predominant genotype in the US and Europe. HCV has a positive-sense, single-stranded RNA genome that encodes a single polyproptein which undergoes posttranslational cleavage to provide ten viral proteins, including viral structural proteins (envelope glycoproteins E1 and E2, and the core nucleocapsid protein), non-structural proteins (helicase, polymerase and protease) and other proteins of unknown function. Replication of the viral genome is mediated by the RNA-dependent RNA polymerase.

The current standard of care for HCV infection is treatment with interferon-alpha in combination with ribavirin. However, such therapy is only partially effective and may cause significant, undesirable side effects.

An alternative strategy for the treatment of HCV infection is the targeting of HCV polymerase with small molecular weight inhibitors. For example, WO 07/034127 discloses a series of benzodiazepine derivatives that are inhibitors of the HCV polymerase. Nonetheless, there is still a requirement for alternative HCV polymerase inhibitors which differ by virtue of their chemical structure and may have superior potency against HCV Polymerase and/or advantageous physical properties and/or favourable toxicity profiles and/or favourable metabolic profiles in comparison with other known HCV Polymerase inhibitors.

### DESCRIPTION OF THE INVENTION

A further series of HCV Polymerase inhibitors is described herein. According to a first aspect of the present invention there is therefore provided a compound of Formula (I), or a pharmaceutically acceptable salt thereof:

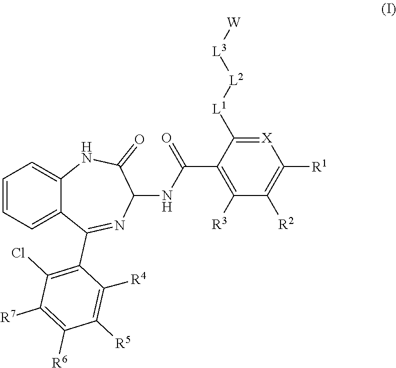

wherein:
L¹ represents O or NR⁸, wherein R⁸ represents hydrogen, C₁₋₃alkyl, acetyl, is trifluoromethyl or trifluoromethylcarbonyl;
L² represents C₁₋₆alkylene;
L³ represents O, NR⁹ or S(O)ₙ, wherein R⁹ represents hydrogen or C₁₋₃alkyl and n represents 0, 1 or 2;
W represents
   C₁₋₆alkyl, C₁₋₃alkylaminoC₁₋₆alkyl, diC₁₋₃alkylaminoC₁₋₆alkyl, C₁₋₆alkoxyC₁₋₆alkyl, C₂₋₄alkanoyl, C₁₋₄alkylsulfonyl, C₁₋₄alkylaminocarbonyl, diC₁₋₄alkylaminocarbonyl, haloC₁₋₃ alkyl or C₃₋₆cycloalkylC₁₋₃alkyl;
   aryl, wherein said aryl ring is optionally substituted with 1, 2 or 3 substituents selected from R10;
   a 5 or 6 membered monocyclic heteroaryl ring which comprises 1, 2, 3 or 4 heteroatoms independently selected from O, N or S, wherein said heteroaryl ring is optionally substituted with 1, 2 or 3 substituents selected from R¹⁰; or
   W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
X represents CH or N;
R¹ represents hydrogen or fluoro;
R² represents hydrogen, halo, C₁₋₃alkyl, C₁₋₃alkoxy, formyl, C₂₋₃ alkanoyl, trifluoromethyl or trifluoromethoxy;
R³ represents hydrogen, C₁₋₃alkyl or halo;
R⁴ represents hydrogen, halo, C₁₋₃alkyl, C₁₋₃alkoxy, haloC₁₋₃alkyl, C₁₋₆alkoxyC₁₋₆alkyl or —(CH₂)ₚ—NR¹¹R¹², wherein p represents 1 or 2 and R¹¹ and R¹² independently represent hydrogen or C₁₋₃ alkyl, or R¹¹ and R¹² are joined so as to form a 4, 5, 6 or 7 membered heterocyclic ring which optionally comprises, in addition to the nitrogen atom to which R¹¹ and R¹² are attached, 1 or 2 further heteroatoms independently selected from O, N or S, and wherein said heterocyclic ring is optionally substituted with 1, 2 or 3 substituents selected from R¹⁰;
R⁵ represents hydrogen, C₁₋₃alkyl or halo;
R⁶ represents hydrogen, halo, C₁₋₃alkyl, C₁₋₃alkoxy, haloC₁₋₃alkyl or haloC₁₋₃alkoxy;
R⁷ represents hydrogen, C₁₋₃alkyl, C₁₋₃alkoxy, halo, trifluoromethyl or trifluoromethoxy; and
R¹⁰ represents C₁₋₃ alkyl or halo.

According to another aspect of the present invention there is therefore provided a compound of Formula (I), or a pharmaceutically acceptable salt thereof, wherein:
L¹ L represents O or NR⁸, wherein R⁸ represents hydrogen, C₁₋₃alkyl, acetyl, trifluoromethyl or trifluoromethylcarbonyl;
L² represents C₁₋₆alkylene;
L³ represents O, NR⁹ or S(O)ₙ, wherein R⁹ represents hydrogen or C₁₋₃alkyl and n represents 0, 1 or 2;
W represents
   C₁₋₆alkyl, diC₁₋₃alkylaminoC₁₋₆alkyl, C₁₋₆alkoxyC₁₋₆alkyl, C₂₋₄alkanoyl, C₁₋₄alkylsulfonyl, C₁₋₄alkylaminocarbonyl, haloC₁₋₃ alkyl or C₃₋₆cycloalkylC₁₋₃ alkyl;
   aryl, wherein said aryl ring is optionally substituted with 1, 2 or 3 substituents selected from R¹⁰;
   a 5 or 6 membered monocyclic heteroaryl ring which comprises 1, 2, 3 or 4 heteroatoms independently selected from O, N or S, wherein said heteroaryl ring is optionally substituted with 1, 2 or 3 substituents selected from R¹⁰; or
   W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
X represents CH or N;
R¹ represents hydrogen or fluoro;

R² represents hydrogen, halo, C₁₋₃ alkyl, C₁₋₃alkoxy, formyl, C₂₋₃ alkanoyl, trifluoromethyl or trifluoromethoxy;
R³ represents hydrogen, C₁₋₃alkyl or halo;
R⁴ represents hydrogen, halo, C₁₋₃alkyl, C₁₋₃alkoxy, haloC₁₋₃alkyl, C₁₋₆alkoxyC₁₋₆alkyl or —(CH₂)ₚ—NR¹¹R¹², wherein p represents 1 or 2 and R¹¹ and R¹² independently represent hydrogen or C₁₋₃ alkyl, or R¹¹ and R¹² are joined so as to form a 4, 5, 6 or 7 membered heterocyclic ring which optionally comprises, in addition to the nitrogen atom to which R¹¹ and R¹² are attached, 1 or 2 further heteroatoms independently selected from O, N or S, and wherein said heterocyclic ring is optionally substituted with 1, 2 or 3 substituents selected from R¹⁰;
R⁵ represents hydrogen, C₁₋₃alkyl or halo;
R⁶ represents hydrogen, halo, C₁₋₃alkyl, C₁₋₃alkoxy, haloC₁₋₃alkyl or haloC₁₋₃alkoxy;
R⁷ represents hydrogen, C₁₋₃alkyl, C₁₋₃alkoxy, halo, trifluoromethyl or trifluoromethoxy; and
R¹⁰ represents C₁₋₃alkyl or halo.

The term “halo” is used herein to denote fluoro, chloro, bromo and iodo.

The term “C₁₋₆alkyl” is intended to mean a monovalent saturated carbon chain radical of 1 to 6 carbon atoms in length which may be straight-chained or branched. However, references to individual alkyl groups such as “propyl” are specific for the straight chain version only and references to individual branched-chain alkyl groups such as tert-butyl are specific for the branched chain version only. For example, “C₁₋₆alkyl” includes, but is not limited to, methyl, ethyl, propyl, isopropyl, butyl, tert-butyl, pentyl, tent-pentyl, hexyl and isohexyl. The term “C₁₋₃alkyl” is to be construed accordingly.

The term “C₁₋₆alkylene” is intended to mean a divalent saturated carbon chain radical of 1 to 6 carbon atoms in length which may be straight-chained or branched. For example, “C₁₋₆alkylene” includes, but is not limited to, methylene, ethylene, 2,2-dimethyl-ethylene, propylene, 2-methylpropylene, butylene and pentylene.

The term “C₁₋₃ alkoxy” is intended to mean a saturated carbon chain of 1 to 3 carbon atoms in length, which may be straight-chained or branched, linked to oxygen. For example, “C₁₋₃ alkoxy” includes methoxy, ethoxy, propoxy and isopropoxy.

The term “C₁₋₆alkoxyC₁₋₆alkyl” is intended to mean a saturated carbon chain of 1 to 6 carbon atoms in length, which may be straight-chained or branched, linked via oxygen to another saturated carbon chain of 1 to 6 carbon atoms in length, which may be straight-chained or branched. For example, “C₁₋₆alkoxyC₁₋₆alkyl” includes, but is not limited to, methoxyethyl, methoxypropyl, ethoxypropyl, propoxyethyl and butoxypropyl.

The term “diC₁₋₃alkylaminoC₁₋₆alkyl” is intended to mean a tertiary amino group which is substituted by two alkyl groups of 1 to 3 carbon atoms in length, wherein said alkyl groups may be straight-chained or branched, and which is linked to a saturated carbon chain of 1 to 6 carbon atoms in length which may also be straight-chained or branched. For example, “di-C₁₋₃alkylaminoC₁₋₆alkyl” includes, but is not limited to, dimethylaminoethyl, dimethylaminomethyl, diethylaminoethyl, dipropylaminoethyl and dimethylaminopropyl.

The term “C₁₋₃alkylaminoC₁₋₆alkyl” is intended to mean a secondary amino group which is substituted by one alkyl group of 1 to 3 carbon atoms in length, wherein said alkyl group may be straight-chained or branched, and which is linked to a saturated carbon chain of 1 to 6 carbon atoms in length which may also be straight-chained or branched. For example, “C₁₋₃alkylaminoC₁₋₆alkyl” includes, but is not limited to, methylaminoethyl, methylaminomethyl, ethylaminoethyl, propylaminoethyl and methylaminopropyl.

The term “C₂₋₄alkanoyl” is intended to mean a saturated carbon chain of 1 to 3 carbon atoms in length, which may be straight-chained or branched, linked to carbonyl. For example, “C₂₋₄alkanoyl” includes acetyl, propanoyl, butanoyl and 2-methylpropanoyl. The term “C2_{—}3alkanoyl” is to be construed accordingly.

The term “C₁₋₄alkylsulfonyl” is intended to mean a saturated carbon chain of 1 to 4 carbon atoms in length, which may be straight-chained or branched, linked to sulfur doioxide. For example, “C₁₋₄alkylsulfonyl” includes, but is not limited to, methylsulfonyl, ethylsulfonyl, propylsulfonyl, isopropylsulfonyl, butylsulfonyl, isobutylsulfonyl and tert-butylsulfonyl.

The term “C₁₋₄alkylaminocarbonyl” is intended to mean a saturated carbon chain of 1 to 4 carbon atoms in length, which may be straight-chained or branched, linked to a secondary amino group which is in turn linked to a carbonyl group. For example, “C₁₋₄alkylaminocarbonyl” includes, but is not limited to, methylaminocarbonyl, ethylaminocarbonyl, propylaminocarbonyl and butylaminocarbonyl.

The term “diC₁₋₄alkylaminocarbonyl” is intended to mean two saturated carbon chains of 1 to 4 carbon atoms in length, which may be straight-chained or branched, each linked to a tertiary amino group which is in turn linked to a carbonyl group. For example, “diC₁₋₄alkylaminocarbonyl” includes, but is not limited to, dimethylaminocarbonyl, diethylaminocarbonyl, dipropylaminocarbonyl and dibutylaminocarbonyl.

The term “haloC₁₋₃alkyl” is intended to mean a saturated carbon chain of 1 to 3 carbon atoms in length, which may be straight-chained or branched, wherein at least one of is the hydrogen atoms has been replaced by a halo atom. For example, “haloC₁₋₃alkyl” includes, but is not limited to, difluoromethyl, trifluoromethyl, chloro(difluoro)methyl, difluoroethyl and difluoropropyl.

The term “haloC ₁₋₃alkoxy” is intended to mean a saturated carbon chain of 1 to 3 carbon atoms in length, which may be straight-chained or branched, wherein at least one of the hydrogen atoms has been replaced by a halo atom, linked to oxygen. For example, “haloC₁₋₃alkoxy” includes, but is not limited to, difluoromethoxy, trifluoromethoxy, chloro(difluoro)methoxy, difluoroethoxy and difluoropropoxy.

The term “C₃₋₆cycloalkylC ₁₋₃alkyl” is intended to mean a saturated 3 to 6 membered monocyclic carbon ring linked to a saturated carbon chain of 1 to 3 carbon atoms in length which may be straight-chained or branched. For example “C₃₋₆cycloalkyllC₁₋₃alkyl” includes, but is not limited to, cyclopropylmethyl, cyclobutylethyl, cyclopentylpropyl and cyclohexylethyl.

The term “aryl” is intended to mean phenyl or naphthyl.

Unless stated otherwise, the term “monocyclic heteroaryl ring” is intended to mean a 5 or 6 membered, totally unsaturated and/or aromatic monocyclic ring which comprises 1, 2, 3 or 4 heteroatoms independently selected from nitrogen, oxygen or sulfur, linked via ring carbon atoms or ring nitrogen atoms where a bond from a nitrogen is possible, for example no bond is possible to the nitrogen of a pyridine ring, but a bond is possible through the 1-nitrogen of a pyrazole ring. Examples of 5 or 6 membered heteroaryl rings include, but are not limited to, pyrrolyl, furanyl, imidazolyl, triazolyl, tetrazolyl, pyrazinyl, pyrazolyl, pyrimidinyl, pyridazinyl, pyridinyl, pyrrolyl, isoxazolyl, oxazolyl, 1,2,4 oxadiazolyl, isothiazolyl, thiazolyl, thiadiazolyl, 1,2,4-triazolyl and thiophenyl.

Unless stated otherwise, the term “heterocyclic ring” is intended to mean a 4, 5, 6 or 7 membered fully saturated or partially saturated monocyclic ring which comprises 1, 2 or 3 heteroatoms selected from nitrogen, oxygen or sulfur linked via ring carbon atoms or ring nitrogen atoms. Examples of 4, 5, 6 or 7 membered heterocyclic rings include, but are not limited to, azetidinyl, tetrahydrofuranyl, dihydropyranyl, tetrahydropyranyl, pyrrolinyl, pyrrolidinyl, thiazolidinyl, morpholinyl, oxetanyl, piperidinyl, piperazinyl, dihydropyridinyl, dihydropyrimidinyl and azepanyl.

It is to be understood that, insofar as compounds of Formula (I) defined above exist in optically active or racemic forms by virtue of the asymmetric carbon atom, the invention includes in its definition any such optically active or racemic form which possesses the property of HCV Polymerase inhibitory activity. The synthesis of optically active forms may be carried out by standard techniques of organic chemistry well known in the art, for example by synthesis from optically active starting materials or by resolution of a racemic form. Racemic compounds and racemic intermediates thereof are drawn herein as flat structures whereas stereospecific compounds and stereospecific intermediates thereof are drawn with the appropriate stereochemistry indicated.

In one embodiment of the invention, the compound of Formula (I) has the configuration shown in Formula (IA):

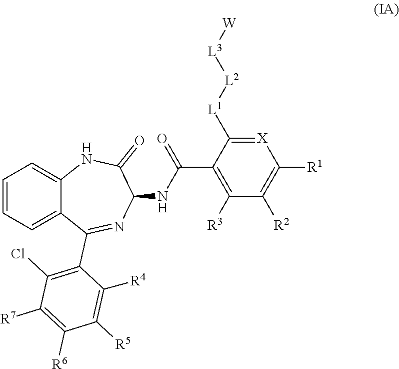

wherein L¹, L², L³, W, X, R¹, R², R³, R⁴R⁵, R⁶ and R⁷ are as defined hereinbefore.

In one embodiment of the invention, the compound of Formula (I) has the configuration shown in Formula (IB):

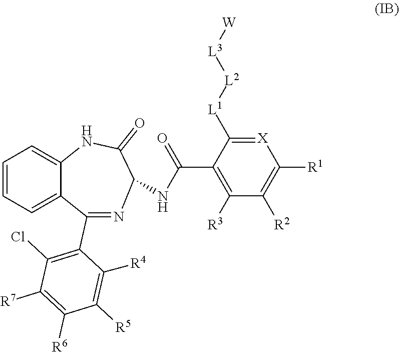

wherein L¹, L², L³, W, X, R¹, R², R³, R⁴R⁵, R⁶ and R⁷ are as defined hereinbefore.

Reference herein to a compound of Formula (I) should be understood to refer equally to a compound of Formula (I), (IA) or (IB).

It is to be understood that certain compounds of Formula (I) above may exist in unsolvated forms as well as solvated forms, such as, for example, hydrated forms. It is to be understood that the present invention encompasses all such solvated forms that possess HCV Polymerase inhibitory activity.

It is also to be understood that certain compounds of Formula (I) may exist in crystalline form and exhibit polymorphism. The present invention encompasses all such polymorphic forms which possess HCV Polymerase inhibitory activity.

In further embodiments of the first aspect of the present invention, each of the following definitions of L¹, L², L³, W, X, R¹, R², R³, R⁴R⁵, R⁶, R⁷, R⁸ and R⁹ in paragraphs (1) to (35) hereinafter may be used individually or in combination with one or more of the other following definitions to limit the broadest definitions of Formulas (I), (IA) or (IB). For example, a skilled person would understand that paragraphs (1), (4), (5) and (9) could be combined to provide a compound of Formula (I), or a pharmaceutically acceptable salt thereof, wherein L¹ represents O, L² represents ethylene, L³ represents O is and W represents C₁₋₆alkyl. Similarly, paragraphs (16), (19), (22), (25), (26), (27), (29) and (31) could be combined to provide a compound of Formula (I), or a pharmaceutically acceptable salt thereof, wherein R¹ represents hydrogen, R² represents fluoro, R³ represents hydrogen, R⁴ represents chloro, methoxy or ethoxy, R⁵ represents hydrogen and R⁶ represents chloro. Likewise, paragraphs (1), (4), (5), (9), (16), (19), (22), (25), (26), (27), (29) and (31) could be combined. Likewise, paragraphs (16), (40), (41), (42), (29), (43), (32), (36), (3), (37), (38) and (14) could be combined. Likewise, paragraphs (16), (40), (41), (42), (29), (43), (32), (36), (3), (37), (39) and (14) could be combined. Likewise, paragraphs (16), (40), (41), (42), (29), (43), (32), (36), (3), (37), (38) and (15) could be combined. Likewise, paragraphs (16), (40), (41), (42), (29), (43), (32), (36), (3), (37), (39) and (15) could be combined.
(1) L¹ represents O;
(2) L¹ represents NR⁸;
(3) L² represents ethylene, 1-methyl-ethylene, 2-methyl-ethylene or propylene;
(4) L² represents ethylene;
(5) L³ represents O;
(6) L³ represents NR⁹;
(7) L³ represents SO₂;
(8) W represents C₁₋₆alkyl, diC₁₋₃alkylaminoC₁₋₆alkyl, C₁₋₆alkoxyC₁₋₆alkyl, C₂₋₄alkanoyl, C₁₋₄ alkylsulfonyl, C₁₋₄alkylaminocarbonyl, haloC₁₋₃alkyl or C₃₋₆cycloalkylC₁₋₃alkyl
(9) W represents C₁₋₆alkyl;
(10) W represents methyl or ethyl;
(11) W represents aryl, wherein said aryl ring is optionally substituted with 1, 2 or 3 substituents selected from R¹⁰;
(12) W represents a 5 or 6 membered monocyclic heteroaryl ring which comprises 1, 2, 3 or 4 heteroatoms independently selected from O, N or S, wherein said heteroaryl ring is optionally substituted with 1, 2 or 3 substituents selected from R¹⁰;
(13) W represents W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
(14) X represents CH;
(15) X represents N;
(16) R¹ represents hydrogen;
(17) R² represents hydrogen, halo, methyl, acetyl or trifluoromethoxy;
(18) R² represents halo;
(19) R² represents fluoro;
(20) R² represents chloro;
(21) R² represents bromo;
(22) R³ represents hydrogen;
(23) R³ represents methyl;
(24) R⁴ represents halo or C₁₋₃alkoxy;
(25) R⁴ represents chloro;
(26) R⁴ represents methoxy;
(27) R⁴ represents ethoxy;
(28) R⁴ represents morpholinomethyl;
(29) R⁵ represents hydrogen;
(30) R⁶ represents hydrogen;
(31) R⁶ represents chloro;
(32) R⁷ represents hydrogen;
(33) R⁸ represents hydrogen, methyl, ethyl or isopropyl;
(34) R⁹ represents hydrogen;
(35) R⁹ represents methyl;
(36) L¹ represents O or NR⁸, wherein R⁸ represents hydrogen, methyl, ethyl or isopropyl;
(37) L³ represents O, NR⁹ or SO₂, wherein R⁹ represents hydrogen or methyl;
(38) W represents C₁₋₆alkyl, diC₁₋₃alkylaminoC₁₋₆alkyl, C₁₋₆alkoxyC₁₋₆alkyl, C₂₋₄alkanoyl, C₁₋₄alkylsulfonyl, C₁₋₄alkylaminocarbonyl, C₃₋₆cycloalkylC₁₋₃alkyl, aryl, wherein said aryl ring is optionally substituted with 1, 2 or 3 substituents selected from R¹⁰ or W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
(39) W represents C₁₋₆alkyl, diC₁₋₃alkylaminoC₁₋₆alkyl, C₁₋₆alkoxyC₁₋₆alkyl, C₂₋₄alkanoyl, C₁₋₄alkylsulfonyl, C₁₋₄alkylaminocarbonyl, C₃₋₆cycloalkylC₁₋₃alkyl, phenyl or W and L² are joined so as to form a 4, 5, 6 or 7 membered heterocyclic ring which comprises L³;
(40) R² represents hydrogen, halo, C₁₋₃alkyl, C₂₋₃alkanoyl or trifluoromethoxy;
(41) R³ represents hydrogen or C₁₋₃alkyl;
(42) R⁴ represents halo, C₁₋₃alkoxy, haloC₁₋₃alkyl or —(CH₂)ₚ—NR¹¹R¹², wherein p represents 1 or 2 and R¹¹ and R¹² are joined so as to form a 4, 5, 6 or 7 membered heterocyclic ring which optionally comprises, in addition to the nitrogen atom to which R¹¹ and R¹² are attached, 1 or 2 further heteroatoms independently selected from O, N or S, and wherein said heterocyclic ring is optionally substituted with 1, 2 or 3 substituents selected from R¹⁰;
(43) R⁶ represents hydrogen or halo.

Particular novel compounds of Formula (I) include, but are not limited to, the following compounds:
5-Chloro-2-(3-methoxypropoxy)-N-(2-oxo-5-(2,4,6-trichlorophenyl)-2,3-dihydro-1H-benzo[e][1,4]diazepin-3-yl)benzamide;
5-Chloro-2-((S)-2-methoxypropoxy)-N-(2-oxo-5-(2,4,6-trichlorophenyl)-2,3-dihydro-1H-benzo[e][1,4]diazepin-3-yl)benzamide;
5-Chloro-2-(2-ethoxyethoxy)-N-(2-oxo-5-(2,4,6-trichlorophenyl)-2,3-dihydro-1H-benzo[e][1,4]diazepin-3-yl)benzamide;
5-Chloro-2-(1-methoxypropan-2-yloxy)-N-(2-oxo-5-(2,4,6-trichlorophenyl)-2,3-dihydro-1H-benzo[e][1,4]diazepin-3-yl)benzamide;
5-Chloro-2-(2-(2-methoxyethoxy)ethoxy)-N-(2-oxo-5-(2,4,6-trichlorophenyl)-2,3-dihydro-1H-benzo[e][1,4]diazepin-3-yl)benzamide;
5-Chloro-2-(2-isobutoxyethoxy)-N-(2-oxo-5-(2,4,6-trichlorophenyl)-2,3-dihydro-1H-benzo[e][1,4]diazepin-3-yl)benzamide;
5-Chloro-N-(2-oxo-5-(2,4,6-trichlorophenyl)-2,3-dihydro-1H-benzo[e][1,4]diazepin-3-yl)-2-(2-(pentyloxy)ethoxy)benzamide;
5-Chloro-2-(2-(2-ethoxyethoxy)ethoxy)-N-(2-oxo-5-(2,4,6-trichlorophenyl)-2,3-dihydro-1H-benzo[e][1,4]diazepin-3-yl)benzamide;
5-Chloro-N-(2-oxo-5-(2,4,6-trichlorophenyl)-2,3-dihydro-1H-benzo[e][1,4]diazepin-3-yl)-2-((tetrahydrofuran-2-yl)methoxy)benzamide;
2-(2-Butoxyethoxy)-5-chloro-N-(2-oxo-5-(2,4,6-trichlorophenyl)-2,3-dihydro-1H-benzo[e][1,4]diazepin-3-yl)benzamide;
5-Chloro-N-(2-oxo-5-(2,4,6-trichlorophenyl)-2,3-dihydro-1H-benzo[e][1,4]diazepin-3-yl)-2-(2-phenoxyethoxy)benzamide;
5-Chloro-2-(3-(dimethylamino)propoxy)-N-(2-oxo-5-(2,4,6-trichlorophenyl)-2,3-dihydro-1H-benzo[e][1,4]diazepin-3-yl)benzamide;
5-Chloro-2-(2-(2-(dimethylamino)ethoxy)ethoxy)-N-(2-oxo-5-(2,4,6-trichlorophenyl)-2,3-dihydro-1H-benzo[e][1,4]diazepin-3-yl)benzamide;
5-Chloro-2-(2-methoxyethoxy)-N-(2-oxo-5-(2,4,6-trichlorophenyl)-2,3-dihydro-1H-benzo[e][1,4]diazepin-3-yl)benzamide;
2-(2-Methoxyethoxy)-N-(2-oxo-5-(2,4,6-trichlorophenyl)-2,3-dihydro-1H-benzo[e][1,4]diazepin-3-yl)benzamide;
5-Fluoro-2-(2-methoxyethoxy)-N-(2-oxo-5-(2,4,6-trichlorophenyl)-2,3-dihydro-1H-benzo[e][1,4]diazepin-3-yl)benzamide;
2-(2-Methoxyethoxy)-5-methyl-N-(2-oxo-5-(2,4,6-trichlorophenyl)-2,3-dihydro-1H-benzo[e][1,4]diazepin-3-yl)benzamide;
5-Acetyl-2-(2-methoxyethoxy)-N-(2-oxo-5-(2,4,6-trichlorophenyl)-2,3-dihydro-1H-benzo[e][1,4]diazepin-3-yl)benzamide;
2-(2-Methoxyethoxy)-N-(2-oxo-5-(2,4,6-trichlorophenyl)-2,3-dihydro-1H-benzo[e][1,4]diazepin-3-yl)-5-(trifluoromethoxy)benzamide;
5-Bromo-2-(2-methoxyethoxy)-N-(2-oxo-5-(2,4,6-trichlorophenyl)-2,3-dihydro-1H-benzo[e][1,4]diazepin-3-yl)benzamide;
2-(2-Acetamidoethoxy)-5-chloro-N-(2-oxo-5-(2,4,6-trichlorophenyl)-2,3-dihydro-1H-benzo[e][1,4]diazepin-3-yl)benzamide;
5-Chloro-N-(2-oxo-5-(2,4,6-trichlorophenyl)-2,3-dihydro-1H-benzo[e][1,4]diazepin-3-yl)-2-(2-propionamidoethoxy)benzamide;
5-Chloro-2-(2-(methylsulfonamido)ethoxy)-N-(2-oxo-5-(2,4,6-trichlorophenyl)-2,3-dihydro-1H-benzo[e][1,4]diazepin-3-yl)benzamide;
5-Chloro-2-(2-(3-ethylureido)ethoxy)-N-(2-oxo-5-(2,4,6-trichlorophenyl)-2,3-dihydro-1H-benzo[e][1,4]diazepin-3-yl)benzamide;
2-(3-Acetamidopropoxy)-5-chloro-N-(2-oxo-5-(2,4,6-trichlorophenyl)-2,3-dihydro-1H-benzo[e][1,4]diazepin-3-yl)benzamide;
5-Chloro-N-(2-oxo-5-(2,4,6-trichlorophenyl)-2,3-dihydro-1H-benzo[e][1,4]diazepin-3-yl)-2-(3-propionamidopropoxy)benzamide;
5-chloro-2-(3-(methylsulfonamido)propoxy)-N-(2-oxo-5-(2,4,6-trichlorophenyl)-2,3-dihydro-1H-benzo[e][1,4]diazepin-3-yl)benzamide;
5-Chloro-2-(3-(ethylsulfonamido)propoxy)-N-(2-oxo-5-(2,4,6-trichlorophenyl)-2,3-dihydro-1H-benzo[e][1,4]diazepin-3-yl)benzamide;
5-Chloro-2-(2-(methylsulfonyl)ethoxy)-N-(2-oxo-5-(2,4,6-trichlorophenyl)-2,3-dihydro-1H-benzo[e][1,4]diazepin-3-yl)benzamide;
5-Fluoro-2-(2-methoxyethoxy)-N-(2-oxo-5-(2,4,6-trichlorophenyl)-2,3-dihydro-1H-benzo[e][1,4]diazepin-3-yl)nicotinamide;
2-(2-Methoxyethoxy)-N-(2-oxo-5-(2,4,6-trichlorophenyl)-2,3-dihydro-1H-benzo[e][1,4]diazepin-3-yl)nicotinamide;
5-Chloro-2-(2-methoxyethoxy)-N-(2-oxo-5-(2,4,6-trichlorophenyl)-2,3-dihydro-1H-benzo[e][1,4]diazepin-3-yl)nicotinamide;
5-Bromo-2-(2-methoxyethoxy)-N-(2-oxo-5-(2,4,6-trichlorophenyl)-2,3-dihydro-1H-benzo[e][1,4]diazepin-3-yl)nicotinamide;
2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide;
5-Fluoro-2(S)-2-methoxypropoxy)-N-(2-oxo-5-(2,4,6-trichlorophenyl)-2,3-dihydro-1H-benzo[e][1,4]diazepin-3-yl)nicotinamide;
5-Fluoro-2-(2-isopropoxyethoxy)-N-(2-oxo-5-(2,4,6-trichlorophenyl)-2,3-dihydro-1H-benzo[e][1,4]diazepin-3-yl)nicotinamide;
5-Fluoro-2-(2-(2-methoxyethoxy)ethoxy)-N-(2-oxo-5-(2,4,6-trichlorophenyl)-2,3-dihydro-1H-benzo[e][1,4]diazepin-3-yl)nicotinamide;
5-Fluoro-2-(3-methoxypropoxy)-N-(2-oxo-5-(2,4,6-trichlorophenyl)-2,3-dihydro-1H-benzo[e][1,4]diazepin-3-yl)nicotinamide;
2-(3-Ethoxypropoxy)-5-fluoro-N-(2-oxo-5-(2,4,6-trichlorophenyl)-2,3-dihydro-1H-benzo[e][1,4]diazepin-3-yl)nicotinamide;
2-(2-Methoxyethoxy)-4-methyl-N-(2-oxo-5-(2,4,6-trichlorophenyl)-2,3-dihydro-1H-benzo[e][1,4]diazepin-3-yl)nicotinamide;
5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide;
5-Fluoro-2-(2-methoxyethylamino)-N-(2-oxo-5-(2,4,6-trichlorophenyl)-2,3-dihydro-1H-benzo[e][1,4]diazepin-3-yl)nicotinamide;

2-(Ethyl(2-methoxyethyl)amino)-5-fluoro-N-(2-oxo-5-(2,4,6-trichlorophenyl)-2,3-dihydro-1H-benzo[e][1,4]diazepin-3-yl)nicotinamide;
5-Fluoro-2-(isopropyl(2-methoxyethyl)amino)-N-(2-oxo-5-(2,4,6-trichlorophenyl)-2,3-dihydro-1H-benzo[e][1,4]diazepin-3-yl)nicotinamide;
2-((2-Ethoxyethyl)(ethyl)amino)-5-fluoro-N-(2-oxo-5-(2,4,6-trichlorophenyl)-2,3-dihydro-1H-benzo[e][1,4]diazepin-3-yl)nicotinamide;
2-((2-(Cyclopropylmethoxy)ethyl)(methyl)amino)-5-fluoro-N-(2-oxo-5-(2,4,6-trichlorophenyl)-2,3-dihydro-1H-benzo[e][1,4]diazepin-3-yl)nicotinamide;
5-Chloro-N-(5-(2,6-dichlorophenyl)-2-oxo-2,3-dihydro-1H-benzo[e][1,4]diazepin-3-yl)-2-(2-methoxyethoxy)benzamide;
N-(5-(2,6-Dichlorophenyl)-2-oxo-2,3-dihydro-1H-benzo[e][1,4]diazepin-3-yl)-5-fluoro-2-(2-methoxyethoxy)benzamide;
5-Chloro-N-(5-(2,4-dichloro-6-(morpholinomethyl)phenyl)-2-oxo-2,3-dihydro-1H-benzo[e][1,4]diazepin-3-yl)-2-(2-methoxyethoxy)benzamide;
N-(5-(2,4-Dichloro-6-(morpholinomethyl)phenyl)-2-oxo-2,3-dihydro-1H-benzo[e][1,4]diazepin-3-yl)-5-fluoro-2-(2-methoxyethoxy)benzamide;
5-Chloro-N-(5-(2,4-dichloro-6-(morpholinomethyl)phenyl)-2-oxo-2,3-dihydro-1H-benzo[e][1,4]diazepin-3-yl)-2-(2-methoxyethoxy)nicotinamide;
N-(5-(2,4-Dichloro-6-(morpholinomethyl)phenyl)-2-oxo-2,3-dihydro-1H-benzo[e][1,4]diazepin-3-yl)-5-fluoro-2-(2-methoxyethoxy)nicotinamide;
N-(5-(2,4-Dichloro-6-methoxyphenyl)-2-oxo-2,3-dihydro-1H-benzo[e][1,4]diazepin-3-yl)-5-fluoro-2-(2-methoxyethoxy)benzamide;
N-(5-(2,4-Dichloro-6-methoxyphenyl)-2-oxo-2,3-dihydro-1H-benzo[e][1,4]diazepin-3-yl)-5-fluoro-2-(2-methoxyethoxy)nicotinamide;
N-(5-(2,4-Dichloro-6-methoxyphenyl)-2-oxo-2,3-dihydro-1H-benzo[e][1,4]diazepin-3-yl)-2-(2-ethoxyethoxy)-5-fluoronicotinamide;
N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide;
N-(5-(2,4-Dichloro-6-ethoxyphenyl)-2-oxo-2,3-dihydro-1H-benzo[e][1,4]diazepin-3-yl)-5-fluoro-2-(2-methoxyethoxy)nicotinamide;
N-(5-(2,4-Dichloro-6-ethoxyphenyl)-2-oxo-2,3-dihydro-1H-benzo[e][1,4]diazepin-3-yl)-2-(2-ethoxyethoxy)-5-fluoronicotinamide;
(S)-5-Chloro-2-(2-methoxyethoxy)-N-(2-oxo-5-(2,4,6-trichlorophenyl)-2,3-dihydro-1H-benzo[e][1,4]diazepin-3-yl)benzamide;
(R)-5-Chloro-2-(2-methoxyethoxy)-N-(2-oxo-5-(2,4,6-trichlorophenyl)-2,3-dihydro-1H-benzo[e][1,4]diazepin-3-yl)benzamide;
(S)-5-Fluoro-2-(2-methoxyethoxy)-N-(2-oxo-5-(2,4,6-trichlorophenyl)-2,3-dihydro-1H-benzo[e][1,4]diazepin-3-yl)benzamide;
(R)-5-Fluoro-2-(2-methoxyethoxy)-N-(2-oxo-5-(2,4,6-trichlorophenyl)-2,3-dihydro-1H-benzo[e][1,4]diazepin-3-yl)benzamide;
(S)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide;
(R)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide;
(S)-2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide;
(R)-2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide;
(S)-5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide;
(R)-5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide;
(S)-N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide;
(R)-N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide;
and pharmaceutically acceptable salts thereof

A particular novel compound of Formula (I) is (5-Fluoro-2-(2-methoxyethoxy)-N-(2-oxo-5-(2,4,6-trichlorophenyl)-2,3-dihydro-1H-benzo[e][1,4]diazepin-3-yl)nicotinamide and pharmaceutically acceptable salts thereof

A particular novel compound of Formula (I) is (S)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide and pharmaceutically acceptable salts thereof

A suitable pharmaceutically acceptable salt of a compound of Formula (I) is, for example, where the compound is sufficiently basic, an acid addition salt such as a hydrochloride, hydrobromide, phosphate, acetate, fumarate, maleate, tartrate, citrate, oxalate, methanesulfonate or p-toluenesulfonate salt. There may be more than one anion depending on the number of charged functions and the valency of the anions. Other is pharmaceutically acceptable salts, as well as pro-drugs such as pharmaceutically acceptable esters and pharmaceutically acceptable amides may be prepared using conventional methods.

For example, the compounds of the invention may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable amide derivatives that may be formed at an amino group in a compound of Formula (I).

Accordingly, the present invention includes those compounds of Formula (I) as defined hereinbefore when made available by organic synthesis and when made available within the human or animal body by way of cleavage of a pro-drug thereof Accordingly, the present invention includes those compounds of Formula (I) that are produced by organic synthetic means and also such compounds that are produced in the human or animal body by way of metabolism of a precursor compound, that is a compound of Formula (I) may be a synthetically-produced compound or a metabolically-produced compound.

A suitable pharmaceutically acceptable pro-drug of a compound of Formula (I) is one that is based on reasonable medical judgement as being suitable for administration to the human or animal body without undesirable pharmacological activities and without undue toxicity.

Various forms of pro-drug have been described, for example in the following documents:
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of Formula (I) that possesses an amino group is, for example, an in vivo cleavable amide derivative thereof. Suitable pharmaceutically acceptable amides from an amino group include, for example an amide formed with C₂₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl)piperazin-1-ylmethyl.

The in vivo effects of a compound of Formula (I) may be exerted in part by one or more metabolites that are formed within the human or animal body after administration of a compound of Formula (I). As stated hereinbefore, the in vivo effects of a compound of Formula (I) may also be exerted by way of metabolism of a precursor compound (a pro-drug).

### Preparation of Compounds of Formula (I)

Certain processes for the synthesis of compounds of Formula (I) are provided as a s further feature of the invention. Thus, according to a further aspect of the invention there is provided a process for the preparation of a compound of Formula (I), or a pharmaceutically acceptable salt thereof, which comprises a process (a), (b) or (c) wherein, unless otherwise defined, the variables are as defined hereinbefore for compounds of Formula (I):
(a) reaction of a compound of Formula (II), or a salt thereof, with a compound of Formula (III) in the presence of a suitable coupling agent and a suitable base:

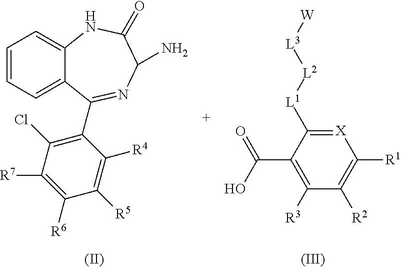

(b) reaction of a compound of Formula (IV) with a compound of Formula (III) in the presence of a suitable coupling agent and a suitable base, wherein P¹ represents a suitable protecting group:

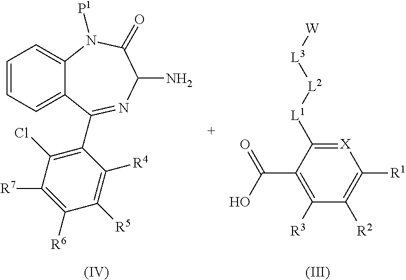

(c) when L¹ represents NR⁸, reaction of a compound of Formula (V) with an amine of Formula (VI) wherein Y represents a suitable leaving group:

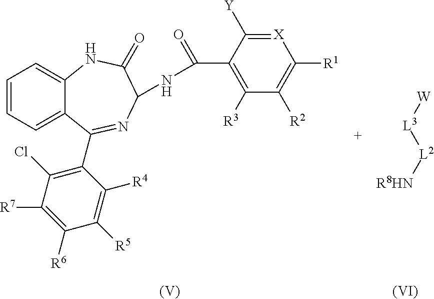

and thereafter, if necessary:
(i) converting a compound of Formula (I) into another compound of Formula (I);
(ii) removing any protecting groups;
(iii) separating a racemic mixture into separate enantiomers; and/or
(iv) preparing a pharmaceutically acceptable salt thereof.

Intermediate compounds may be prepared by suitable procedures, for example as known in the art by skilled persons. For example, compounds of Formulae (II) and (IV) may be prepared as follows:
(d) Compounds of Formula (II) wherein R⁴ represents chloro may be prepared by reacting a compound of Formula (VII) with a compound of Formula (VIII) in the presence of silver and a suitable catalyst wherein P¹ and P² represent suitable protecting groups:

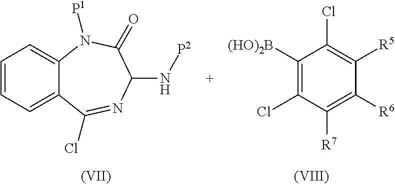

and thereafter removing the protecting groups.
(e) Compounds of Formula (IV) wherein R⁴ represents chloro may be prepared by reacting a compound of Formula (VII) with a compound of Formula (VIII) in the presence of silver

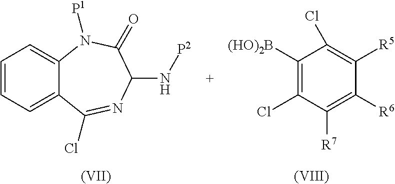

and thereafter removing the P² protecting group.

Specific reaction conditions for processes (a), (b), (c), (d) and (e) above are as follows:
Process (a)—a compound of Formula (II) may be reacted with a compound of Formula (III) in the presence of a suitable coupling agent, for example HBTU, optionally in the presence of a suitable base, for example TEA, a suitable solvent, for example DMF, and at a suitable temperature, for example room temperature.
Process (b) - a compound of Formula (IV) wherein P¹ represents a suitable protecting group, for example PMB, may be reacted with a compound of Formula (III) in the presence of a suitable coupling agent, for example HBTU, optionally in the presence of a suitable base, for example TEA, a suitable solvent, for example DMF, and at a suitable temperature, for example room temperature.
Process (c)—a compound of Formula (V) wherein Y represents a suitable leaving group, is for example fluoro, chloro, bromo, iodo, mesylate or tosylate, may be reacted with an amine of Formula (VI) in a suitable solvent, for example a 4:1 mixture of 1,4-dioxane in water, by heating to a suitable temperature, for example 100 to 200° C., more suitably about 160° C., using a suitable heat source, for example microwave radiation.
Processes (d) and (e)—a compound of Formula (VII) wherein P and P² represent suitable protecting groups, for example PMB and tert-butyloxycarbonyl respectively, may be reacted with a compound of Formula (VIII) in a suitable solvent, for example THF, in the presence of silver, for example Ag₂CO₃, a suitable catalyst, for example tetrakis(triphenylphosphine)palladium(0), and optionally in the presence of a suitable base, for example K₂CO₃, by heating to a suitable temperature, for example reflux temperature.

A process for the preparation of compounds of Formula (I) may comprise converting a compound of Formula (I) into another compound of Formula (I) using standard chemical reactions well-known to those skilled in the art to produce another compound of the invention. Chemical conversions of this type are well known to those skilled in the art and may include functional group interconversions such as hydrolysis, hydrogenation, hydrogenolysis, oxidation or reduction, and/or further functionalisation by standard reactions such as amide or metal-catalysed coupling, or nucleophilic displacement reactions. Examples of such conversions are described, for instance, in Comprehensive Organic Chemistry, Volume 2, p 3, D. Barton and D. Ollis Eds, Pergamon, 1979, Comprehensive Functional Group Transformations, A. R. Katritzky, O. Meth-Cohn, and C. W. Rees Eds., Pergamon, 1995, and by various authors in Houben-Weyl, Methods of Organic Chemistry, Verlag Chemie, various years, and references therein.

It will be appreciated by a person skilled in the art that it may be necessary/desirable to protect any sensitive groups in the compounds in some of the is processes/ reactions mentioned herein. The instances where protection is necessary or desirable, and suitable methods for providing such protection are known to those skilled in the art. Conventional protecting groups may be used in accordance with standard practice (for illustration see P. G. M. Wuts and T. W. Green, Protective Groups in Organic Synthesis, 4th Edition, John Wiley and Sons, 2002). Thus, if reactants include groups such as amino, carboxy or hydroxy it may be desirable to protect the group in some of the reactions mentioned herein.

Any protecting groups utilised in the processes described herein may in general be chosen from any of the groups described in the literature or known to the skilled chemist as appropriate for the protection of the group in question and may be introduced by conventional methods. Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule. The protecting groups may be removed at any convenient stage in the synthesis using conventional techniques well known in the chemical art.

For example, in process (b) described above, a suitable protecting group P¹ would be p-methoxybenzyl. Once the reaction described in process (b) is complete, the p-methoxybenzyl can be removed by treating with aluminium trichloride as described in General Method F.

A process for the manufacture of compounds of Formula (I) in the form of a single enantiomer may comprise separating a racemic compound of the invention into separate enantiomers.

Examples of suitable methods for separating the enantiomers of a racemic compound are well known to those skilled in the art and include chromatography using a suitable chiral stationary phase; or conversion of a racemic mixture into diastereomeric derivatives, separation of the mixture of diastereomeric derivatives into two single diastereomers, and regeneration of a separate single enantiomer from each separate single diastereomer; or selective chemical reaction of one of the enantiomers of a racemic compound (kinetic resolution) using a diastereoselective reaction catalysed by a microbiological agent or an enzyme.

Alternatively, compounds of the invention in the form of a single enantiomer may is be prepared by using chiral starting materials to carry out one of the processes described above.

Biological Assays The ability of compounds to inhibit HCV Polymerase activity and replication of an HCV replicon was assessed using the assays described below.

### (a) HCV Polymerase Enzyme Assay

Compounds were tested for inhibition of HCV polymerase using a radiometric [³³P]-UTP incorporation assay and a biotinylated U₁₃:PolyA primer:template RNA substrate.

Recombinant HCV polymerase (BK strain) was expressed and purified from E. coli with a 21 amino acid C-terminal deletion and a His₆-tag. The general assay buffer consisted of 20 mM Tris (pH 7.5), 25 mM KCl, 5 mM MgCl₂, 3 mM DTT, 0.5 mg/ml BSA, 0.01% Tween20. The standard reaction, in 96 well plates, contained 10 μl of diluted compound, 50 μl of substrate and 40 μl of enzyme. Compounds, supplied as 10 mM stocks in DMSO, were diluted initially in neat DMSO, and subsequently buffer was added to give a DMSO concentration of 30%; 10 μl of this was added to the assay plate to give a final concentration in the 100 μl assay of 3% DMSO. The biotinylated U₁₃:PolyA RNA substrate was pre-annealed with 40 μM 5′-biotinylated U₁₃ (Dharmacon) and 213 μg/ml PolyA (Amersham Biosciences) in water incubated at 70° C. for 5 minutes before being cooled on ice. Substrate (50 μl) was added in buffer to give a final concentration in the 100 μl assay of 125 nM biotinylated U₁₃:0.63 μg/ml PolyA and 200 nM UTP with 0.4 μCi [³³P]-UTP per well (Perkin Elmer). The reaction was initiated by the addition of 40 μl of enzyme in buffer to give 100 nM final concentration. The reaction was incubated at 25° C. for 100 minutes and then stopped by addition of 100 μl of 100 mM EDTA. The samples were then transferred to 96 well Streptavidin-coated FlashPlates (Perkin Elmer) and incubated at room temperature for ˜1 hour to allow binding to occur. The plates were then washed three times with phosphate-buffered saline containing 0.05% Tween20 in an automated plate washer to remove unincorporated [³³P]-UTP, and then counted in a

### Packard TopCount Scintillation Counter.

Each plate included a set of positive controls (no compound, maximum signal) and negative controls (no enzyme, minimum signal) and in each run at least one reference is compound was included to validate the assay. The IC₅₀, concentration required to inhibit the enzyme activity by 50%, was calculated using an 8-point IC₅₀ curve and fitted using the program XLfit (IDBS).

### (b) HCV Replicon Assay

### Cells Used:

HCV replicon cells Huh 9B (ReBlikon), containing the firefly luciferase—ubiquitin—neomycin phosphotransferase fusion protein and EMCV-IRES driven HCV polyprotein with cell culture adaptive mutations.

### Cell Culture Conditions:

Cells were cultured at 37° C. in a 5% CO₂ environment and split twice a week on seeding at 2×10⁶ cells/flask on day 1 and 1×10⁶ 3 days later. G418 at 0.5 mg/ml was added to the culture medium but not the assay medium. The culture medium consisted of DMEM with 4500 g/l glucose and glutamax (Gibco 61965-026) supplemented with 1×non-essential amino acids (Invitrogen 11140-035), penicillin (100 IU/ml)/streptomycin (100 μg/ml) (Invitrogen 15140-122), FCS (10%, 50 ml) and 1 mg/ml G418 (Invitrogen 10131-027) & 10% Australian foetal calf serum (Invitrogen 10099-141).

### Assay Procedure:

A flask of cells was trypsinised and a cell count carried out. Cells were diluted to 100,000 cells/ml and 100 μl of this used to seed one opaque white 96-well plate (for the replicon assay) and one flat-bottomed clear plate (for the tox assay) for every five compounds to be tested for IC₅₀. Wells G12 and H12 were left empty in the clear plate as the blank. Plates were then incubated at 37° C. in a 5% CO₂ environment for 24 h.

On the following day compound dilutions are made up in medium at twice their desired final concentration in a clear round bottomed plate. All dilutions have a final DMSO concentration of 1%.

Once the dilution plate had been made up, controls and compounds were transferred to the assay plates (containing the cells) at 100 μl/well in duplicate wells. Exception: no compound was added to wells A1 and A2 of either plate and 100 μl of 1% DMSO was added to these instead. Plates were then incubated at 37° C. with 5% CO₂ for 72 h.

At the end of the incubation time, the cells in the white plate were harvested by washing in PBS (100 μL per well) and gently tapping dry before addition of 204 per well of lysis buffer (25 mM tris-phosphate, 8 mM MgCl₂, 1 mM DTT, 1% Triton X-100, 15% glycerol. pH to 7.8 using KH₂PO₄ prior to triton and glycerol addition. Substrate was prepared: 23.5 mM beetle luciferin (Promega E1603), 26 mM ATP (Sigma O-2060) in 100 nM Tris buffer pH 7.8 aliquoted and stored at −80° C. was thawed and diluted 1:50 in luciferase assay buffer (20 mM Tricine (Sigma T-0377), 1.07 mM magnesium carbonate hydroxide (Sigma M-5671), 0.1 mM EDTA (Sigma E-5134), 2.67 mM MgSO₄(BDH 101514Y), 33.3 mM dithiothreitol (Sigma 150460) pH 7.8).

The M injector of the microplate luminometer (Lmax, Molecular Devices) was primed with 5×300 μl injections of the diluted substrate. After 5-60 min incubation in lysis buffer at room temperature, a plate was inserted into the luminometer and 100 μl luciferase assay reagent was added by the injector on the luminometer. The signal was measured using a 1 second delay followed by a 4 second measurement programme. The IC₅₀, the concentration of the drug required for reducing the replicon level by 50% in relation to the untreated cell control value, can be calculated from the plot of the percentage reduction of the luciferase activity vs. drug concentration.

The clear plate was stained with 100 μl 0.5% methylene blue in 50% ethanol at room temperature for lh, followed by solvation of the absorbed methylene blue in 100 μl per well of 1% lauroylsarcosine. Absorbance of the plate was measured on a microplate spectrophotometer (Molecular Devices) and the absorbance for each concentration of compound expressed as a proportion of the relative DMSO control. The TD₅₀, the concentration of drug required to reduce the total cell area by 50% relative to the DMSO controls, can be calculated by plotting the absorbance at 620 nm minus background against drug concentration.

### Results

When tested in assays (a) and (b) described above, all of the compounds of the Examples gave IC₅₀ values for HCV polymerase inhibitory activity and/or reduction of replicon levels of less than 10 μM (micromolar), indicating that the compounds of the invention are expected to possess useful therapeutic properties. The IC₅₀ and TD₅₀ values so obtained are shown in the following Table:

| | HCV 1b | | |
|---|---|---|---|
| Example | Polymerase Assay | Replicon Assay | Replicon Assay |
| Number | Value IC₅₀ (μM) | Value IC₅₀ (μM) | Value TD₅₀ (μM) |
| 1 | 0.354 | 0.21 | >25 |
| 2 | 0.1673 | 0.3815 | >25 |
| 3 | 0.1115 | 0.0829 | 16.177 |
| 4 | 0.2605 | 0.3035 | >25 |
| 5 | 0.209 | 0.4902 | 15.913 |
| 6 | 0.715 | 0.849 | 10.604 |
| 7 | 0.6615 | 1.4453 | 11.806 |
| 8 | 1.068 | 1.3398 | 11.724 |
| 9 | 0.296 | 0.6104 | >25 |
| 10 | 0.757 | 0.338 | >25 |
| 11 | 0.864 | 1.1453 | >25 |
| 12 | 3.1333 | 5.5098 | 4.253 |
| 13 | 1.7035 | 3.1713 | 6.754 |
| 14 | 0.1536 | 0.0833 | 19.834 |
| 15 | 0.299 | 0.1098 | 25.845 |
| 16 | 0.119 | 0.0387 | 22.094 |
| 17 | 0.2345 | 0.0788 | >25 |
| 18 | 0.385 | 0.8763 | 15.802 |
| 19 | 2.154 | 0.6452 | >25 |
| 20 | 0.535 | 0.1954 | >25 |
| 21 | 0.0987 | 0.824 | 13.188 |
| 22 | 0.1975 | 0.9526 | 12.304 |
| 23 | 0.952 | 2.9052 | 12.744 |
| 24 | 0.2805 | 1.276 | 11.815 |
| 25 | 0.4585 | 0.5749 | 12.774 |
| 26 | 0.647 | 1.6228 | 12.354 |
| 27 | 0.824 | 1.9113 | 13.62 |
| 28 | 0.8495 | 2.1318 | 8.004 |
| 29 | 0.747 | 2.7726 | 15.249 |
| 30 | 0.1163 | 0.0464 | >25 |
| 31 | 0.2475 | 0.2339 | >25 |
| 32 | 0.1784 | 0.1357 | >25 |
| 33 | 0.265 | 0.487 | >25 |
| 34 | 0.091 | 0.0141 | 11.884 |
| 35 | 0.0897 | 0.1843 | 24.614 |
| 36 | 0.2435 | 0.3075 | 13.55 |
| 37 | 0.1015 | 0.2627 | 23.09 |
| 38 | 0.079 | 0.0157 | 22.269 |
| 39 | 0.095 | 0.0663 | 11.83 |
| 40 | 2.9645 | 0.8556 | 24.518 |
| 41 | 0.1294 | 0.0247 | 24.146 |
| 42 | 0.9685 | 2.6406 | 17.784 |
| 43 | 0.2045 | 0.7872 | 23.97 |
| 44 | 1.3275 | 2.648 | 23.394 |
| 45 | 0.1758 | 0.7516 | 18.864 |
| 46 | 0.1677 | 0.151 | 15.961 |
| 47 | 0.1494 | 0.5833 | >25 |
| 48 | 0.1552 | 0.7034 | >25 |
| 49 | 0.829 | 0.1671 | 11.02 |
| 50 | 0.6657 | 0.2077 | 11.74 |
| 51 | 1.2285 | 0.2811 | >25 |
| 52 | 0.6053 | 0.1294 | 24.829 |
| 53 | 0.1545 | 0.0595 | 24.827 |
| 54 | 0.108 | 0.0184 | >25 |
| 55 | 0.1165 | 0.027 | 18.821 |
| 56 | 0.1535 | 0.0389 | 11.349 |
| 57 | 0.072 | 0.0203 | 22.698 |
| 58 | 0.069 | 0.0099 | 11.875 |
| 59 | 0.0876 | 0.0453 | >25 |
| 60 | 27.695 | 0.7978 | >25 |
| 61 | 0.0444 | 0.0167 | 17.347 |
| 62 | 16.778 | 0.5942 | 24.765 |
| 63 | 0.0505 | 0.0097 | 17.386 |
| 64 | 2.0669 | 0.2408 | 23.452 |
| 65 | 0.0323 | 0.0048 | 10.854 |
| 66 | 2.2232 | 0.1628 | 16.686 |
| 67 | 0.0363 | 0.0112 | 13.07 |
| 68 | 5.169 | 0.3597 | 16.618 |
| 69 | 0.0792 | 0.0313 | 17.731 |
| 70 | 5.356 | 0.737 | 24.592 |

### Pharmaceutical Compositions and Methods of Treatment Comprising Compounds of Formula (I)

The compounds of Formula (I) and pharmaceutically acceptable salts thereof may be used on their own but will generally be administered in the form of a pharmaceutical composition in which the Formula (I) compound/salt (active ingredient) is in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of Formula (I) or a pharmaceutically acceptable salt thereof as hereinbefore defined, in association with a pharmaceutically acceptable adjuvant, diluent or carrier.

The invention further provides a process for the preparation of a pharmaceutical composition of the invention which comprises mixing a compound of Formula (I) or a pharmaceutically acceptable salt thereof as hereinbefore defined with a pharmaceutically acceptable adjuvant, diluent or carrier.

The compounds of the invention may be administered in a variety of dosage forms. Thus, they can be administered orally, for example as tablets, troches, lozenges, aqueous or oily suspensions, dispersible powders or granules. The compounds of the invention may also be administered parenterally, whether subcutaneously, intravenously, intramuscularly, intrasternally, transdermally or by infusion techniques. The compounds may also be administered as suppositories.

The compounds of the invention are typically formulated for administration with a pharmaceutically acceptable carrier or diluent. For example, solid oral forms may contain, together with the active compound, diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, arabic gums, gelatin, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulfates; and, in general, non toxic and pharmacologically inactive substances used in pharmaceutical formulations. Such pharmaceutical preparations may be manufactured in known manner, for example, by means of mixing, granulating, tableting, sugar coating, or film coating processes.

Liquid dispersions for oral administration may be syrups, emulsions and suspensions. The syrups may contain as carriers, for example, saccharose or saccharose with glycerine and/or mannitol and/or sorbitol.

Suspensions and emulsions may contain as carrier, for example a natural gum, agar, sodium alginate, pectin, methylcellulose, carboxymethylcellulose, or polyvinyl alcohol.

The suspension or solutions for intramuscular injections may contain, together with the active compound, a pharmaceutically acceptable carrier, e.g. sterile water, olive oil, ethyl oleate, glycols, e.g. propylene glycol, and if desired, a suitable amount of lidocaine hydrochloride.

Solutions for injection or infusion may contain as carrier, for example, sterile water or preferably they may be in the form of sterile, aqueous, isotonic saline solutions.

The compound of Formula (I) will normally be administered to a warm-blooded animal at a unit dose within the range 5-5000 mg/m² body area of the animal, i.e. approximately 0.1-100 mg/kg, and this normally provides a therapeutically-effective dose. A unit dose form such as a tablet or capsule will usually contain, for example 1-250 mg of active ingredient. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The compounds of Formula (I) and their pharmaceutically acceptable salts have activity as pharmaceuticals, in particular as antiviral agents and especially as agents for the treatment of Flaviviridae infections. More particularly, the compounds of Formula (I) and their pharmaceutically acceptable salts may be used in the treatment of hepatitis C virus infection.

Thus, the present invention provides a compound of Formula (I), or a pharmaceutically-acceptable salt thereof, as defined hereinbefore for use in therapy.

In a further aspect, the present invention provides a compound of Formula (I), or a pharmaceutically acceptable salt thereof, as defined hereinbefore for use in the treatment or prophylaxis of hepatitis C virus infection.

In a further aspect, the present invention provides the use of a compound of Formula (I), or a pharmaceutically acceptable salt thereof, as defined hereinbefore in the manufacture of a medicament for use in the treatment or prophylaxis of hepatitis C virus.

In a further aspect, the present invention provides a method of treating, or reducing the risk of, hepatitis C virus infection in a warm-blooded animal, such as man, in need of such treatment which comprises administering to said animal an effective amount of a compound of Formula (I), or a pharmaceutically acceptable salt thereof, as defined hereinbefore.

In the context of the present specification, the term “therapy” also includes “prophylaxis” unless there are specific indications to the contrary. The terms “therapeutic” and “therapeutically” should be construed accordingly.

Prophylaxis is expected to be particularly relevant to the treatment of persons who have suffered a previous episode of, or are otherwise considered to be at increased risk of, HCV infection. Persons at risk of developing a particular disease or condition generally include those having a family history of the disease or condition, or those who have been identified by genetic testing or screening to be particularly susceptible to developing the disease or condition.

The compounds of the invention may also be administered in conjunction with other compounds used for the treatment of viral infections. Thus, the invention further relates to combination therapies for the treatment of a viral infection, particularly infection by hepatitis C virus, wherein a compound of Formula (I), or a pharmaceutically acceptable salt thereof, as defined hereinbefore or a pharmaceutical composition or formulation comprising a compound of Formula (I), is administered concurrently or sequentially or as a combined preparation with another therapeutic agent or agents.

In particular the compounds of the invention may be administered in conjunction with one or more further active ingredients that are selected from:
(a) a HCV protease inhibitor, for example IDX-320, MK-5172, IDX-320, BMS-650032, ACH-2684, ACH-1625, BI-1335, TMC435350, MK7009, ITMN-191, BILN-2061, VX-950, BILN-2065, BMS-605339, VX-500 and SCH 503034;
(b) a HCV polymerase inhibitor, for example ABT-333, ABT-072, IDX-184, ANA598, VX-222, PSI-938, PSI-7977, R-7128, MK-0608, VCH759, PF-868554, GS9190, NM283, valopicitabine, PSI-6130, XTL-2125, NM-107, R7128 (R4048), GSK625433, R803, R-1626, BILB-1941, HCV-796, JTK-109 and JTK-003, benzimidazole derivatives, benzo-1,2,4-thiadiazine derivatives, phenylalanine derivatives,;
(c) a HCV helicase inhibitor;
(d) an immunomodulatory agent, for example α-, β-, and γ-interferons such as rIFN-α 2b, rIFN-α 2ba, consensus IFN-α (infergen), feron, reaferon, intermax α, rIFN-β, infergen+actimmune, IFN-omega with DUROS, albuferon, locteron, Rebif, Oral IFN-α, IFN-α 2b XL, AVI-005, pegylated-infergen, pegylated derivatized interferon-a compounds such as pegylated rIFN-α 2b, pegylated rIFN-α 2a, pegylated IFN-β, compounds that stimulate the synthesis of interferon in cells, interleukins, Toll like receptor (TLR) agonists, compounds that enhance the development of type 1 helper T cell response and thymosin;
(e) a HCV NS5a inhibitor such as A-831/AZD2836 (i.e. [6-(3,4-Dimethoxy-phenyl)-quinazolin-4-yl]-(4-[1,2,4]triazol-1-yl-phenyl)-amine, or a pharmaceutically acceptable salt thereof) and A-689/AZD7295 (i.e. 5′-(cyclopropanecarbonyl-amino)-2′-trifluoromethoxy-biphenyl-4-carboxylic acid {4-[4-(propane-1-sulfonyl)-piperazin-1-ylmethyl]-phenyl}-amide, or a pharmaceutically acceptable salt thereof), PPI-461 or BMS-790052;
(f) other antiviral agents, for example ribavirin, ribavirin analogs such as rebetol, copegus and viramidine (taribavirin), amantadine, and telbivudine, inhibitors of internal ribosome entry, alpha-glucosidase 1 inhibitors such as MX-3253 (celgosivir) and UT-231B, hepatoprotectants such as IDN-6556, ME-3738, LB-84451 and MitoQ, broad-spectrum viral inhibitors, such as IMPDH inhibitors (e.g., mycophenolic acid and derivatives thereof, and VX-497, VX-148, and/or VX-944); and
(g) other drugs for treating HCV such as zadaxin, nitazoxanide, BIVN-401 (virostat), PYN-17 (altirex), KPE02003002, actilon (CPG-10101), KRN-7000, civacir, GI-5005, ANA-975, XTL-6865, ANA-971, NOV-205, tarvacin, EHC-18, NIM811, DEBIO-025, VGX-410C, EMZ-702, AVI 4065, Bavituximab, and Oglufanide.

For example, the compounds of the invention may be administered in conjunction with one or more further active ingredients that are selected from:
(a) a HCV protease inhibitor, for example BI-1335, TMC435350, MK70009, ITMN-191, BILN-2061, VX-950, BILN-2065, BMS-605339, VX-500 and SCH 503034;
(b) a HCV polymerase inhibitor, for example R-7128, MK-0608, VCH759, PF-868554, GS9190, NM283, valopicitabine, PSI-6130, XTL-2125, NM-107, R7128 (R4048), GSK625433, R803, R-1626, BILB-1941, HCV-796, JTK-109 and JTK-003, benzimidazole derivatives, benzo-1,2,4-thiadiazine derivatives, phenylalanine derivatives;
(c) a HCV helicase inhibitor;
(d) an immunomodulatory agent, for example α-, β-, and γ-interferons such as rIFN-α 2b, rIFN-α 2ba, consensus IFN-α (infergen), feron, reaferon, intermax α, rIFN-β, infergen+actimmune, IFN-omega with DUROS, albuferon, locteron, Rebif, Oral IFN-α, IFN-α 2b XL, AVI-005, pegylated-infergen, pegylated derivatized interferon-a compounds such as pegylated rIFN-α 2b, pegylated rIFN-α 2a, pegylated IFN-β, compounds that stimulate the synthesis of interferon in cells, interleukins, Toll like receptor (TLR) agonists, compounds that enhance the development of type 1 helper T cell response and thymosin;
(e) a HCV NS5a inhibitor such as A-831/AZD2836 and A-689/AZD7295 or BMS-790052;
(f) other antiviral agents, for example ribavirin, ribavirin analogs such as rebetol, copegus and viramidine (taribavirin), amantadine, and telbivudine, inhibitors of internal ribosome entry, alpha-glucosidase 1 inhibitors such as MX-3253 (celgosivir) and UT-231B, hepatoprotectants such as IDN-6556, ME-3738, LB-84451 and MitoQ, broad-spectrum viral inhibitors, such as IMPDH inhibitors (e.g., mycophenolic acid and derivatives thereof, and VX-497, VX-148, and/or VX-944); and
(g) other drugs for treating HCV such as zadaxin, nitazoxanide, BIVN-401 (virostat), PYN-17 (altirex), KPE02003002, actilon (CPG-10101), KRN-7000, civacir, GI-5005, ANA-975, XTL-6865, ANA-971, NOV-205, tarvacin, EHC-18, NIM811, DEBIO-025, VGX-410C, EMZ-702, AVI 4065, Bavituximab, and Oglufanide.

In one embodiment of the invention, there is provided a therapeutic combination which comprises a compound of Formula (I), or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor.

In another embodiment of the invention, there is provided a therapeutic is combination which comprises a compound of Formula (I), or a pharmaceutically acceptable salt thereof, and an interferon, ribavirin and VX950.

In one embodiment of the invention, there is provided a combination product which comprises a compound of Formula (I), or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor.

In another embodiment of the invention, there is provided a combination product which comprises a compound of Formula (I), or a pharmaceutically acceptable salt thereof, and an interferon, ribavirin and VX950. The term “therapeutic combination” as referred to in this description in intended to mean any combination of the specified pharmaceutical agents that produces a therapeutic effect upon administration.

The term “combination product” as referred to in this description in intended to mean any product that comprises a compound of Formula (I), or a pharmaceutically acceptable salt thereof, and another specified pharmaceutical agent or agents and includes, but is not limited to, an individual pharmaceutical preparation comprising both a compound of Formula (I) and another specified pharmaceutical agent or agents (i.e. a combined preparation), a kit of parts comprising pharmaceutical preparations of a compound of Formula (I) and another specified pharmaceutical agent or agents as individual or separate preparations, storage means for pharmaceutical preparations of a compound of Formula (I) and another specified pharmaceutical agent or agents as either individual or separate preparations and/or means for dispensing pharmaceutical preparations of a compound of Formula (I) and another specified pharmaceutical agent or agents as either individual or separate preparations, wherein the term “individual pharmaceutical preparation” or “individual preparations” is intended to mean a single pharmaceutical preparation which comprises both a compound of Formula (I) and another specified pharmaceutical agent or agents and wherein the term “separate preparations” is intended to mean two or more different pharmaceutical preparations one of which comprises a compound of Formula (I) and the others of which each comprise another specified pharmaceutical agent.

In a further aspect of the invention, there is provided a method of treating hepatitis C virus infection by administering to a patient in need thereof an effective amount of a is compound of Formula (I), or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor.

In one embodiment of the invention, there is provided a method of treating hepatitis C virus infection by administering to a patient in need thereof an effective amount of a compound of Formula (I), or a pharmaceutically acceptable salt thereof, and an interferon, ribavirin and VX950.

In another aspect of the invention, there is provided a therapeutic combination or a combination product comprising a compound of Formula (I), or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor, for use in the treatment of hepatitis C virus infection.

In one embodiment of the invention, there is provided a therapeutic combination or a combination product comprising a compound of Formula (I), or a pharmaceutically acceptable salt thereof, and an interferon, ribavirin and VX950, for use in the treatment of hepatitis C virus infection.

In another aspect, the present invention provides the use of a therapeutic combination or a combination product comprising a compound of Formula (I), or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor, in the manufacture of a medicament for the treatment of hepatitis C virus infection.

In one embodiment, the present invention provides the use of a therapeutic combination or a combination product comprising a compound of Formula (I), or a pharmaceutically acceptable salt thereof, and an interferon, ribavirin and VX950, in the manufacture of a medicament for the treatment of hepatitis C virus infection.

### EXAMPLES

The present invention will now be further explained by reference to the following illustrative examples in which, generally:
(i) temperatures are given in degrees Celsius (° C.); unless stated otherwise, operations were is carried out at room or ambient temperature, that is, at a temperature in the range of 18 to 25° C.;
(ii) Organic solutions were dried over anhydrous magnesium sulfate; evaporation of solvent was carried out using a rotary evaporator under reduced pressure (1-750 mbar) with a bar temperature up to 60° C.;
(iii) chromatography means flash chromatography on silica gel;
(iv) in general, the course of reactions was followed by analytical LC-MS, and reaction times where given are for illustration only.
(v) final products had satisfactory proton nuclear magnetic resonance (NMR) spectra and/or mass spectral data;
(vi) yields are given for illustration only and are not necessarily those which can be obtained by diligent process development; preparations were repeated if more material was required;
(vii) when given, NMR data is in the form of delta values for major diagnostic protons, given in parts per million (ppm) relative to tetramethylsilane, determined at 250 MHz, using perdeuterio dimethyl sulfoxide (d₆-DMSO) as solvent, unless otherwise stated; the following abbreviations have been used: s, singlet; d, doublet; t, triplet; q, quartet; m, multiplet; br, broad; coupling constants, J, are reported in Hz;
(viii) chemical symbols have their usual meanings; SI units and symbols are used;
(ix) LC-MS was carried out using one of five methods:

1. (QC Method 1)

Liquid Chromatograph: Agilent 1200 series, with PDA detector, scan range 190-400 nm. Mass spectrometer: Agilent MSD 6120 operating in electrospray ionisation mode with +ve/−ve ion switching.

LC Conditions.

Mobile phase A: 0.1% Formic acid in water

Mobile phase B: 0.1% Formic acid in acetonitrile

Gradient.

| Time (mins.) | % B |
|---|---|
| 0 | 2 |
| 0.3 | 2 |
| 2 | 95 |
| 2.45 | 95 |

Flow rate: 1.5 ml/min.

Column: Varian Pursuit Ultra 3 C18 30 mm×2.1 mm

Column temp: 50° C.

2. (QC Method 2)

Liquid Chromatograph: Agilent 1200 series, with PDA detector, scan range 190-400 nm.

Mass spectrometer: Agilent MSD 6120 operating in electrospray ionisation mode with +ve/ −ve ion switching.

LC Conditions.

Mobile phase A: 0.1% Formic acid in water

Mobile phase B: 0.1% Formic acid in acetonitrile

Gradient.

| Time (mins.) | % B |
|---|---|
| 1 | 5 |
| 5 | 95 |
| 1.45 | 95 |

Flow rate: 1.5 ml/min.

Column: Varian Pursuit Ultra 3 C18 30 mm×2.1 mm

Column temp: 50° C.

3. (QC Method 3)

Liquid Chromatograph: Agilent 1200 series, with PDA detector, scan range 190-400 nm.

Mass spectrometer: Agilent MSD 6120 operating in electrospray ionisation mode with +ve/−e ion switching.

LC Conditions.

Mobile phase A: 0.1% formic acid in water

Mobile phase B: 0.1% formic acid in acetonitrile

Gradient.

| Time (mins.) | % B |
|---|---|
| 2 | 5 |
| 6 | 95 |
| 4.9 | 95 |
| 7 | 5 |

is Flow rate: 1.0 ml/min.

Column: Varian Pursuit Ultra 3 C18 50 mm×2.1 mm

Column temp: 50° C.

4. (QC Method 4)

Liquid Chromatograph: Waters Acquity UPLC, with PDA detector, (scan range 190-400 nm) and ELSD.

Mass spectrometer: Waters SQD operating in electrospray ionisation mode with +ve/−ve ion switching.

LC Conditions.

Mobile phase A: 0.1% formic acid in water

Mobile phase B: 0.1% formic acid in acetonitrile

Gradient.

| Time (mins.) | % B |
|---|---|
| 3 | 5 |
| 0.2 | 5 |
| 4.5 | 95 |
| 8 | 95 |

Flow rate: 0.6 ml/min

Column: Waters Acquity UPLC BEH C18 50 mm×2.1 mm 1.7 um

Column temp: 50° C.

5. (QC Method 5)

Liquid Chromatograph: Waters Acquity UPLC, with PDA detector, (scan range 190-400 nm) and ELSD.

Mass spectrometer: Waters SQD operating in electrospray ionisation mode with +ve/−ve

ion switching.

LC Conditions.

Mobile phase A: 0.1% ammonia in water

Mobile phase B: 0.1% ammonia in acetonitrile

Gradient.

| Time (mins.) | % B |
|---|---|
| 4 | 5 |
| 0.2 | 5 |
| 4.5 | 95 |
| 9 | 95 |

Flow rate: 0.6 ml/min

Column: Waters Acquity UPLC BEH C18 50 mm×2.1 mm 1.7 um

Column temp: 50° C.;

(x) unless stated otherwise compounds containing an asymmetrically substituted carbon and/or sulfur atom have not been resolved;

(xi) all microwave reactions were carried out in a CEM Discover® microwave synthesiser;

(xii) Preparative high performance liquid chromatography (HPLC) was carried out using the following conditions, unless otherwise stated:

Liquid Chromatograph: Waters 600 pump, W2700 Sample Manager, W996 PDA detector

Mass spectrometer: Waters ZQ operating in electrospray ionisation mode.

LC Conditions.

Mobile phase A: 0.1% formic acid in water

Mobile phase B: 0.1% formic acid in acetonitrile

Gradient.

| Time (mins.) | % B |
|---|---|
| 5 | 5 |
| 15 | 75 |
| 16 | 100 |
| 18 | 100 |

Flow rate: 20 ml/min

Column: Gemini C18 50 mm×21.2 mm 5 um 110A Axia (Phenomenex Ltd);

(xiii) the following abbreviations have been used herein, where necessary:
DCM Dichloromethane
DMF N,N-Dimethylformamide
DMSO Dimethylsulfoxide
Ether Diethyl ether
00EtOAc Ethyl acetate
EtOH Ethanol
FCC Flash column chromatography
HBTU O-(1H-benzotriazol-1-yl)-N,N,N′,N′,-tetramethyluronium hexafluorophosphate
NBS N-Bromosuccinamide
PE Petroleum ether
PMB p-Methoxybenzyl
Rₜ Retention time
RT Room temperature
TEA Triethylamine
TFA Trifluoroacetic acid
THF Tetrahydrofuran

### EXAMPLES

### Synthesis of Intermediates

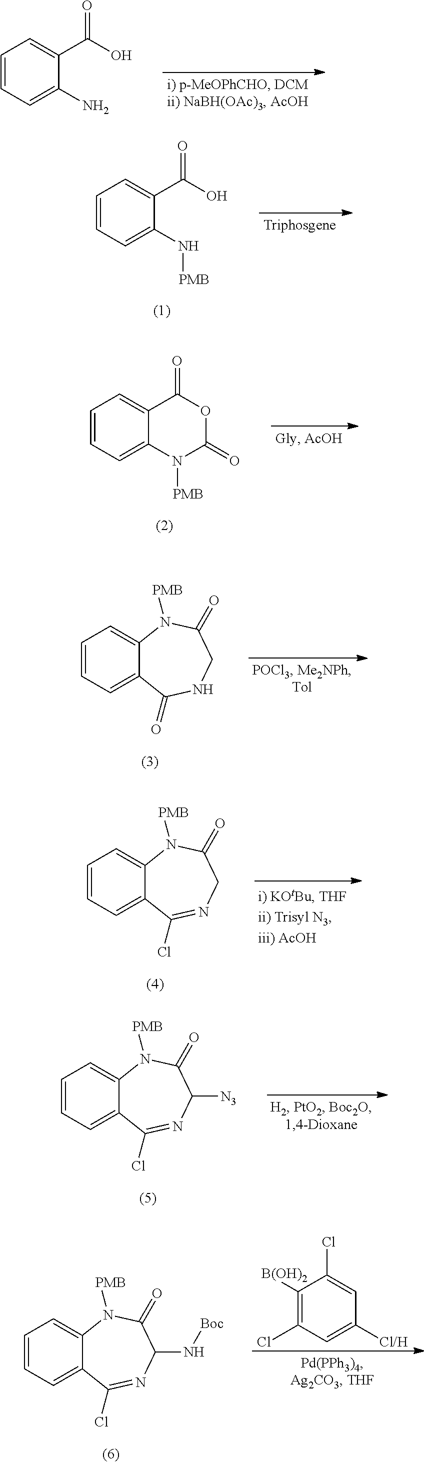

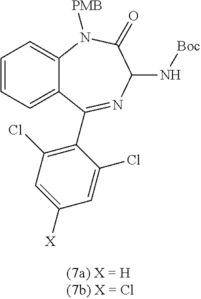

### 2-(4-Methoxy-benzylamino)benzoic acid (1)

A suspension of anthranilic acid (23.7 g, 173 mmol), p-methoxybenzaldehyde (26 ml, 214 mmol) and acetic acid (5 ml, 87 mMol) in DCM (500 ml) was stirred at RT for 2 hrs. The reaction mixture was cooled to 0° C. and sodium triacetoxyborohydride (78 g, 368 mmol) was added in a portionwise manner. On completion of addition the reaction was warmed to RT and stirred overnight.

The reaction was diluted with DCM and washed with water. The organics were dried and concentrated. The residue was triturated with ether to give the title compound; (41.2 g, 160 mmol)

NMR δ 7.89 (1H, dd, J 7.9, 1.6), 7.23-7.33 (3H, m), 6.89 (2H, m), 6.68 (1H, d, J 7.9 Hz), 6.54 (1H, m), 4.34 (2H, s), 3.72 (3H, s);

MS (m/e) 256 [M−H]⁻, Rₜ 0.92 min (QC Method 1)

### 1-(4-Methoxy-benzyl)-1H-benzo[d][1,3]oxazine-2,4-dione (2)

A solution of (1) (41.2 g, 160 mmol) in THF (400 ml) was treated portionwise with triphosgene (17.4 g, 59 mmol) and the resulting solution stirred at RT overnight.

The reaction was concentrated and the resulting solid triturated with ether to give the title compound; (40.1 g, 143 mmol)

NMR δ 8.01 (1H, dd, J 8.2, 1.9), 7.74 (1H, m), 7.25-7.37 (3H, m), 6.88 (2H, m), 5.21 (2H, s), 3.70 (3H, s);

MS (m/e) No MI observed, Rₜ 2.8 min (QC Method 3)

### 1-(4-Methoxy-benzyl)-3,4-dihydro-1H-benzo[e][1,4]diazepine-2,5-one (3)

A suspension of (2) (40.1 g, 143 mmol) and glycine (25.0 g, 333 mmol) in acetic acid (300 ml) was heated at reflux overnight.

The reaction was cooled and concentrated. The residue was partitioned between DCM and 1M HCl. The organics were separated, washed with saturated NaHCO₃ and brine and dried before being concentrated. The resulting solid was triturated with ether to give the title compound; (32.1 g, 108 mmol)

NMR δ 8.78 (1H, t, J 6.0), 7.65 (1H, dd, J 7.6, 1.3), 7.45-7.58 (2H, m), 7.28 (1H, ddd, J 8.2, 6.3, 1.9), 7.04 (2H, d, J 8.4), 6.81 (2H, d, J 8.4), 5.27 (1H, d, J 15.6), 4.88 (1H, d, J 15.6), 3.69 (3H, s);

MS (m/e) 297 [M+H]⁺, Rₜ 0.71 min (QC Method 2)

### 5-Chloro-1-(4-methoxy-benzyl)-1,3-dihydro-benzo[e][1,4]diazepin-2-one (4)

A suspension of (3) (11.1 g, 38 mmol) and N,N-dimethylaniline (9.5 ml, 75 mmol) in toluene (100 ml) was treated with phosphorus oxychloride (3.5 ml, 38 mmol) and the resulting solution heated at reflux overnight.

The reaction was cooled to RT, diluted with toluene and poured into ice-cold saturated K₂CO₃ solution (ca. 300 ml). The organics were separated and the aqueous backwashed with toluene. The combined organics were dried and concentrated to an oil. This was applied to a flash-silica column, preconditioned with 3% TEA in PE, and eluted with a PE to 1:3 EtOAc:PE gradient to give the title compound; (10.7 g, 34 mmol) NMR δ 7.71 (1H, dd, J 8.5, 0.9), 7.54-7.64 (2H, m), 7.27-7.35 (1H, m), 6.96 (2H, d, J 8.6). 6.79 (2H, d, J 8.6), 5.28 (1H, d, J 15.6), 4.88 (1H, d, J 15.6), 4.45 (1H, d, J 10.7), 3.86 (1H, d, J 10.7), 3.66 (3H, s);

MS (m/e) 315 [M+H]⁺, Rₜ 0.90 min (QC Method 2)

### 3-Azido-5-chloro-1-(4-methoxy-benzyl)-1,3-dihydro-benzo[e][1,4]diazepin-2-one (5)

A stirred solution of (4) (10.7 g, 34 mmol) in anhydrous THF (200 ml), under N₂, was cooled to −78° C. and treated dropwise with 1M potassium t-butoxide in THF (40 ml, 40 mmol). After 30 minutes a solution of trisyl azide (12.3 g, 40 mmol) in THF (60 ml ) was added. After a further 45 minutes acetic acid (30 ml, 525 mmol) was added and the reaction allowed to warm to RT and stirred overnight.

The reaction was concentrated and the residue portioned between EtOAc and saturated NaHCO₃ solution. The organics were separated, dried and concentrated to give an oil. This was applied to a flash-silica column, preconditioned with 3% TEA in PE, and eluted with a PE to 1:3 EtOAc:PE gradient to give the title compound; (7.4 g, 21 mmol) NMR δ 7.75 (1H, dd, J 8.5, 0.9), 7.62-7.68 (2H, m), 7.32-7.40 (1H, m), 6.96 (2H, d, J 8.6), 6.80 (2H, d, J 8.6), 5.33 (1H, d, J 15.5), 4.98 (1H, s), 4.95 (1H, d, J 16.4), 3.66 (3H, s);

MS (m/e) No MI observed, Rₜ 1.02 min (QC Method 2)

### [5-Chloro-1-(4-methoxy-benzyl)-2-oxo-2,3-dihydro-1H-benzo[e][1,4]diazepin-3-yl]-carbamic acid tert-butyl ester (6)

A solution of (5) (7.4 g, 21 mmol) in 1,4-dioxane (100 ml) was added to platinum (IV) oxide (0.74 g, 3.2 mmol) and di-tent-butyl dicarbonate (7.4 g, 34 mmol) and placed under an atmosphere of H₂ using standard hydrogenation procedures.

On completion, the reaction was filtered through Celite® and the filtrate concentrated to give an oil. This was applied to a flash-silica column, preconditioned with 3% TEA in PE, and eluted with a PE to 1:3 EtOAc:PE gradient to give the title compound; (6.6 g, 15 mmol)

NMR δ 7.98 (1H, d, J 8.8), 7.75 (1H, d, J 7.9), 7.62-7.71 (2H, m), 7.33-7.41 (1H, m), 6.92 (2H, d, J 8.5), 6.78 (2H, d, J 8.5), 5.37 (1H, d, J 15.5), 5.09 (1H, d, J 8.5), 4.89 (1H, d, J 15.5), 3.65 (3H, s), 1.37 (9H, s);

MS (m/e) 330 [(M-Boc)+H]⁺, Rₜ 1.06 min (QC Method 2) tert-Butyl 5-(2,6-dichlorophenyl)-1-(4-methoxybenzyl)-2-oxo-2,3-dihydro-1H-benzo[e][1,4]diazepin-3-ylcarbamate (7a)

A suspension of (6) (2.50 g, 5.8 mmol), 2,6-dichlorophenylboronic acid (1.66 g, 8.7 mmol), tetrakis(triphenylphosphine)palladium(0) (3.36 g, 2.9 mmol), K₂CO₃ (1.60 g, 11.6 mmol) and Ag₂CO₃ (8.00 g, 29 mmol) in THF (100 ml) was heated at reflux for 1 hr. The inorganics were removed by filtration and the filtrated reduced onto silica. Column chromatography (SiO₂; 4:1→1:1 PE:EtOAc) gave the title compound; (1.40 g, 2.6 mmol)

NMR δ 7.93 (1H, d, J 8.8), 7.63-7.79 (3H, m), 7.40-7.52 (2H, m), 7.17-7.32 (3H, m), 7.08 (1H, d, J 8.2), 6.84 (2H, d, J 8.2), 5.24 (1H, d, J 8.2), 5.18 (2H, s), 3.72 (3H, s), 1.41 (9H, s);

MS (m/e) 540 [MH]⁺, Rₜ 2.86 min (QC Method 3)

### [1-(4-Methoxy-benzyl)-2-oxo-5-(2,4,6-trichloro-phenyl)-2,3-dihydro-1H-benzo[e][1,4]diazepin-3-yl]-carbamic acid tert-butyl ester (7b)

A suspension of (6) (4.3 g, 10 mmol), 2,4,6-trichlorophenyl boroninc acid (4.3 g, 19 mmol), tetrakis(triphenylphospine)palladium (0) (4.6 g, 4 mmol), K₂CO₃ (3.0 g, 21 mmol) and Ag₂CO₃ (8.6 g, 31 mmol) in THF (120 ml) was heated at reflux for 72 hrs. The reaction was cooled and filtered through celite. The filtrate was reduced onto silica and column chromatography (SiO₂; PE→3:2 PE:ether) gave a partially pure sample. Further chromatograph (SiO₂; 19:1 DCM:MeOH) gave the title compound; (1.3 g, 2.2 mmol)

NMR δ 7.96 (1H, d, J 8.5), 7.77 (1H, d, J 8.2), 7.59-7.71 (2H, m), 7.05-7.30 (4H, m), 6.80 (2H, d, J 8.5), 5.04-5.25 (3H, m), 3.67 (3H, s), 1.38 (9H, s);

MS (m/e) 576 [M+H]⁺, Rₜ 1.23 min (QC Method 2)

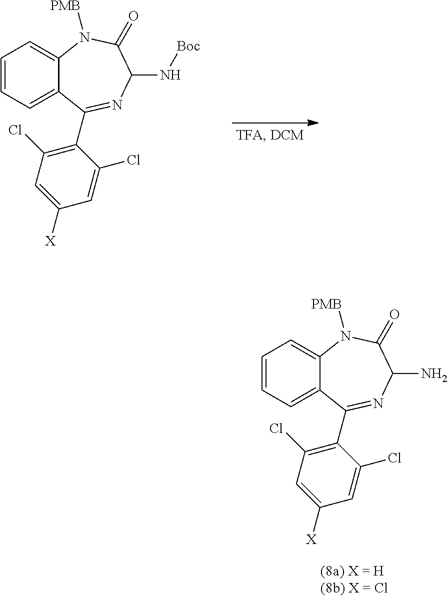

### 3-Amino-5-(2,6-dichlorophenyl)-1-(4-methoxybenzyl)-1H-benzo[e][1,4]diazepin-2(3H)-one (8a)

A solution of (7a) (1.40 g, 2.6 mmol) in DCM (50 ml) was treated with TFA (5 ml) and stirred for 3 hr. The reaction was diluted with DCM and poured into saturated NaHCO₃ solution to neutralise. The organics were separated, dried and concentrated to give the title compound; (1.14 g, 2.6 mmol)

MS (m/e) 440 [MH]⁺, Rₜ 1.97 min (QC Method 3)

### 3-Amino-1-(4-methoxy-benzyl)-5-(2,4,6-trichloro-phenyl)-1,3-dihydro-benzo[e][1,4]diazepin-2-one (8b)

A solution of (7b) (1.2, 2 mmol) in DCM (50 ml) was treated with TFA (5 ml) and stirred for 3 hrs. The reaction was diluted with DCM (100 ml) and then basified with saturated NaHCO₃ solution. The organics were separated, dried and concentrated to give the title compound; (0.91 g, 1.9 mmol)

NMR (CDCl₃) δ 7.42-7.50 (3H, m), 7.23-7.30 (3H, m), 7.16 (2H, d, J 8.5), 6.83 (2H, d J 8.5), 5.37 (1H, d, J 15.1), 4.90 (1H, d, J 15.1), 4.70 (1H, s), 3.79 (3H, s), 2.53 (2H, br);

MS (m/e) 476 [M+H]⁺, Rₜ 1.21 min (QC Method 3)

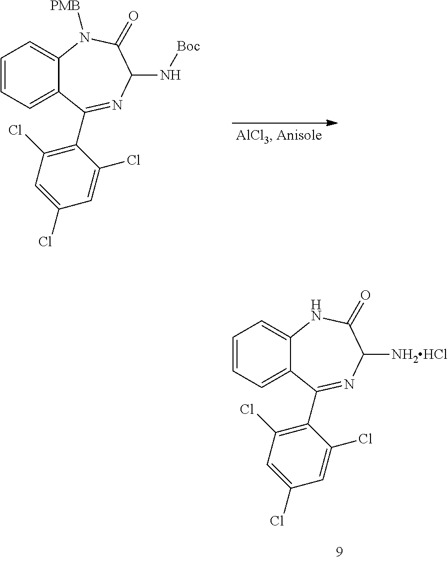

### 3-Amino-5-(2,4,6-trichloro-phenyl)-1,3-dihydro-benzo[e][1,4]diazepin-2-one hydrochloride (9)

A solution of (7b) (1.7 g, 3 mmol) in anhydrous anisole (25 ml) was treated with aluminium trichloride (4.0 g, 30 mmol) and then heated at 70° C. for 4 hrs.

The reaction was cooled to RT, diluted with EtOAc (200 ml) and basified with saturated K₂CO₃ solution. The resulting slurry was filtered through Celite® and the organics separated, dried and concentrated. The resulting residue was taken into DCM (50 ml) and stirred for 2 hrs with 2M HCl (50 ml). The resulting precipitate was filtered off and dried under vacuum to give the title compound; (0.82 g, 2.3 mmol)

NMR δ 11.54 (1H, s), 9.09 (3H, br, s), 7.95 (1H, d, J 1.9), 7.77 (1H, d, J 1.9), 7.59-7.72 (1H, m), 7.34 (1H, d, J 7.9), 7.14-7.26 (2H, m), 5.27 (1H, s);

MS (m/e) 356 [M+H]⁺, Rₜ 0.69 min (QC Method 2)

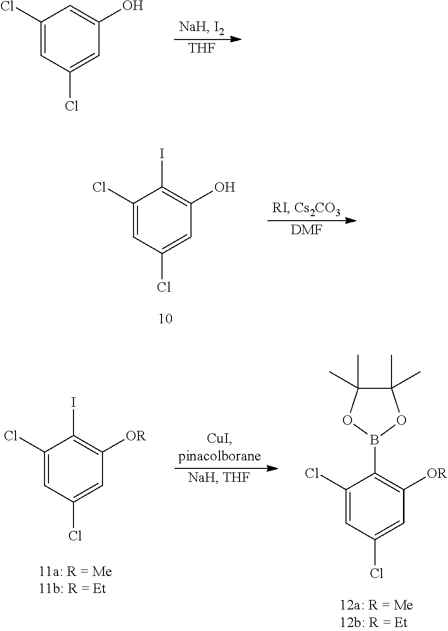

### 3,5-Dichloro-2-iodophenol (10)

To a solution of 3,5-dichlorophenol (30 g, 184 mmol) in toluene (600 ml) at 0° C. was slowly is added NaH (60% dispersion in mineral oil, 14.7 g 368 mmol). The reaction mixture was warmed to room temperature and stirred for 20 min before cooling back to 0° C. I₂ (49 g, 193 mmol) was then added and the reaction mixture was stirred at room temperature for 16 h before quenching with 2M HCl (500 ml). The organic materials were then extracted into Et₂O, washed with brine, dried and then concentrated to provide the crude product which was used without further purification;

NMR (CDCl₃) δ 7.14 (1H, d, J 2.2), 6.97 (1H, d, J 2.2), 5.68 (1H, br s);

MS (m/e) 287 [M−H]⁻, Rₜ 0.98 min (QC Method 2)

### 1,5-Dichloro-2-iodo-3-methoxybenzene (11a)

To the crude material from the preparation of (10) in DMF (300 ml ) was added CS₂CO₃ (63.7 g, 196 mmol) and MeI (14.4 ml, 231 mmol). After 16 h, the reaction mixture was filtered over Celite, concentrated, and then partitioned between EtOAc and 2M HCl. Separation of the organic phase and concentration provided an oil, which was triturated from PE to provide the title compound as a pale yellow solid; (32 g, 57%) NMR (CDCl₃) δ 7.19 (1H, d, J 2.2), 6.74 (1H, d, J 2.2), 3.95 (3H, s);

MS (m/e) No MI observed, Rₜ 1.15 min (QC Method 2).

### 1,5-Dichloro-2-iodo-3-ethoxybenzene (11b)

Using an analogous method to the preparation of 11a, the title boronate was prepared as a white solid; (35 g, 60%)

NMR (CDCl₃) δ 7.12 (1H, d, J 2.1), 6.66 (1H, d, J 2.1), 4.10 (2H, q, J 7.0), 1.53 (3H, t, J 7.0)

### 2-(2,4-Dichloro-6-methoxyphenyl)-4,4,5,5-tetramethyl-1,3,2-dioxaborolane (12a)

To a solution of 11a (21.0 g, 69.3 mmol) in THF (250 ml) was sequentially added CuI (1.32 g, 6.9 mmol) and NaH (60% dispersion in mineral oil, 4.2 g, 104 mmol), followed by a slow addition of pinacolborane (15.1 ml, 104 mmol). The resulting suspension was stirred at room temperature for 16 h under a N₂ atmosphere, and then quenched with saturated

NH₄Cl (250 ml). After 20 min, the mixture was extracted with EtOAc (×3), dried and then filtered over Celite. Concentrated and purification by column chromatography (SiO₂; EtOAc:PE 0:1→3:7) to afford the title boronate as a white solid; (15.1 g, 72%) NMR (CDCl₃) 6.87 (1H, d, J 1.6), 6.63 (1H, d, J 1.6), 3.37 (3H, s), 1.32 (12H, s); MS (m/e) No MI observed, Rₜ 1.17 min (QC Method 2)

### 2-(2,4-Dichloro-6-ethoxyphenyl)-4,4,5,5-tetramethyl-1,3,2-dioxaborolane (12b)

Using an analogous method to the preparation of 12a, 11b (23.5 g, 74.1 mmol) provided the title boronate as a white solid; (18.5 g, 79%)

NMR (CDCl₃) δ 6.96 (1H, d, J 1.6), 6.70 (1H, d, J 1.6), 4.00 (2H, q, J 7.0), 1.45-1.38 (3H, m), 1.42 (12H, s);

MS (m/e) No MI observed, Rₜ 1.22 min (QC Method 2)

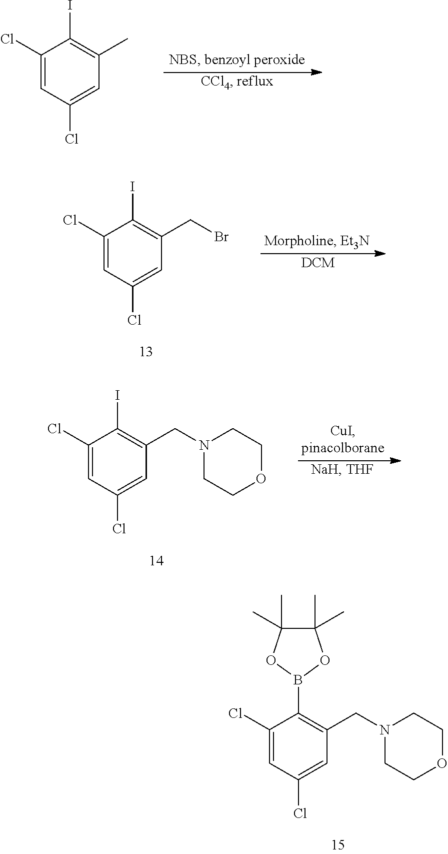

### 1-(Bromomethyl)-3,5-dichloro-2-iodobenzene (13)

3,5-Dichloro-2-iodotoluene (12.5 g, 43.6 mmol), NBS (11.7 g, 66 mmol) and benzoyl peroxide (1.1 g, 4.4 mmol) in CCl₄ were heated at 100° C. for 6 d under N₂. After cooling to room temperature, the suspension was filtered and concentrated. The mixture was then diluted with DCM, washed with water, and then concentrated. The crude product could be used without further purification, or can be purified by column chromatography (SiO₂; is EtOAc:PE 0:1→1:49) to afford the title bromide as a white solid; (6.7 g, 42%)

NMR (CDCl₃) δ 7.44 (1H, dd, J 2.2, 6.6), 7.40-7.38 (1H, m), 4.64 (1H, s), 4.60 (1H, s); MS (m/e) No MI observed, Rₜ 1.19 min (QC Method 2).

### 4-(3,5-Dichloro-2-iodobenzyl)morpholine (14)

The crude material from preparation of 14 (theoretical yield 15.9 g, 43.5 mmol) was dissolved in DCM (150 ml) and treated with TEA (12.1 ml, 87 mmol) and morpholine (7.5 ml, 87 mmol). After 16 h, the reaction mixture was washed with water, dried, concentrated and purified by column chromatography (SiO₂; EtOAc:PE 0:1→1:4) to afford the title compound as a white solid; (7.6 g, 47% over 2 steps)

NMR (CDCl₃) δ 7.50-7.28 (2H, m), 3.82-3.72 (4H, m), 3.61 (1H, s), 3.56 (1H, s), 2.60-2.52 (4H, m)

### 4-(3,5-Dichloro-2-(4,4,5,5-tetramethyl-1,3,2-dioxaborolan-2-yl)benzyl)morpholine (15)

To a solution of 14 (7.6 g, 20.3 mmol) in THF (75 ml) was sequentially added CuI (0.39 g, 2.0 mmol) and NaH (60% dispersion in mineral oil, 1.2 g, 31 mmol), followed by a slow addition of pinacolborane (4.4 ml, 31 mmol). The resulting suspension was stirred at room temperature for 3 days under a N₂ atmosphere, and then quenched with saturated NH₄Cl (750 ml). After 20 min, the mixture was extracted with EtOAc (×3), dried and then filtered over Celite. Concentrated and purification by column chromatography (SiO₂; EtOAc:PE 0:1→2:3) to afford the title boronate as a white solid; (3.5 g, 46%)

NMR (CDCl₃) δ 7.17 (1H, d, J 1.9), 7.09 (1H, d, J 1.9), 3.67-3.60 (4H, m), 3.45 (2H, s), 2.41-2.34 (4H, m), 1.35 (12H, s)

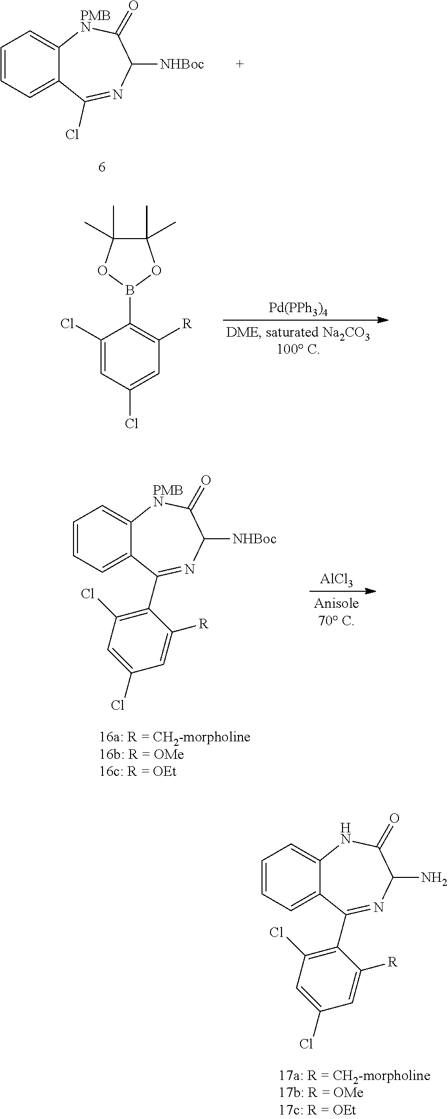

### tert-Butyl 5-(2,4-dichloro-6-(morpholinomethyl)phenyl)-1-(4-methoxybenzyl)-2-oxo-2,3-dihydro-1H-benzo [e][1,4]diazepin-3-ylcarbamate (16a)

A solution of 6 (2.35 g, 5.5 mmol), boronate 15 (1.44 g, 6.6 mmol) and Pd(PPh₃)₄ (0.63 g, 0.6 mmol) in 1,2-dimethoxyethane (50 ml) and saturated Na₂CO₃ (25 ml) were heated at 100° C. for 16 h. The reaction mixture was cooled to room temperature, diluted with water, extracted with EtOAc (×3), and dried. Concentration and purification by column chromatography (SiO₂; EtOAc:PE 0:1→1:1) provided the title compound as a pale yellow foam; (2.43 g, 69%)

NMR (CDCl₃) δ 7.50-7.36 (3H, m), 7.24-7.19 (1H, m), 7.15-7.09 (1H, m), 6.88-6.81 (2H, m), 6.52-6.45 (1H, m), 5.43 (1H, d, J 8.8), 5.31 (1H, d, J 15.3), 4.86 (1H, d, J 15.3), 4.04 (1H, d, J 13.8), 3.80 (3H, s), 3.69-3.50 (4H, m), 3.31 (1H, d, J 13.8), 2.62-2.41 (4H, m), 1.46 (9H, s);

MS (m/e) 639 [M+H]⁺, Rₜ 0.95 min (QC Method 2)

### tert-Butyl 5-(2,4-dichloro-6-methoxyphenyl)-1-(4-methoxybenzyl)-2-oxo-2,3-dihydro-1H-benzo [e][1,4]diazepin-3-ylcarbamate (16b)

A solution of 6 (16.0 g, 37 mmol), boronate 12a (13.0 g, 43 mmol) and Pd(PPh₃)₄ (3.7 g, 3.2 mmol) in 1,2-dimethoxyethane (120 ml) and saturated Na₂CO₃ (60 ml) were heated at 100° C. for 2 h. The reaction mixture was cooled to room temperature, diluted with water, extracted with EtOAc (×3), and dried. Concentration and trituration with ether provided the title compound as a white solid. (11.8 g) Concentration of the mother liquor followed by purification by column chromatography (SiO₂; EtOAc:PE 0:1→2:3) provided a further crop of the desired product as a white solid; (7.5 g, combined mass 19.3 g, total yield 91%)

NMR δ 8.01-7.80 (0.3H, m), 7.70-7.44 (1.7H, m), 7.41-7.04 (6H, m), 6.92-6.77 (2H, m), 5.25-4.95 (2H, m), 3.86 (1.7H, s), 3.70 (1.3H, s), 3.69 (1.7H, s), 3.35 (1.3H, s), 1.38 (7.8H, s), 1.31 (1.2H, s);

MS (m/e) 570 [M+H]⁺, Rₜ 1.19 min (QC Method 2)

### tert-Butyl 5-(2,4-dichloro-6-ethoxyphenyl)-1-(4-methoxybenzyl)-2-oxo-2,3-dihydro-1H-benzo [e][1,4]diazepin-3-ylcarbamate (16c)

A solution of 6 (16.2 g, 38 mmol), boronate 12b (13.1 g, 42 mmol) and Pd(PPh₃)₄ (1.3 g, 1.1 mmol) in 1,2-dimethoxyethane (160 ml) and saturated Na₂CO₃ (80 ml) were heated at 100° C. After 2 h, additional Pd(PPh₃)₄ (0.04 g, 0.3 mmol) was added and the reaction mixture heated for a further 1 h. After cooling to room temperature, the mixture was diluted with water, extracted with EtOAc (×3), and dried. Concentration and trituration with ether provided the title compound as a white solid. (13.4 g) Concentration of the mother liquor followed by purification by column chromatography (SiO₂; EtOAc:PE 0:1→2:3) provided another crop of the desired product as a white solid; (3.8 g, combined mass 17.2 g, total yield 78%)

NMR δ 7.90-7.78 (0.4H, m), 7.71-7.50 (1.1H, m), 7.39-7.05 (6.4H, m), 6.96-6.77 (2.1H, m), 5.43-5.09 (1.8H, m), 4.61 (0.2H, d, J 15.8), 4.23-4.01 (0.8H, m), 3.84-3.65 (1.2H, m), 3.74 (1.2H, s), 3.68 (1.8H, s), 1.39 (9H, s), 1.20 (1.8H, t, J 7.0), 0.78 (1.2H, t, J 7.0);

MS (m/e) 584 [M+H]⁺, Rₜ 1.23 min (QC Method 2)

### General Method A: Double Deprotection of Suzuki Product:

To tert-butyl 5-aryl-1-(4-methoxybenzyl)-2-oxo-2,3-dihydro-1H-benzo[e][1,4]diazepin-3-ylcarbamate (16) (5.8 mmol) in anisole (30 ml) was added AlCl₃ (28.8 mmol) and the solution heated to 70° C. under N₂ for 16 h. Upon cooling to room temperature, the reaction mixture was slowly added to saturated NaHCO₃ (200 mL). Celite was added to the suspension, which was then filtered over Celite, washing with copious quantities of a mixture of EtOAc and acetone. The aqueous phase from the filtrate was subsequently removed, and the organic materials were extracted with EtOAc (×2). The combined organic extracts were dried, concentrated and triturated with ether to provide the doubly deprotected material.

### 3-Amino-5-(2,4-dichloro-6-(morpholinomethyl)phenyl)-1H-benzo [e][1,4]diazepin-2(3H)-one (17a)

Following General Method A, 16a (1.0 g, 1.6 mmol) provided 17a as a pale brown powder. (0.4 g, 61%)

NMR: Peaks too broad to assign;

MS (m/e) 419, [M+H]⁺, Rₜ 0.58 min (QC Method 2).

### 3-Amino-5-(2,4-dichloro-6-methoxyphenyl)-1H-benzo [e] [1,4] diazepin-2(3H)-one (17b)

Following General Method A, 16b (4.0 g, 7.0 mmol) provided 17b as a pale yellow powder. (2.0 g, 81%)

NMR δ 10.77 (1H, br s), 7.53-7.44 (1H, m), 7.35 (0.3H, d, J 1.6), 7.30 (0.7H, d, J 1.6), 7.20-7.02 (4H, m), 4.27 (1H, m), 3.91 (2H, m), 3.50 (1H, m);

MS (m/e) 350 [M+H]⁺, Rₜ 0.62 min (QC Method 2).

### 3-Amino-5-(2,4-dichloro-6-ethoxyphenyl)-1H-benzo [e][1,4]diazepin-2(3H)-one (17c)

Following General Method A, 16c (3.4 g, 5.8 mmol) provided 17c as a pale grey powder. (1.5 g, 71%)

NMR δ 10.81 (0.6H, s), 10.77 (0.4H, s), 7.53-7.42 (1H, m), 7.34-7.27 (1H, m), 7.21-7.04 (4H, m), 4.30-4.12 (1.8H, m), 3.91-3.75 (0.6H, m), 3.70-3.54 (0.6H, m), 1.28 (1.2H, t, J 7.0), 0.81 (1.8H, t, J 7.0);

MS (m/e) 364 [M+H]⁺, Rₜ 0.65 min (QC Method 2).

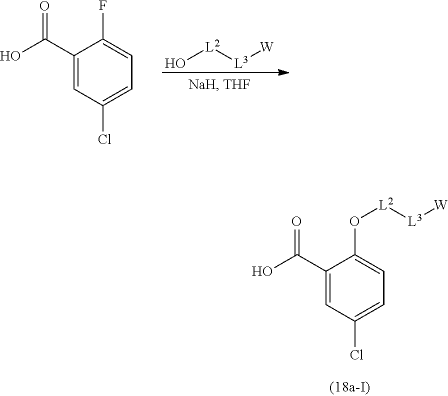

### 5-Chloro-2-(2-methoxy-propoxy)-benzoic acid (18a)

A solution of 2-fluoro-5-chlorobenzoic acid (2.9 g, 16.6 mmol) and 2-methoxypropanol (4.5 g, 50 mmol) in anhydrous THF (125 ml) was treated portionwise with NaH (60% dispersion in mineral oil, 5.0 g, 125 mmol). The resulting mixture was stirred at reflux for 24 hrs.

The reaction was cooled to RT, diluted with EtOAc and acidified with 2M HCl.

The organics were separated, dried and concentrated to give an oil. Column chromatography (SiO₂; 1:0→1:1 PE:EtOAc) gave the title compound (1.42 g, 5.8 mmol)

NMR (CDCl₃) δ 8.16 (1H, d, J 2.8), 7.49 (1H, dd, J 8.8, 2.8), 7.00 (1H, d, J 8.8), 4.35 (2H, m), 3.57 (2H, m), 3.39 (3H, s), 2.17 (2H, m)

### 5-Chloro-2-(2-(S)-methoxy-propoxy)-benzoic acid (18b)

Synthesis analogous to 18a, using 2-(S)-methoxy-propanol

NMR (CDCl₃) δ 8.10 (1H, d, J 2.8), 7.43 (1H, dd, J 8.8, 2.8), 6.98 (1H, d, J 8.8), 4.26 (1H, dd, J 9.5, 3.2), 4.02 (1H, m), 3.80 (1H, m), 3.42 (3H, s), 1.28 (3H, d, J 6.3)

### 5-Chloro-2-(2-ethoxyethoxy)benzoic acid (18c)

Synthesis analagous to 18a, using 2-ethoxyethanol

NMR (CDCl₃) δ 8.16 (1H, d, J 2.5), 7.52 (1H, dd, J 8.5, 2.5), 7.03 (1H, d, J 8.5), 4.37 (2H, t, J 4.4), 3.86 (J 4.4), 3.63 (2H, q, J 7.0), 1.28 (3H, t, J 7.0);

MS (m/e) 243 [M−H]⁻, Rₜ 1.41 min (QC Method 1)

### 5-Chloro-2-(1-methoxypropan-2-yloxy)benzoic acid (18d)

Synthesis analogous to 18a, using 1-methoxypropan-2-ol

NMR δ 7.57 (1H, d, J 2.8), 7.46 (1H, dd, J 8.8, 2.8), 7.18 (1H, d, J 8.8), 4.60 (1H, m), 3.37-3.52 (2H, m), 3.26 (3H, s), 1.19 (3H, d, J 6.3);

MS (m/e) 243 [M−H]⁻, Rₜ 1.41 min (QC Method 1)

### 5-Chloro-2-[2-(2-methoxy-ethoxy)-ethoxy]-benzoic acid (18e)

Synthesis analogous to 18a, using 2-(2-methoxyethoxy)ethanol

NMR (CDCl₃) δ 7.61 (1H, d, J 2.8), 7.03 (1H, dd, J 8.8, 2.8), 6.27 (1H, d, J 8.8), 3.95 (2H, m), 3.76 (2H, m), 3.62 (2H, m), 3.47 (2H, m), 3.19 (3H, s)

### 5-Chloro-2-(2-isobutoxyethoxy)benzoic acid (180

Synthesis analogous to 18a, using 2-isobutoxyethanol

NMR (CDCl₃) δ 7.61 (1H, d, J 2.5), 7.53 (1H, d, J 8.8, 2.8), 7.19 (1H, d, J 8.8), 4.17 (2H, m), 3.70 (2H, m), 3.24 (2H, d, J 6.6), 1.78 (1H, m), 0.84 (6H, d, J 6.6)

### 5-Chloro-2-(2-(pentyloxy)ethoxy)benzoic acid (18 g)

Synthesis analogous to 18a, using 2-pentlyoxyethanol

NMR (CDCl₃) δ 7.61 (1H, d, J 2.5), 7.51 (1H, dd, J 9.2, 2.8), 7.17 (1H, d, J 9.2), 4.16 (2H, m), 3.69 (2H, m), 1.19-1.32 (6H, m), 0.80-0.90 (5H, m)

### 5-Chloro-2-(2-(2-ethoxyethoxy)ethoxy)benzoic acid (18 h)

Synthesis analogous to 18a, using 2-(2-ethoxyethoxy)ethanol

NMR (CDCl₃) δ 7.62 (1H, d, J 8.2), 7.49 (1H, dd, J 8.8, 2.8), 7.15 (1H, d, J 9.2), 4.15 (2H, m), 3.74 (2H, m), 3.43-3.54 (4H, m), 1.09 (2H, t, J 7.0), 1.08 (3H, t, J 7.0)

### 5-Chloro-2-((tetrahydrofuran-2-yl)methoxy)benzoic acid (18i)

Synthesis analogous to 18a, using (tetrahydrofuran-2-yl)methanol

NMR (CDCl₃) δ 8.13 (1H, d, J 2.8), 7.51 (1H, dd, J 8.8, 2.8), 7.03 (1H, d, J 8.8), 4.29-4.40 (2H, m), 3.78-4.16 (3H, m), 1.63-2.21 (4H, m);

MS (m/e) 255 [M−H]⁻, Rₜ 1.40 min (QC Method 1)

### 2-(2-Butoxyethoxy)-5-chlorobenzoic acid (18j)

Synthesis analogous to 18a, using 2-butoxyethanol

NMR (CDCl₃) δ 8.15 (1H, d, J 2.8), 7.51 (1H, dd, J 8.8, 2.8), 7.04 (1H, d, J 8.8), 4.38 (2H, m), 3.85 (2H, m), 3.56 (2H, t, J 6.6), 1.63 (2H, m), 1.39 (2H, m), 0.94 (3H, t, J 7.3);

MS (m/e) 273 [M+H]⁺, Rₜ 0.87 min (QC Method 2)

### 5-Chloro-2-(2-phenoxyethoxy)benzoic acid (18k)

Synthesis analogous to 18a, using 2-phenoxyethanol

NMR δ 7.59 (1H, d, J 2.8), 7.54 (1H, dd, J 8.8, 2.8), 7.20-7.32 (3H, m), 6.89-6.99 (3H, m), 4.37 (2H, m), 4.28 (2H, m);

MS (m/e) 291 [M−H]⁻, Rₜ 1.62 min (QC Method 1)

### 5-Chloro-2-(2-(2-(dimethylamino)ethoxy)ethoxy)benzoic acid (181)

Synthesis analogous to 18a, using 2-(2-(dimethylamino)ethoxy)ethanol

MS (m/e) 288 [M+H]⁺, Rₜ 0.44 min (QC Method 2)

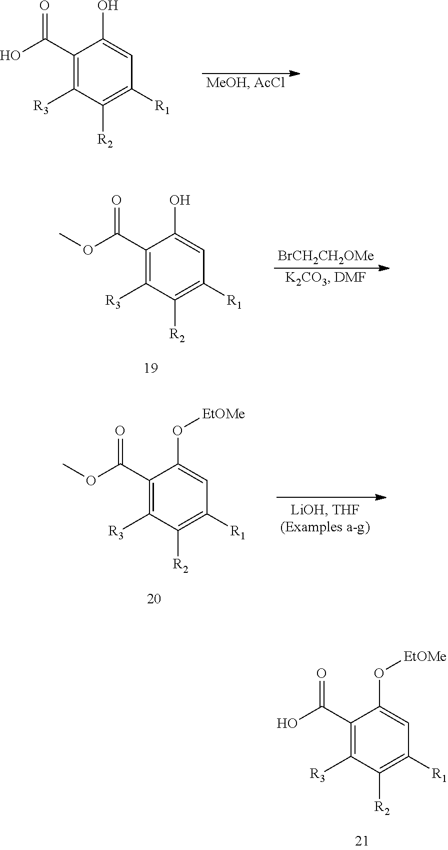

General Method B: 2-(Methoxyethoxy)-benzoic Acids from Salicylic Acids

### Exemplified by:

### Methyl 5-chlorosalicylate (19a)

A solution of 5-chlorosalicylic acid (75.0 g, 0.44 mol) in MeOH (250 ml) was treated portionwise with acetyl chloride (5 ml, 70 mmol) and heated at 90° C. for 3 d. The reaction mixture was chilled to −18° C. and the resulting precipitate collected by filtration. Further purification by trituration with 1:1 ether:PE ether gave the title compound; (71.99 g, 038 mmol)

NMR (CDCl₃) δ 10.70 (1H, s), 7.82 (1H, d, J 2.8), 7.42 (1H, dd, J 8.8, 2.8), 6.95 (1H, d, J 8.8)

### 5-Chloro-2-(2-methoxyethoxy)-benzoic acid methyl ester (20a)

A solution of 19a (10.0 g, 53.6 mmol) in DMF (250 ml) was treated with K₂CO₃ (19.5 g, 140 mmol) and 2-bromoethyl methyl ether (16.7 g, 120 mmol) and stirred at 90° C. overnight.

The reaction was cooled to RT, concentrated and the residue partitioned between water and DCM. The organics were separated, dried and concentrated. Column chromatography (SiO₂; 4:1 PE:ether) gave the title compound; (11.05 g, 45 mmol)

NMR (CDCl₃) δ 7.77 (1H, d, J 2.8), 7.39 (1H, dd, J 8.8, 2.8), 6.94 (1H, d, J 8.8), 4.17 (2H, m), 3.88 (3H, s), 3.78 (2H, m), 3.46 (3H, s)

### 5-Chloro-2-(2-methoxyethoxy)-benzoic acid (21a)

A solution of 20a (11.05 g, 45 mmol) in THF (200 ml) was treated with a 1M LiOH (75 ml, 75 mmol) and stirred for 48 hrs.

The reaction was diluted with ether and the organics separated. The aqueous was acidified with conc. HCl and extracted with DCM. The organics were dried and concentrated to give the title compound; (9.89 g, 43 mmol)

NMR (CDCl₃) δ 10.70 (1H, br), 8.10 (1H, d, J 2.5), 7.48 (1 h, dd, J 8.8, 2.5), 7.01 (1H, d, J 8.8), 4.34 (2H, m), 3.79 (2H, m), 3.45 (3H, s);

MS (m/e) 229 [M−H]⁻, Rₜ 1.30 min (QC Method 1)

### 2-(2-Methoxyethoxy)benzoic acid (21b)

Synthesised according to General Method B, using salicylic acid

NMR (CDCl₃) δ 11.10 (1H, br, s), 8.19 (1H, dd, J 8.9, 1.9), 7.57 (1H, m), 7.17 (1H, m), 7.07 (1H, dd, J 8.2, 0.9), 4.40 (2H, m), 3.83 (2H, m), 3.48 (3H, s)

### 5-Fluoro-2-(2-methoxyethoxy)benzoic acid (21c)

Synthesised according to General Method B, using 5-fluorosalicylic acid

NMR (CDCl₃) δ 7.29-7.44 (2H, m), 7.16 (1H, dd, J 9.2, 4.4), 4.15 (2H, m), 3.66 (2H, m), 3.32 (3H, s)

### 2-(2-Methoxyethoxy)-5-methylbenzoic acid (21d)

Synthesised according to General Method B, using 5-methylsalicylic acid

NMR (CDCl₃) δ 11.00 (1H, br), 7.97 (1H, d, J 2.2 Hz), 7.34 (1H, dd, J 8.5, 2.2), 6.96 (1H, d, J 8.5), 4.35 (2H, m), 3.79 (2H, m), 3.46 (3H, s), 2.34 (3H, s)

### 5-Acetyl-2-(2-methoxyethoxy)benzoic acid (21e)

Synthesised according to General Method B, using 5-acetyl-2-hydroxybenzoic acid

NMR (CDCl₃) δ 8.72 (1H, d, J 2.5), 8.21 (1H, dd, J 8.5, 2.5), 7.15 (1H, d, J 8.8), 4.46 (2H, m), 3.86 (2H, m), 3.49 (3H, s), 2.63 (3H, s)

### 2-(2-Methoxyethoxy)-5-(trifluoromethoxy)benzoic acid (21f)

Synthesised according to General Method B, using 5-(trifluoromethoxy)salicylic acid

NMR (CDCl₃) δ 8.06 (1H, d, J 3.2), 7.43 (1H, dd, J 8.8, 3.2), 7.12 (1H, d, J 8.8), 4.41 (2H, m), 3.84 (2H, m), 3.50 (3H, s)

### 5-Bromo-2-(2-methoxyethoxy)benzoic acid (21 g)

Synthesised according to General Method B, using 5-bromosalicylic acid

NMR (CDCl₃) δ 8.27 (1H, d, J 2.8), 7.65 (1H, dd, J 8.8, 2.8), 6.97 (1H, d, J 8.8), 4.37 (2H, m), 3.82 (2H, m), 3.48 (1H, s)

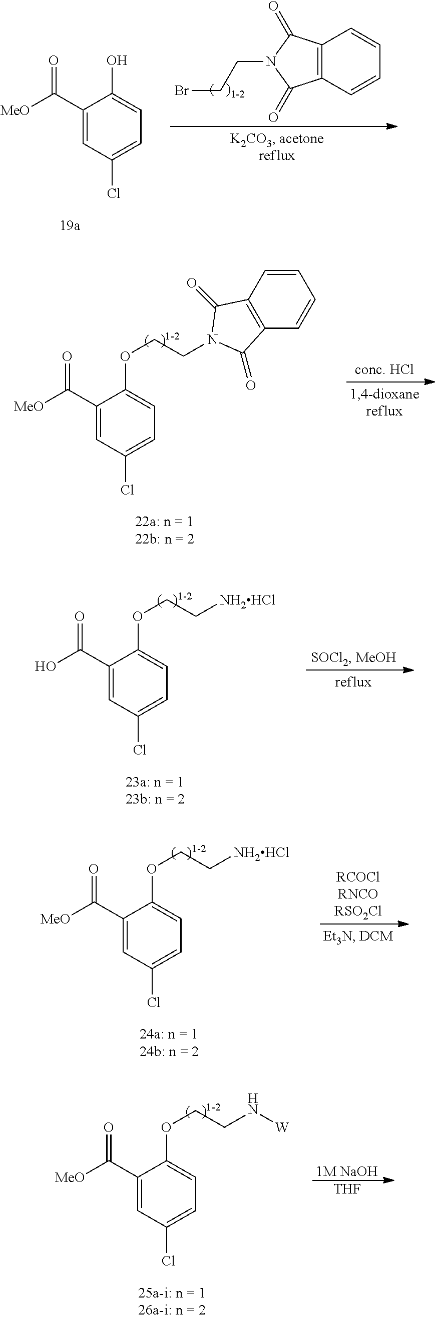

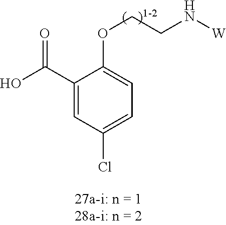

### Methyl 5-chloro-2-(2-(1,3-dioxoisoindolin-2-yl)ethoxy)benzoate (22a)

To a stirring solution of 19a (20.0 g, 110 mmol) in acetone (200 ml) was added K₂CO₃ (22.2 g, 160 mmol) and N-(2-bromoethyl)phthalimide (35.4 g, 140 mmol), and the suspension was heated at reflux for 5 d. The mixture was cooled to RT, concentrated, and then filtered, washing the cake thoroughly with DCM (500 ml). The filtrate was concentrated and purified by column chromatography (SiO₂; EtOAc:PE 1:4→1:1) to afford the title phthalimide as a white solid; (14.2 g, 37%)

NMR (CDCl₃) δ 7.82-7.71 (2H, m), 7.67-7.54 (3H, m), 7.28 (1H, dd, J 8.8, 2.5), 6.83 (1H, d, J 8.8), 4.22 (2H, t, J 5.7), 4.02 (2H, t, J 5.7), 3.72 (3H, s);

MS (m/e) No MI observed, Rₜ 0.95 min (QC Method 2)

### Methyl 5-chloro-2-(3-(1,3-dioxoisoindolin-2-yl)propoxy)benzoate (22b)

To a stirring solution of methyl 19a (10.0 g, 54 mmol) in acetone (100 ml) was added K₂CO₃ (14.8 g, 107 mmol) and N-(3-bromopropyl)phthalimide (18.7 g, 70 mmol), and the suspension was heated at reflux for 3 days. The mixture was cooled to RT, and then filtered, washing with acetone (2×100 ml). The cake was partitioned between DCM (150 ml) and water (150 ml) then the organic layer was removed, backwashing the aqueous phase with further DCM (150 ml). The combined organic extracts were dried to afford the title phthalimide as a white solid; (14.1 g, 71%)

NMR (CDCl₃) δ 7.87-7.71 (5H, m), 7.40 (1H, dd, J 8.8, 2.8), 6.91 (1H, d, J 8.8), 4.12 (2H, t, J 6.0), 3.97 (2H, t, J 7.0), 3.92 (3H, s), 2.30-2.20 (2H, m);

MS (m/e) No MI observed, Rₜ 0.95 min (QC Method 2)

### 2-(2-aminoethoxy)-5-chlorobenzoic acid hydrochloride (23a)

A suspension of phthalimide 22a (14 g, 39 mmol) in 1,4-dioxane (100 ml ) and concentrated HCl (50 ml) was heated at reflux for 3 days. Additional concentrated HCl (50 ml) was added and the mixture heated for a further 1 d. Concentration followed by trituration in acetone afforded the title hydrochloride salt as an off-white solid (8.4 g, 86%)

NMR δ 8.16 (3H, br s), 7.69 (1H, d, J 2.8), 7.61 (1H, dd, J 8.8, 2.8), 7.27 (1H, d, J 8.8), 4.29 (2H, t, J 5.4), 3.28-3.13 (3H, m);

MS (m/e) No MI observed, Rₜ 0.20 min (QC Method 2)

### 2-(3-aminopropoxy)-5-chlorobenzoic acid hydrochloride (23b)

A suspension of phthalimide 22b (30 g, 80 mmol) in 1,4-dioxane (150 ml) and concentrated HCl (80 ml) was heated at reflux for 5 days. Additional concentrated HCl (80 ml) was added and the mixture heated for a further 2 days. Concentration followed by trituration in acetone afforded the title hydrochloride salt as an off-white solid (15 g, 71%); NMR δ 8.01 (3H, br s), 7.70 (1H, d, J 2.8), 7.58 (1H, dd, J 8.8, 2.8), 7.19 (1G, d, J 8.8), 4.21 (2H, t, J 5.7), 3.12-3.01 (2H, m), 2.12-2.02 (2H, m);

MS (m/e) No MI observed, Rₜ 0.39 min (QC Method 2)

### Methyl 2-(2-aminoethoxy)-5-chlorobenzoate hydrochloride (24a)

SOCl₂ (7.3 ml, 100 mmol) was added dropwise to a solution of carboxylic acid 23a (8.4 g. 33 mmol) in MeOH (70 ml) at 0° C. The resulting mixture was heated at 60° C. for 16 hr, then cooled to room temperature and concentrated to afford methyl ester 24a as a white solid; (8.9 g, quantitative)

NMR δ 8.14 (3H, br s), 7.71 (1H, d, J2.8), 7.65 (1H, dd, J 8.8, 2.8), 7.28 (1H, d, J 8.8), 4.28 (2H, t, J 5.4), 3.83 (3H, s), 3.21 (2H, t, J 5.4);

MS (m/e) 230 [M+H]⁺, Rₜ 0.49 min (QC Method 2)

### Methyl 2-(3-aminopropoxy)-5-chlorobenzoate hydrochloride (24b)

Synthesis analogous to 24a, carboxylic acid 23b (15 g, 55 mmol) afforded methyl ester 24b as a white solid; (16 g, quantitative)

NMR δ 7.94 (3H, br s), 7.69 (1H, d, J 2.8), 7.58 (1H, dd, J 8.8, 2.8), 7.19 (1H, d, J 8.8), 4.17 (2H, t, J 5.8), 3.82 (3H, s), 3.06-2.91 (2H, m), 2.10-1.99 (2H, m);

MS (m/e) 244 [M+H]⁺, Rₜ 0.54 min (QC Method 2)

### General Method C: Preparation of Amido-Benzoates

A solution of the amine hydrochloride salt (3.6 mmol) in DCM (10 ml) was treated with TEA (8.9 mmol) and the acyl chloride (5.4 mmol). After 4 hr, 1M HCl (5 ml) was added and the organic phase was removed and washed with saturated NaHCO₃ (5 ml) and dried. Unless otherwise stated, the product was used without further purification.

### Methyl 2-(2-acetamidoethoxy)-5-chlorobenzoate (25a)

Following General Method C, amine salt 24a (1.0 g, 3.8 mmol) and acetyl chloride provided product as a pale brown solid and was used without further purification; (1.0 g, quantitative)

NMR (CDCl₃) δ 7.80 (1H, d, J 2.8), 7.44 (1H, dd, J 8.8, 2.5), 7.08 (1H, br s), 6.94 (1H, d, J 8.8), 4.14 (2H, t, J 5.1), 3.91 (3H, s), 3.73-3.62 (2H, m), 2.03 (3H, s);

MS (m/e) 272 [M+H]⁺, Rₜ 0.68 min (QC Method 2).

### Methyl 2-(3-acetamidopropoxy)-5-chlorobenzoate (26a)

Following General Method C, amine salt 24b (0.5 g, 1.8 mmol) and acetyl chloride provided product as a yellow oil after purification by column chromatography (SiO₂; EtOAc:PE is 1:1→7:3); (0.27 g, 53%)

NMR (CDCl₃) δ 7.80 (1H, d, J 2.8), 7.44 (1H, dd, J 8.8, 2.5), 7.08 (1H, br s), 6.94 (1H, d, J 8.8), 4.14 (2H, t, J 5.1), 3.91 (3H, s), 3.73-3.62 (2H, m), 2.03 (3H, s);

MS (m/e) 286 [M+H]⁺, Rₜ 0.73 min (QC Method 2).

### Methyl 5-chloro-2-(2-propionamidoethoxy)benzoate (25b)

Following General Method C, amine salt 24a (1.0 g, 3.8 mmol) and propionyl chloride provided product as a pale brown solid and was used without further purification; (1.1 g, quantitative)

NMR (CDCl₃) δ 7.80 (1H, d, J 2.8), 7.44 (1H, dd, J 8.8, 2.8), 6.97 (1H, br s), 6.93 (1H, d, J 8.8), 4.15-4.11 (2H, m), 3.91 (3H, s), 3.73-3.66 (2H, m), 2.27 (2H, q, J 7.6), 1.17 (3H, t, J 7.6);

MS (m/e) 286 [M+H]⁺, Rₜ 0.73 min (QC Method 2)

### Methyl 5-chloro-2-(3-propionamidopropoxy)benzoate (26b)

Following General Method C, amine salt 24b (1.0 g, 3.6 mmol) and propionyl chloride provided product as an orange oil after purification by column chromatography (SiO₂; MeOH:DCM 0:1→1:49); (0.68 g, 63%)

NMR (CDCl₃) δ 7.91 (1H, d, J 2.5), 7.58 (1H, br s), 7.48 (1H, dd, J 8.8, 2.8), 6.94 (1H, d, J 9.2), 4.16 (2H, t, J 5.4), 3.92 (3H, s), 3.56 (2H, dd, J 11.1, 5.4), 2.36 (2H, q, J 7.6), 2.12-2.05 (2H, m), 1.18 (3H, t, J 7.6);

MS (m/e) 300 [M+H]⁺, Rₜ 0.77 min (QC Method 2)

### Methyl 5-chloro-2-(2-(methylsulfonamido)ethoxy)benzoate (25c)

A solution of 24a (0.9 g, 3.4 mmol) in DCM (10 ml) was treated with TEA (8.9 mmol) and methylsulfonyl chloride (5.4 mmol). After 16 h, 1M HCl (5 ml) was added and the organic phase was removed and washed with saturated NaHCO₃ (5 ml) and dried (MgSO₄) to give product as a colourless oil (1.0 g, quantitative).

NMR (CDCl₃) δ 7.81 (1H, d, J 2.8), 7.44 (1H, dd, J 8.8, 2.8), 6.92 (1H, d, J 8.8), 6.06-5.95 (1H, m), 4.23 (2H, t, J 4.7), 3.90 (3H, s), 3.53 (2H, dd, J 10.1, 5.3), 3.01 (3H, s);

MS (m/e) 308 [M+H]⁺, Rₜ 0.72 min (QC Method 2)

### Methyl 5-chloro-2-(3-(methylsulfonamido)propoxy)benzoate (26c)

Synthesis analogous to 25c, using 24b.

NMR (CDCl₃) δ 7.85 (1H, d, J 2.8), 7.44 (1H, dd, J 8.8, 2.5), 6.91 (1H, d, J 8.8), 6.12 (1H, s), 4.16 (2H, t, J 5.6), 3.92 (3H, s), 3.43 (2H, dd, J 11.3, 6.0), 2.98 (3H, s), 2.18-2.09 (2H, m);

MS (m/e) 320 [M−H]⁻, Rt 0.75 min (QC Method 2)

### Methyl 5-chloro-2-(3-(ethylsulfonamido)propoxy)benzoate (26d)

Synthesis analogous to 25c, using 24b and ethylsulfonyl chloride.

NMR (CDCl₃) δ 7.89-7.87 (1H, m), 7.50-7.42 (1H, m), 6.93 (1H, d, J 8.8), 6.35 (1H, t, J 5.8), 4.16 (2H, t, J 5.5), 3.90 (3H, s), 3.47-3.40 (2H, m), 3.10 (2H, q, J 7.3), 2.20-2.11 (2H, m), 1.36 (3H, t, J 7.3);

MS (m/e) 334 [M−H]⁻, Rₜ 0.79 min (QC Method 2)

### Methyl 5-chloro-2-(2-(3-ethylureido)ethoxy)benzoate (25d)

A solution of 24a (0.9 g, 3.4 mmol) in DCM (10 ml) was treated with TEA (8.9 mmol) and methylisocyanate (5.4 mmol). After 4 hr, 1M HCl (5 ml) was added and the organic phase was removed and washed with saturated NaHCO₃ (5 ml) and dried to give the product as a white solid. This was used without further purification

(CDCl₃) δ 7.77 (1H, d, J 2.8), 7.42 (1H, dd, J 8.8, 2.8), 6.92 (1H, d, J 8.8), 5.71 (1H, br t, J 5.1), 4.61-4.48 (1H, m), 4.10 (2H, t, J 5.1), 3.89 (3H, s), 3.65-3.58 (2H, m), 3.28-3.14 (2H, m), 1.11 (3H, t, J 7.3);

MS (m/e) 301 [M+H]⁺, Rₜ 0.73 min (QC Method 2)

### General Method D: Hydrolysis of Methyl Benzoates

To a solution of the methyl ester (1.0 g, 3.5 mmol) in THF (2 mL) was added 1M NaOH and then the suspension was stirred for 4 hr, or until completion. The reaction mixture was acidified with 1M HCl until ˜pH 1 and then extracted with EtOAc (2×20 ml) and dried to provide the carboxylic acid. When required, the product was partially purified by solvating the product in DCM, washing with 1M NaOH, followed by acidification of the basic mixture and extractions with EtOAc.

### 2-(2-Acetamidoethoxy)-5-chlorobenzoic acid (27a)

Following General Method D, 25a (1.0 g, 3.8 mmol) provided the product as a white solid and was used without further purification; (1.0 g, quantitative)

NMR (CDCl₃) δ 8.04 (1H, d, J 2.6), 7.50 (1H, dd, J 8.8, 2.8), 6.99 (1H, d, J 9.2), 6.59 (1H, br s), 4.30-4.23 (2H, m), 3.78-3.69 (2H, m), 2.06 (3H, s);

MS (m/e) 258 [M+H]⁺, Rₜ 0.56 min (QC Method 2)

### 5-Chloro-2-(2-propionamidoethoxy)benzoic acid (27b)

Following General Method D, 25b (1.1 g, 3.8 mmol) provided the product as a pale brown solid and was used without further purification; (1.1 g, quantitative)

NMR (CDCl₃) δ 9.65 (1H, br s), 7.98 (1H, d, J 2.8), 7.48 (1H, dd, J 8.8, 2.8), 6.97 (1H, d, J 8.8), 6.86-6.66 (1H, m), 4.24 (2H, t, J 5.2), 3.77-3.71 (2H, m), 2.30 (2H, q, J 7.6), 1.17 (3H, t, J 7.6);

MS (m/e) 272 [M+H]⁺, Rₜ 0.61 min (QC Method 2)

### 5-Chloro-2-(2-(methylsulfonamido)ethoxy)benzoic acid (27c)

Following General Method D, 25c (1.0 g, 3.4 mmol) provided the product as a white solid and was used without further purification; (0.80 g, 80%)

NMR (CDCl₃) δ 8.03 (1H, d, J 2.5), 7.53 (1H, dd, J 8.8, 2.8), 6.99 (1H, d, J 8.8), 5.81 (1H, br s), 4.30 (2H, t, J 5.1), 3.61 (2H, t, J 5.1), 3.06 (3H, s)

### 5-Chloro-2-(2-(3-ethylureido)ethoxy)benzoic acid (27d)

Following General Method D, 25d (1.0 g, 3.4 mmol) provided the product as a white solid and was used without further purification; (0.92 g, 95%)

NMR δ 12.93 (1H, br s), 7.63 (1H, d, J 2.8), 7.54 (1H, dd, J 9.2, 2.8), 7.22 (1H, d, J 9.2), 6.05 (1H, t, J 5.5), 5.95 (1H, t, J 5.5), 4.02 (2H, t, J 5.8), 3.41-3.27 (2H, m), 3.08-2.92 (2H, m), 0.98 (3H, t, J 7.3);

MS (m/e) 287 [M+H]⁺, Rₜ 0.61 min (QC Method 2)

### 2-(3-Acetamidopropoxy)-5-chlorobenzoic acid (28a)

Following General Method D, 26a (0.27 g, 0.9 mmol) provided the product as a grey solid and was used without further purification; (0.19 g, 73%)

NMR (CDCl₃) δ 8.07 (1H, d, J 2.8), 7.52-7.44 (2H, m), 6.95 (1H, d, J 8.8), 4.20 (2H, t, J 5.7), 3.61-3.51 (2H, m), 2.15 (3H, s), 2.14-2.06 (2H, m);

MS (m/e) 272 [M+H]⁺, Rₜ 0.61 min (QC Method 2)

### 5-Chloro-2-(3-propionamidopropoxy)benzoic acid (28b)

Following General Method D, 26b (0.70 g, 2.3 mmol) provided the product as a brown solid and was used without further purification; (0.67 g, quantitative)

NMR (CDCl₃) δ 8.03 (1H, d, J 2.8), 7.48 (1H, dd, J 8.8, 2.8), 6.95 (1H, d, J 9.2), 4.23-4.06 (2H, m), 3.61-3.46 (2H, m), 2.45-2.28 (2H, m), 2.16-1.99 (2H, m), 1.17 (3H, t, J 7.0);

MS (m/e) 286 [M+H]⁺, Rₜ 0.65 min (QC Method 2)

### 5-Chloro-2-(3-(methylsulfonamido)propoxy)benzoic acid (28c)

Following General Method D, 26c (0.075 g, 0.2 mmol) provided the product as a colourless oil and was used without further purification; (0.065 g, 87%)

NMR (CDCl₃) δ 8.43 (1H, br s), 7.85 (1H, d, J 2.8), 7.35 (1H, dd, J 8.8, 2.5), 6.86 (1H, d, J 9.2), 6.00 (1H, br s), 4.14 (2H, t, J 5.7), 3.34-3.20 (2H, m), 2.88 (3H, s), 2.11-1.96 (2H, m);

MS (m/e) 306 [M−H]⁻, Rₜ 0.63 min (QC Method 2)

### 5-Chloro-2-(3-(ethylsulfonamido)propoxy)benzoic acid (28d)

Following General Method D, 26d (0.51 g, 1.5 mmol) provided the title compound as a grey solid and was used without further purification; (0.49 g, quantitative)

NMR (CDCl₃) δ 7.99 (1H, d, J 2.8), 7.45 (2H, dd, J 9.2, 2.8), 6.93 (1H, d, J 8.8), 6.19 (1H, br s), 6.18 (1H, t, J 5.8), 4.23 (2H, t, J 5.8), 3.37 (2H, dd, J 11.4, 5.8), 3.07 (2H, q, J 7.3), 2.19-2.10 (2H, m), 1.35 (3H, t, J 7.3);

MS (m/e) 320 [M−H]⁻, Rₜ 0.67 min (QC Method 2)

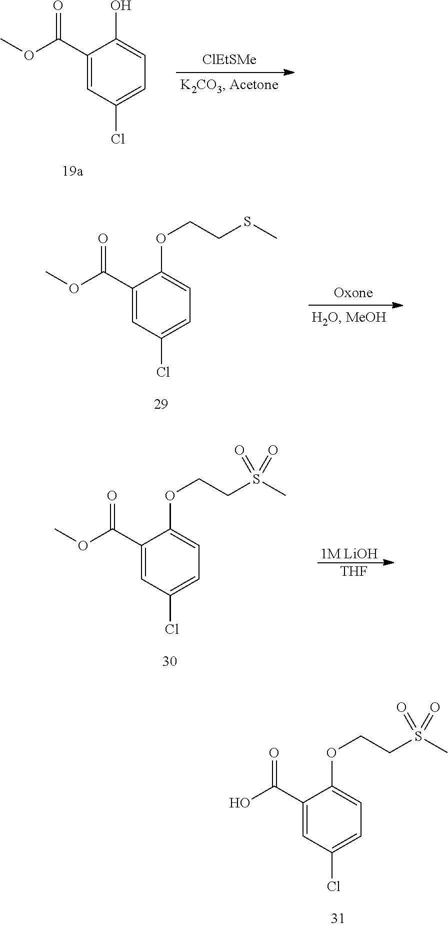

### Methyl 5-chloro-2-(2-(methylthio)ethoxy)benzoate (29)

A suspension of 19a (1.70 g, 9.1 mmol), 2-chloroethyl methyl sulfide (1.10 g, 11 mmol) and K₂CO₃ (2.5 g, 18 mmol) in acetone (50 ml) was heated at reflux overnight. The reaction was cooled to RT, filtered and concentrated. Column chromatography (SiO₂; 1:1 PE:ether) gave the title compound; (1.2 g, 4.6 mmol)

NMR (CDCl₃) δ 7.76 (1H, d, J 2.8), 7.40 (1H, dd, J 8.8, 2.8), 6.92 (1H, d, J 8.8), 4.20 (2H, t, J 6.9), 3.88 (3H, s), 2.92 (2H, t, J 6.9), 2.22 (3H, s)

### Methyl 5-chloro-2-(2-(methylsulfonyl)ethoxy)benzoate (30)

A suspension of 29 (1.2 g, 4.6 mmol) in 3:1 MeOH:water (60 ml) was treated portionwise with Oxone® (10 g, 16 mmol) and stirred overnight. The MeOH was removed under vacuum and the aqueous washed with DCM. The organics were dried and concentrated to give the title compound as a white solid; (1.11 g, 3.8 mmol)

NMR (CDCl₃) δ 7.82 (1H, d, J 2.8), 7.45 (1H, dd, J 8.8, 2.8), 6.96 (1H, d, J 8.8), 4.47 (2H, t, J 5.2), 3.87 (3H, s), 3.47 (2H, t, J 5.2), 3.16 (3H, s)

### 5-Chloro-2-(2-(methylsulfonyl)ethoxy)benzoic acid (31)

A solution of 30 (1.11 g, 3.8 mmol) in THF (50 ml) was treated with 1M LiOH (25 ml) and stirred at RT for 24 hrs. The mixture was extracted with ether, the organics discarded, and the aqueous acidified. This was washed with DCM, the organics dried and concentrated to give the title compound as a white solid; (1.02 g, 3.7 mmol)

NMR δ 7.65 (1H, d, J 2.8), 7.57 (1H, dd, J 8.8, 2.8), 7.22 (1H, d, J 8.8), 4.38 (2H, t, J 5.3), 3.61 (2H, t, J 5.3), 3.12 (3H, s);

MS (m/e) No ion observed, Rₜ 1.18 min (QC Method 1)

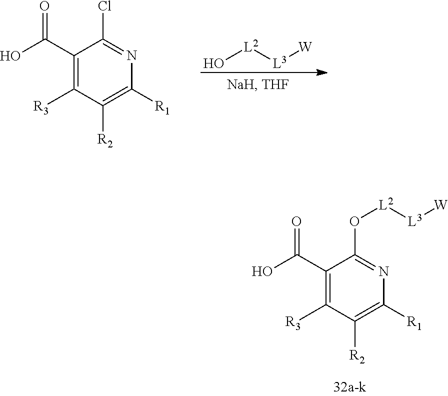

### General Method E: Preparation of 2-alkoxypyridines

### Exemplified by: 5-Fluoro-2-(2-methoxyethoxy)nicotinic acid (32a)

A solution of 2-chloro-5-fluoronicotinic acid (18.7 g, 0.11 mol) and 2-methoxyethanol (25.4 g, 0.33 mol) in THF (1.5 L) was cooled to 0° C. and treated portionwise with NaH (60% dispersion in mineral oil, 17.2 g, 0.43 mol) and stirred under N₂ for 2 days. The reaction was diluted with 1M NaOH and extracted with ether, which was discarded. The aqueous was acidified with conc. HCl and extracted with DCM. The organics were dried and is concentrated to give the title compound as an off-white solid; (19.93, 92.7 mmol)

Further purification, where necessary, was by column chromatography (SiO₂; 1:99 MeOH:DCM)

NMR δ 8.34 (1H, d, J 3.2), 8.01 (1H, dd, J 8.2, 3.2), 4.44 (2H, m), 3.67 (2H, m), 3.31 (3H, s);

MS (m/e) 216 [M+H]⁺, Rₜ 1.14 min (QC Method 1)

### 2-(2-Methoxyethoxy)nicotinic acid (32b)

Prepared according to General Method E, using 2-chloronicotinic acid

NMR (CDCl₃) δ 8.47 (1H, dd, J 7.6, 2.2), 8.38 (1H, dd, J 5.0, 1.9), 7.15 (1H, dd, J 7.6, 4.7), 4.75 (2H, m), 3.83 (2H, m), 3.48 (3H, s);

MS (m/e) 198 [M+H]⁺, Rₜ 0.56 min (QC Method 2)

### 5-Chloro-2-(2-methoxyethoxy)nicotinic acid (32c)

Prepared according to general method E, using 2,5-dichloronicotinic acid

NMR (CDCl₃) δ 8.37 (1H, dd, J 2.8, 1.8), 8.11 (1H, dd, J 2.8, 1.8), 4.43 (2H, m), 3.64 (2H, m), 3.28 (3H, s);

MS (m/e) 230 [M−H]⁻, Rₜ 1.24 min (QC Method 1)

### 5-Bromo-2-(2-methoxyethoxy)nicotinic acid (32d)

Prepared according to General Method E, using 5-bromonicotinic acid

NMR δ 8.42 (1H, d, J 2.5), 8.19 (1H, d, J 2.8), 4.43 (2H, m), 3.65 (2H, m), 3.29 (3H, s);

MS (m/e) 178 [M+H]⁺, Rₜ 0.73 min (QC Method 2)

### 2-(2-Ethoxyethoxy)-5-fluoronicotinic acid (32e)

Prepared according to General Method E, using 2-ethoxyethanol

NMR δ 13.23 (1H, br), 8.32 (1H, d, J 3.2), 7.99 (1H, dd, J 8.2, 3.2), 4.40 (2H, m), 3.68 (2H, m), 3.49 (2H, q, J 7.0), 1.08 (3H, t, J 7.0);

MS (m/e) 230 [M+H]⁺, Rₜ 1.23 min (QC Method 1)

### (S)-5-Fluoro-2-(2-methoxypropoxy)nicotinic acid (32f)

Prepared according to general method E, using (S)-2-methoxypropanol

NMR (CDCl₃) δ 8.19-8.25 (2H, m), 4.67 (1H, dd, J 11.1, 3.2), 4.36 (1H, dd, J 11.1, 7.3), 3.81 (1H, m), 3.45 (3H, s), 1.30 (3H, d, J 6.3)

### 5-Fluoro-2-(2-isopropoxyethoxy)nicotinic acid (32 g)

Prepared according to general Method E, using 2-isopropoxyethanol

NMR (CDCl₃) δ 8.20-8.25 (2H, m), 4.69 (2H, m), 3.84 (2H, m), 3.71 (1H, m), 1.23 (6H, d, J 6.0)

### 5-Fluoro-2-(2-(2-methoxyethoxy)ethoxy)nicotinic acid (32h)

Prepared according to General Method E, using 2-(2-methoxyethoxy)ethanol

NMR (CDCl₃) δ 8.19-8.25 (2H, m), 4.73 (2H, m), 3.93 (2H, m), 3.74 (2H, m), 3.62 (2H, m), 3.42 (3H, s);

MS (m/e) 260 [M+H]⁺, Rₜ 0.66 min (QC Method 2)

### 5-Fluoro-2-(3-methoxypropoxy)nicotinic acid (32i)

Prepared according to General Method E, using 3-methoxypropanol

NMR (CDCl₃) δ 8.19-8.25 (2H, m), 4.66 (2H, t, J=5.7 Hz), 3.66 (2H, t, J=5.7 Hz), 3.40 (3H, s), 2.15 (2H, m)

### 2-(3-Ethoxypropoxy)-5-fluoronicotinic acid (32j)

Prepared according to General Method E, using 3-ethoxypropanol

NMR (CDCl₃) δ 8.20-8.26 (2H, m), 4.68 (2H, t, J 6.0), 3.67 (2H, m), 3.55 (2H, q, J 7.0), 2.16 (2H, m), 1.24 (3H, t, J 7.0)

### 2-(2-Methoxyethoxy)-4-methylnicotinic acid (32k)

Prepared according to General Method E, using 2-chloro-4-methylnicotinic acid

NMR (CDCl₃) δ 8.36 (1H, d, J 7.9), 6.99 (1H, d, J 7.9), 4.74 (2H, m), 3.81 (2H, m), 3.46 (3H, s), 2.54 (3H, s)

### EXAMPLES

### General Method F: Examples from PMB-benzodiazepine amine

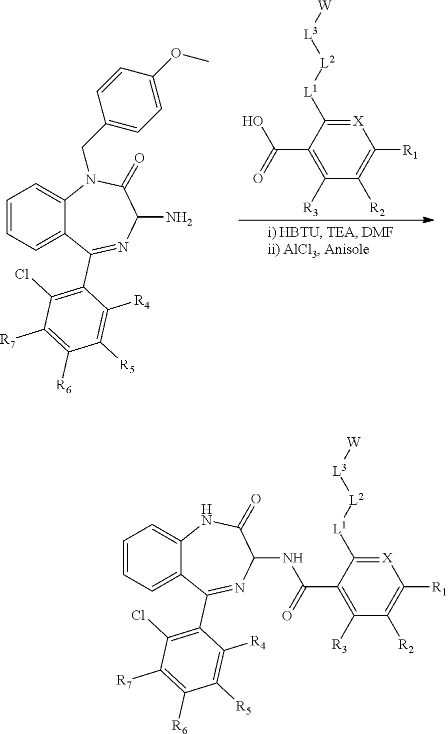

After 30 min, a stirred solution of carboxylic acid (0.30 mmol), HBTU (0.36 mmol) and TEA (0.72 mmol) in DMF (3 ml) was treated with a solution PMB-benzodiazepine amine (0.24 mMol) in DMF (3 ml) and stirred overnight.

The reaction mixture was concentrated, the residue taken into DCM and washed with saturated NaHCO₃ solution and 2M HCl. The organics were dried and concentrated.

The resulting residue was taken into anisole (3 ml), treated with aluminium trichloride (7.5 mmol) and stirred overnight at RT. This was diluted with EtOAc, basified with saturated Na₂CO₃ and the resulting slurry filtered through Celite®. The organics were separated, dried and concentrated. Purification by column chromatography gave the final compound.

### General Method G: Examples from Benzodiazepine Amine

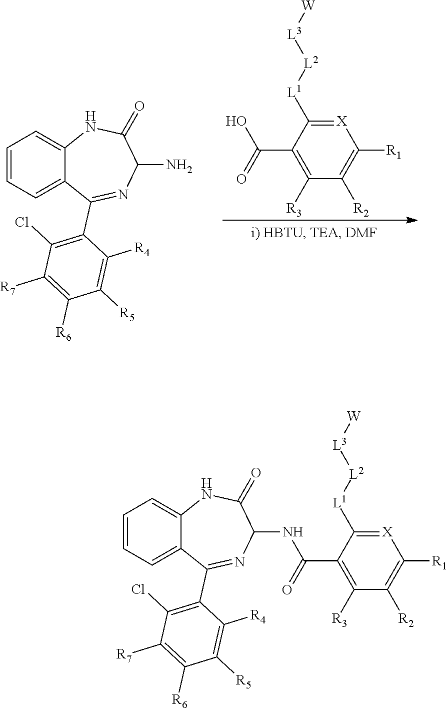

is After 30 min, a solution of carboxylic acid (0.3 mmol), HBTU (0.36 mmol) and TEA (0.72 mmol) in DMF (3 ml) was treated with benzodiazepine amine (0.26 mmol) and the reaction stirred at RT overnight. After concentration, the reaction residue was taken into DCM, washed with saturated Na₂CO₃ solution and 2M HCl. The organics were dried and concentrated. Purification by column chromatography gave the final compound.

### Example 1

### 5-chloro-2-(3-methoxypropoxy)-N-(2-oxo-5-(2,4,6-trichlorophenyl)-2,3-dihydro-1H-benzo[e][1,4]diazepin-3-yl)benzamide

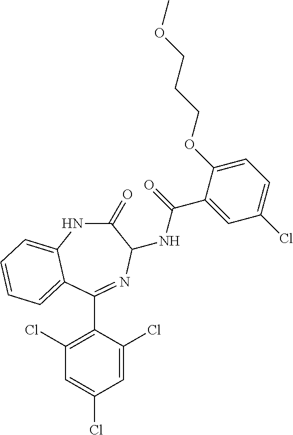

Prepared according to General Method G, using 9 and 18a. Purification by preparative HPLC

NMR δ 11.27 (1H, s), 9.57 (1H, d, J 7.5), 7.89 (1H, d, J 1.8), 7.87 (1H, d, J 3), 7.72 (1H, d, J 1.8), 7.55-7.63 (2H, m), 7.18-7.31 (4H, m), 5.55 (1H, d, J 7.5), 4.25 (2H, m), 3.52 (2H, t, J 6.2), 3.33 (3H, s), 2.11 (2H, m);

MS (m/e) 582 [M+H]⁺, Rₜ 1.15 min (QC Method 2)

### Example 2

### 5-Chloro-2-((S)-2-methoxypropoxy)-N-(2-oxo-5-(2,4,6-trichlorophenyl)-2,3-dihydro-1H-benzo[e][1,4]diazepin-3-yl)benzamide

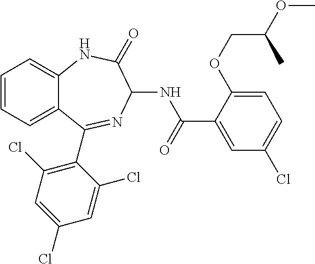

Prepared according to General Method F, using 8b and 18b. Purification by column chromatography (SiO₂; DCM→200:8:1 DCM:EtOH:NH₃)

NMR δ 11.23 (1H, br), 9.55 (0.5H, d, J 7.6), 9.47 (0.5H, d, J 7.6), 7.92 (1H, d, J 2.2), 7.86 (1H, m), 7.74 (1H, d, J 1.9), 7.56-7.66 (2H, m), 7.17-7.33 (4H, m), 5.57 (0.5H, d, J 7.6), 5.55 (0.5H, d, J 7.6), 4.16 (2H, m), 3.82 (1H, m), 3.19 (1.5H, s), 3.17 (1.5H, s), 1.17 (3H, m);

MS (m/e) 582 [M+H]⁺, Rₜ 3.62 min (QC Method 4)

### Example 3

### 5-Chloro-2-(2-ethoxyethoxy)-N-(2-oxo-5-(2,4,6-trichlorophenyl)-2,3-dihydro-1H-benzo[e][1,4]diazepin-3-yl)benzamide

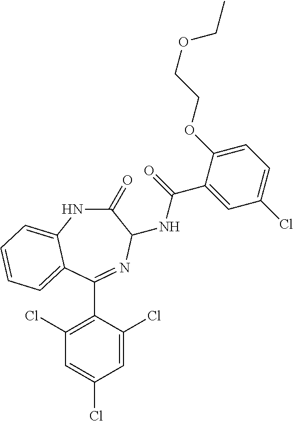

Prepared according to General Method F, using 8b and 18c. Purification by column chromatography (SiO₂; PE→EtOAc)

NMR δ 11.25 (1H, s), 9.56 (1H, d, J 7.6), 7.93 (1H, d, J 1.9), 7.91 (1H, d, J 2.8), 7.76 (1H, d, J 1.9), 7.59-7.66 (2H, m), 7.18-7.35 (4H, m), 5.59 (1H, d, J 7.6), 3.82 (2H, m), 3.40 (2H, m), 1.10 (2H, t, J 7.0), 0.93 (3H, t, J 7.0);

MS (m/e) 580 [M+H]⁺, Rₜ 3.59 min (QC Method 4)

### Example 4

### 5-Chloro-2-(1-methoxypropan-2-yloxy)-N-(2-oxo-5-(2,4,6-trichlorophenyl)-2,3-dihydro-1H-benzo[e][1,4]diazepin-3-yl)benzamide

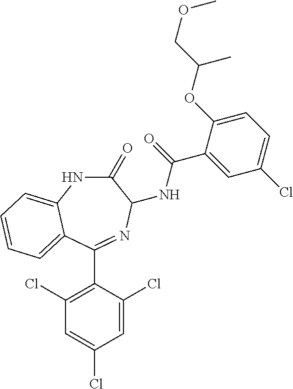

Prepared according to General Method F, using 8b and 18d. Purification by column chromatography (SiO₂; PE→EtOAc)

NMR δ 10.71 (1H, br), 9.62 (0.5H, d, J 7.0), 9.58 (0.5H, d, J 7.0), 7.84-7.90 (2H, m), 7.52-7.70 (3H, m), 7.14-7.37 (4H,m), 5.56 (0.5H, d, J 7.3), 5.54 (0.5H, d, J 7.3), 4.88 (1H, m), 3.72 (0.5H, m), 3.68 (0.5H, m), 3.59 (0.5H, m), 3.54 (0.5H, m), 3.23 (1.5H, s), 3.20 (1.5H, s), 1.36 (1.5H, d, J 2.5), 1.34 (1.5H, d, J 2.5);

MS (m/e) 580 [M+H]⁺, Rₜ 3.59, 3.62 min (QC Method 4)

### Example 5

### 5-Chloro-2-(2-(2-methoxyethoxy)ethoxy)-N-(2-oxo-5-(2,4,6-trichlorophenyl)-2,3-dihydro-1H-benzo[e][1,4]diazepin-3-yl)benzamide

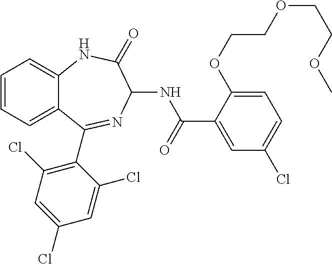

Prepared according to General Method F, using 8b and 18e. Purification by column chromatography (SiO₂; DCM→200:8:1 DCM:EtOH:NH₃)

NMR δ 11.25 (1H, s), 9.56 (1H, d, J 7.6), 7.92 (1H, d, J 2.2), 7.89 (1H, d, J 2.8), 7.75 (1H, d, J 2.2), 7.54-7.64 (2H, m), 7.17-7.33 (4H, m), 5.56 (1H, d, J 7.6), 4.33 (2H, m), 3.84 (2H, m), 3.46 (2H, m), 3.22 (2H, m), 3.03 (3H, s);

MS (m/e) 610 [M+H]⁺, Rₜ 3.41 min (QC Method 4)

### Example 6

### 5-Chloro-2-(2-isobutoxyethoxy)-N-(2-oxo-5-(2,4,6-trichlorophenyl)-2,3-dihydro-1H-benzo[e][1,4]diazepin-3-yl)benzamide

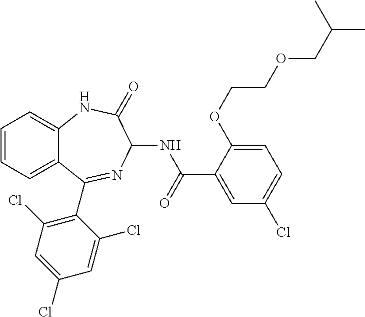

Prepared according to General Method F, using 8b and 18f. Purification by preparative HPLC

NMR δ 11.21 (1H, br), 9.53 (1H, d, J 7.6), 7.91 (2H, m), 7.74 (1H, d, J 2.2), 7.63 (2H, m), 7.31-7.19 (4H, m), 5.58 (1H, d, J 7.6), 4.36 (2H, m), 3.84 (2H, m), 3.15 (2H, dd, J 7.0, 2.2), 1.69 (1H, m), 0.75 (6H, d, J 6.6);

MS (m/e) 608 [M+H]⁺, Rₜ 3.93 min (QC Method 4)

### Example 7

### 5-Chloro-N-(2-oxo-5-(2,4,6-trichlorophenyl)-2,3-dihydro-1H-benzo[e][1,4]diazepin-3-yl)-2-(2-(pentyloxy)ethoxy)benzamide

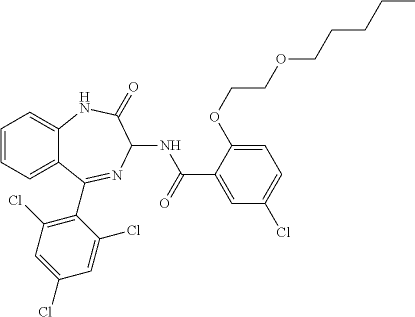

Prepared according to General Method F, using 8b and 18g. Purification by preparative HPLC

NMR δ 11.21 (1H, br), 9.56 (1H, d, J 7.6), 7.91 (2H, m), 7.75 (1H, d, J 1.9), 7.68-7.59 (2H, m), 7.36-7.19 (4H, m), 5.60 (1H, d, J 7.6), 4.36 (2H,m), 3.86 (2H, m), 3.72 (2H, m), 3.50 (2H, m), 3.31 (2H, m), 3.20 (2H, m), 1.00 (3H, t, J 7.3);

MS (m/e) 623 [M+H]⁺, Rₜ 3.55 min (QC Method 4)

### Example 8

### 5-Chloro-2-(2-(2-ethoxyethoxy)ethoxy)-N-(2-oxo-5-(2,4,6-trichlorophenyl)-2,3-dihydro-1H-benzo[e][1,4]diazepin-3-yl)benzamide

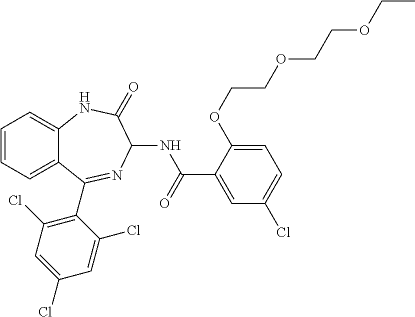

Prepared according to General Method F, using 8b and 18h. Purification by preparative HPLC

NMR δ 11.20 (1H, br), 9.55 (1H, d J 7.3), 7.91 (2H, m), 7.73 (1H, d, J 1.9), 7.63 (2H, m), 7.20-7.30 (4H, m), 5.58 (1H, d, J 7.3), 4.36 (2H, m), 3.82 (2H, m), 1.34 (2H, m), 1.12-1.04 (4H, m), 0.75 (3H, m);

MS (m/e) 622 [M+H]⁺, Rₜ 4.06 min (QC Method 4)

### Example 9

### 5-Chloro-N-(2-oxo-5-(2,4,6-trichlorophenyl)-2,3-dihydro-1H-benzo[e][1,4]diazepin-3-yl)-2-((tetrahydrofuran-2-yl)methoxy)benzamide

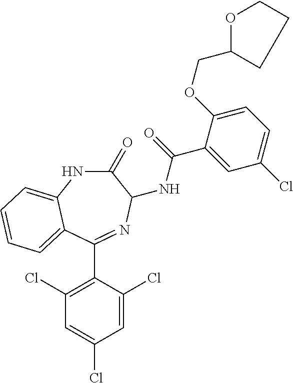

Prepared according to General Method F, using 8b and 18i. Purification by column chromatography (SiO₂; PE→EtOAc)

NMR δ 11.11 (1H, br), 9.40 (0.5H, d, J 7.0), 9.38 (0.5H, d, J 7.0), 7.83-7.88 (2H, m), 7.69 (1H, d, J 1.9), 7.53-7.66 (2H, m), 7.15-7.34 (4H, m), 5.57 (0.5H, d, J 7.5), 5.56 (0.5H, d, J 7.5), 4.15-4.29 (3H, m), 3.72 (1H, m), 3.56 (1H, m), 2.01 (1H, m), 1.66-1.85 (3H, m);

MS (m/e) 592 [M+H]⁺, Rₜ 3.58 min (QC Method 4)

### Example 10

### 2-(2-Butoxyethoxy)-5-chloro-N-(2-oxo-5-(2,4,6-trichlorophenyl)-2,3-dihydro-1H-benzo[e][1,4]diazepin-3-yl)benzamide

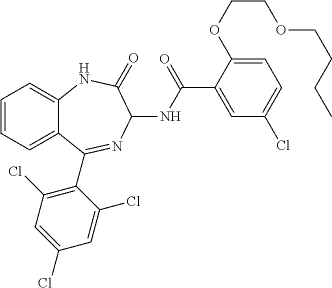

Prepared according to General Method F, using 8b and 18j. Purification by preparative HPLC

NMR δ 11.24 (1H, br), 9.55 (1H, d, J 7.6), 7.91 (1H, d, J 1.9), 7.88 (1H, d, J 2.8), 7.73 (1H, d, J 1.9), 7.57-7.63 (2H, m),7.14-7.32 (4H, m), 5.55 (1H, d, J 7.6), 4.32 (2H, m), 3.81 (2H, m), 1.28 (2H, m), 1.02-1.16 (4H, m);

MS (m/e) 608 [M+H]⁺, Rₜ 3.76 min (QC Method 3)

### Example 11

### 5-Chloro-N-(2-oxo-5-(2,4,6-trichlorophenyl)-2,3-dihydro-1H-benzo[e][1,4]diazepin-3-yl)-2-(2-phenoxyethoxy)benzamide

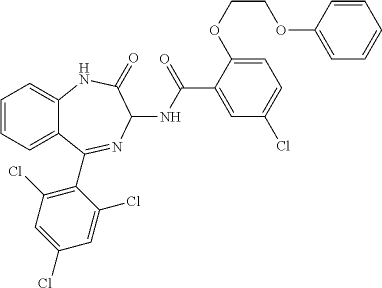

Prepared according to General Method F, using 8b and 18k. Purification by preparative HPLC

NMR δ 11.20 (1H, br), 9.60 (1H, d, J 7.6), 7.88 (1H, d, J 2.8), 7.86 (1H, d, J 1.9), 7.71 (1H, d, J 1.9), 7.56-7.65 (2H, m), 7.10-7.30 (6H, m), 6.82-6.91 (3H, m), 5.52 (1, d, J 7.6), 4.58 (2H, m), 4.43 (2H, m);

MS (m/e) 630 [M+H]⁺, Rₜ 1.09 min (QC Method 2)

### Example 12

### 5-Chloro-2-(3-(dimethylamino)propoxy)-N-(2-oxo-5-(2,4,6-trichlorophenyl)-2,3-dihydro-1H-benzo[e][1,4]diazepin-3-yl)benzamide

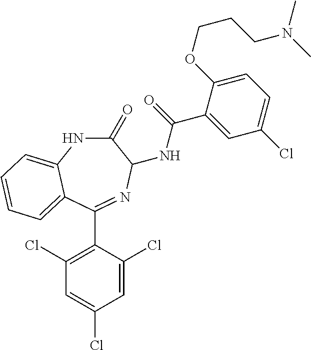

Prepared according to General Method F, using 8b and 5-chloro-2-(3-(dimethylamino)propoxy)benzoic acid. Purification by preparative HPLC

NMR δ 11.27 (1H, Br), 9.52 (1H, d, J 7.3), 7.90 (1H, d, J 1.9), 7.86 (1H, d, J 2.5), 7.72 (1H, d, J 1.9), 7.66-7.55 (2H, m), 7.33-7.15 (4H, m), 5.55 (1H, d, J 7.3), 4.23 (2H, m), 2.57 (2H, m), 2.16 (6H, s), 2.05 (2H, m);

MS (m/e) 593 [M+H]⁺, Rₜ 2.51 min (QC Method 4)

### Example 13

### 5-Chloro-2-(2-(2-(dimethylamino)ethoxy)ethoxy)-N-(2-oxo-5-(2,4,6-trichlorophenyl)-2,3-dihydro-1H-benzo[e][1,4]diazepin-3-yl)benzamide

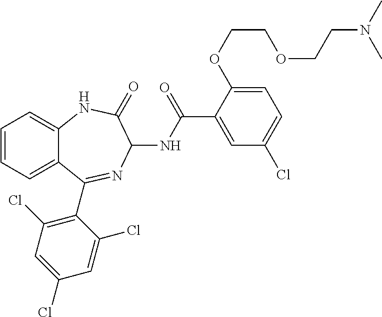

Prepared according to General Method F, using 8b and 18l. Purification by preparative HPLC

NMR δ 11.22 (1H, br), 9.58 (1H, d, J 7.6), 7.89-7.94 (2H, m), 7.74 (1H, d, J 1.9), 7.58-7.69 (2H, m), 7.20-7.36 (4H, m), 5.60 (1H, d, J 7.6), 4.36 (2H, m), 3.89 (2H, m), 3.51 (2H, m), 2.38 (2H, m), 2.09 (6H, s);

MS (m/e) 623 [M+H]⁺, Rₜ 0.79 min (QC Method 2)

### Example 14

### 5-Chloro-2-(2-methoxyethoxy)-N-(2-oxo-5-(2,4,6-trichlorophenyl)-2,3-dihydro-1H-benzo[e][1,4]diazepin-3-yl)benzamide

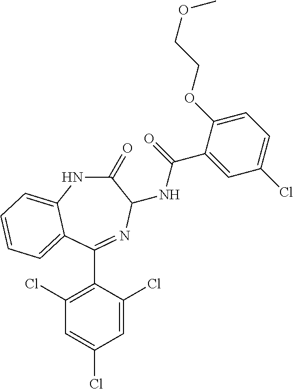

Prepared according to General Method F, using 8b and 21a. Purification by column chromatography (SiO₂; DCM→200:8:1 DCM:EtOH:NH₃)

NMR δ 11.26 (1H, s), 9.61 (1H, d, J 7.3), 7.93 (1H, d, J 1.9), 7.90 (1H, d, J 2.8), 7.75 (1H, d, J 1.9), 7.58-7.69 (2H, m), 7.18-7.36 (4H, m), 5.58 (1H, d, J 7.3), 4.36 (2H, m), 3.81 (2H, m), 3.23 (3H, s);

MS (m/e) 568 [M+H]⁺, Rₜ 3.31 min (QC Method 3)

### Example 15

### 2-(2-Methoxyethoxy)-N-(2-oxo-5-(2,4,6-trichlorophenyl)-2,3-dihydro-1H-benzo[e][1,4]diazepin-3-yl)benzamide

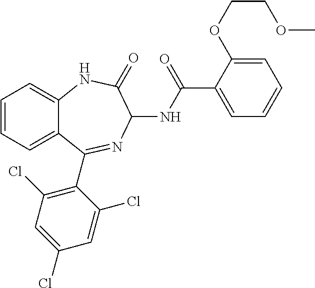

Prepared according to General Method G, using 9 and 21b. Purification by preparative HPLC

NMR δ 11.21 (1H, s), 9.59 (1H, d, J 7.3), 7.96 (1H, dd, J 7.6, 1.6), 7.91 (1H, d, J 1.9), 7.74 (1H, d, J 1.9), 7.49-7.66 (2H, m), 7.30 (1H, d, J 8.2), 7.15-7.25 (3H, m), 7.08 (1H, m), 5.57 (1H, d, J 7.6), 4.32 (2H, m), 3.80 (2H, m), 3.21 (3H, s);

MS (m/e) 534 [M+H]⁺, Rₜ 1.03 min (QC Method 2)

### Example 16

### 5-Fluoro-2-(2-methoxyethoxy)-N-(2-oxo-5-(2,4,6-trichlorophenyl)-2,3-dihydro-1H-benzo[e][1,4]diazepin-3-yl)benzamide

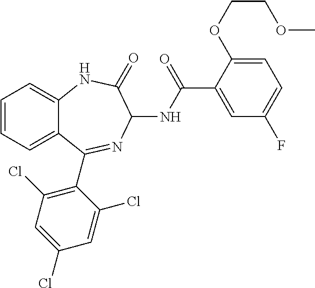

Prepared according to General Method G, using 9 and 21c. Purification by preparative HPLC

NMR δ 11.26 (1H, br, s), 9.68 (1H, d, J 7.3), 7.93 (1H, d, J 1.9), 7.76 (1H, d, J 1.9), 7.61-7.72 (2H, m), 7.44 (1H, m), 7.18-7.36 (4H, m), 5.58 (1H, d, J 7.6), 4.34 (2H, m), 3.81 (2H, m), 3.23 (3H, s);

MS (m/e) 550 [M+H]⁺, Rₜ 1.07 min (QC Method 2)

### Example 17

### 2-(2-Methoxyethoxy)-5-methyl-N-(2-oxo-5-(2,4,6-trichlorophenyl)-2,3-dihydro-1H-benzo[e][1,4]diazepin-3-yl)benzamide

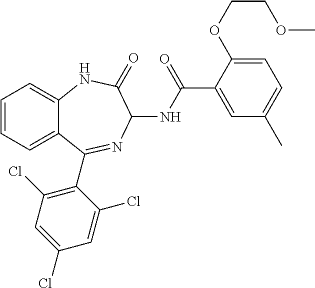

Prepared according to General Method G, using 9 and 21d. Purification by column chromatography (SiO₂; DCM→200:8:1 DCM:EtOH:NH₃)

NMR δ 11.21 (1H, s), 9.60 (1H, d, J 7.3), 7.91 (1H, d, J 1.9), 7.77 (1H, d, J 2.2), 7.73 (1H, d, J 1.9), 7.62 (1H, m), 7.09-7.36 (5H, m), 5.56 (1H, d, J 7.6), 4.28 (2H, m), 3.79 (1H, m), 3.20 (3H, s), 2.27 (3H, s);

MS (m/e) 546 [M+H]⁺, Rₜ 3.19 min (QC Method 3)

### Example 18

### 5-Acetyl-2-(2-methoxyethoxy)-N-(2-oxo-5-(2,4,6-trichlorophenyl)-2,3-dihydro-1H-benzo[e][1,4]diazepin-3-yl)benzamide

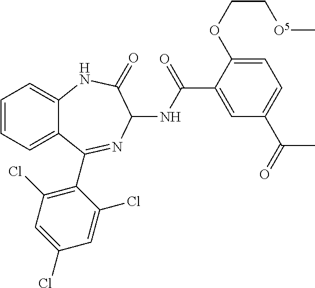

Prepared according to General Method G, using 9 and 21e. Purification by preparative HPLC

NMR δ 11.23 (1H, br), 9.56 (1H, d, J 7.3), 8.54 (1H, d, J 2.2), 8.15 (1H, dd, J 8.5, 2.2), 7.92 (1H, d, J 1.9), 7.75 (1H, d, J 2.2), 7.65 (1H, m), 7.21-7.40 (5H, m), 5.61 (1H, d, J 7.3), 4.45 (2H, m), 3.85 (2H, m), 3.25 (3H, s), 2.58 (3H, s);

MS (m/e) 574 [M+H]⁺, Rₜ 2.90 min (QC Method 3)

### Example 19

### 2-(2-Methoxyethoxy)-N-(2-oxo-5-(2,4,6-trichlorophenyl)-2,3-dihydro-1H-benzo[e][1,4]diazepin-3-yl)-5-(trifluoromethoxy)benzamide

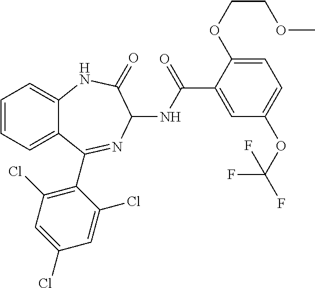

Prepared according to General Method G, using 9 and 21f. Purification by preparative HPLC

NMR δ 11.24 (1H, br), 9.63 (1H, d, J 7.3), 7.91 (1H, d, J 1.9), 7.83 (1H, d, J 3.2), 7.74 (1H, d, J 2.2), 7.55-7.66 (2H, m), 7.16-7.39 (4H, m), 5.56 (1H, d, J 7.3), 4.37 (2H, m), 3.80 (2H, m), 3.21 (3H, s);

MS (m/e) 616 [M+H]⁺, Rₜ 1.15 min (QC Method 2)

### Example 20

### 5-Bromo-2-(2-methoxyethoxy)-N-(2-oxo-5-(2,4,6-trichlorophenyl)-2,3-dihydro-1H-benzo[e][1,4]diazepin-3-yl)benzamide

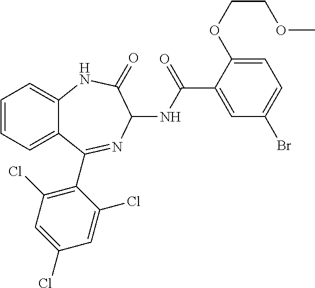

Prepared according to General Method G, using 9 and 21g. Purification by preparative HPLC

NMR δ 11.23 (1H, br), 9.58 (1H, d, J 7.3), 8.00 (1H, d, J 2.5), 7.91 (1H, d, J 1.9), 7.73 (1H, d, J 1.9), 7.57-7.70 (2H, m), 7.17-7.33 (4H, m), 5.55 (1H, d, J 7.5), 4.33 (2H, m), 3.79 (2H, m), 3.20 (3H, s);

MS (m/e) 612 [M+H]⁺, Rₜ 1.13 min (QC Method 2)

### Example 21

### 2-(2-Acetamidoethoxy)-5-chloro-N-(2-oxo-5-(2,4,6-trichlorophenyl)-2,3-dihydro-1H-benzo[e][1,4]diazepin-3-yl)benzamide

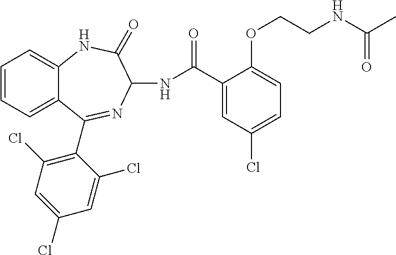

Prepared according to General Method F, using 8b and 27a. Purification by reverse phase chromatography (C18, MeCN:H₂O 7:13→4:1 over 30 min)

NMR δ 11.31 (1H, s), 9.58 (1H, d, J 7.6), 7.93-7.89 (2H, m), 7.86 (1H, d, J 2.8), 7.74 (1H, d, J 1.9), 7.70-7.55 (2H, m), 7.39-7.17 (4H, m), 5.61 (1H, d, J 7.6), 4.26-4.19 (2H, m), 3.65-3.51 (2H, m), 1.77 (3H, s);

MS (m/e) 595 [M+H]⁺, Rₜ 0.93 min (QC Method 2)

### Example 22

### 5-Chloro-N-(2-oxo-5-(2,4,6-trichlorophenyl)-2,3-dihydro-1H-benzo[e][1,4]diazepin-3-yl)-2-(2-propionamidoethoxy)benzamide

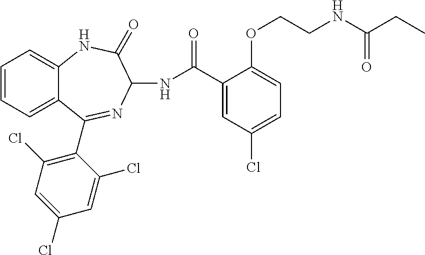

Prepared according to General Method F, using 8b and 27b. Purification by reverse phase chromatography (C18, MeCN:H₂O 7:13→4:1 over 30 min)

NMR δ 11.33 (1H, br s), 9.59 (1H, d, J 7.3), 7.91 (1H, d, J 1.9), 7.86 (1H, d, J 2.8), 7.84-7.76 (1H, m), 7.74 (1H, d, J 2.2), 7.71-7.61 (1H, m), 7.60 (1H, dd, J 8.8, 2.8), 7.39-7.17 (4H, m), 5.61 (1H, d, J 7.6), 4.24 (2H, t, J 5.4), 3.59 (2H, t, J 5.4), 2.03 (2H, q, J 7.6), 0.92 (3H, t, J 7.6);

MS (m/e) 609 [M+H]⁺, Rₜ 0.96 min (QC Method 2)

### Example 23

### 5-Chloro-2-(2-(methylsulfonamido)ethoxy)-N-(2-oxo-5-(2,4,6-trichlorophenyl)-2,3-dihydro-1H-benzo[e][1,4]diazepin-3-yl)benzamide

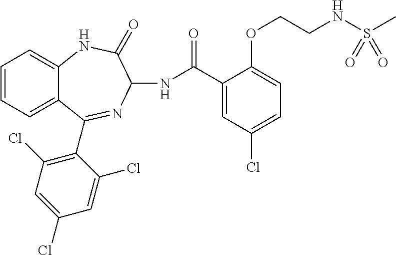

Prepared according to General Method F, using 8b and 27c. Purification by reverse phase chromatography (C18, MeCN:H₂O 7:13→4:1 over 30 min)

NMR δ 11.32 (1H, br s), 9.58 (1H, d, J 7.3), 7.91 (1H, d, J 1.9), 7.85 (1H, d, J 2.8), 7.74 (1H, d, J 1.9), 7.69-7.57 (2H, m), 7.37-7.15 (5H, m), 5.58 (1H, d, J 7.6), 4.36-4.24 (2H, m), 3.60-3.50 (2H, m), 2.94 (3H, s);

MS (m/e) 631 [M+H]⁺, Rₜ 0.95 min (QC Method 2)

### Example 24

### 5-Chloro-2-(2-(3-ethylureido)ethoxy)-N-(2-oxo-5-(2,4,6-trichlorophenyl)-2,3-dihydro-1H-benzo[e][1,4]diazepin-3-yl)benzamide

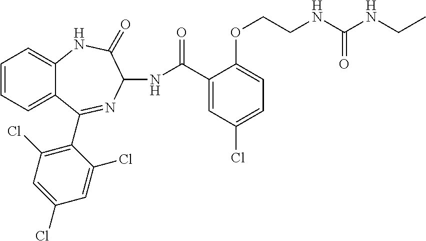

Prepared according to General Method F, using 8b and 27d. Purification by reverse phase chromatography (C18, MeCN:H₂O 7:13→4:1 over 30 min)

NMR δ 11.34 (1H, s), 9.66 (1H, d, J 7.3), 7.91 (1H, d, J 1.9), 7.88 (1H, d, J 2.8), 7.73 (1H, d, J 1.9), 7.70-7.62 (1H, m), 7.59 (1H, dd, J 8.8, 2.8), 7.38-7.18 (4H, m), 6.04 (1H, t, J 6.0), 5.96 (1H, t, J 5.5), 5.58 (1H, d, J 7.0), 4.28-4.11 (2H, m), 3.64-3.53 (2H, m), 3.05-2.92 (2H, m), 0.96 (3H, t, J 7.1);

MS (m/e) 624 [M+H]⁺, Rₜ 0.92 min (QC Method 2)

### Example 25

### 2-(3-Acetamidopropoxy)-5-chloro-N-(2-oxo-5-(2,4,6-trichlorophenyl)-2,3-dihydro-1H-benzo[e][1,4]diazepin-3-yl)benzamide

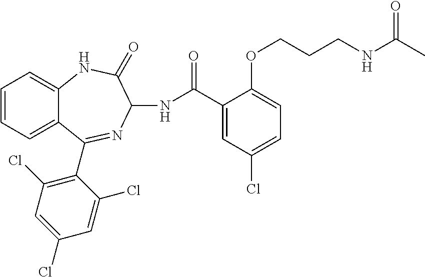

Prepared according to General Method F, using 8b and 28a. Purification by reverse phase chromatography (C18, MeCN:H₂O 7:13→4:1 over 30 min)

NMR δ 11.27 (1H, s), 9.63 (1H, d, J 7.0), 7.93-7.80 (3H, m), 7.73 (1H, d, J 1.9), 7.67-7.53 (2H, m), 7.35-7.11 (4H, m), 5.54 (1H, d, J 7.3), 4.25-4.14 (2H, m), 3.43-3.18 (2H, m), 2.07-1.92 (2H, m), 1.76 (3H, s);

MS (m/e) 609 [M+H]⁺, Rₜ 0.92 min (QC Method 2)

### Example 26

### 5-Chloro-N-(2-oxo-5-(2,4,6-trichlorophenyl)-2,3-dihydro-1H-benzo[e][1,4]diazepin-3-yl)-2-(3-propionamidopropoxy)benzamide

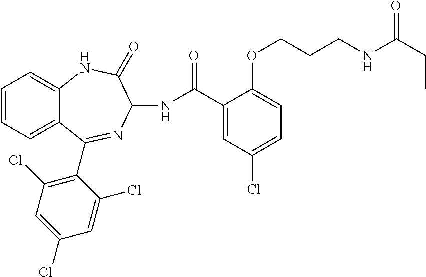

Prepared according to General Method F, using 8b and 28b. Purification by reverse phase chromatography (C18, MeCN:H₂O 7:13→4:1 over 30 min)

NMR δ 11.24 (1H, br s), 9.62 (1H, d, J 7.3), 7.90 (1H, d, J 2.2), 7.88 (1H, d, J 2.8), 7.79-7.70 (2H, m), 7.69-7.57 (2H, m), 7.37-7.16 (4H, m), 5.57 (1H, d, J 7.3), 4.27-4.18 (2H, m), 3.30-3.18 (2H, m), 2.10-1.97 (4H, m), 0.97 (3H, t, J 7.6);

MS (m/e) 623 [M+H]⁺, Rₜ 0.96 min (QC Method 2)

### Example 27

### 5-Chloro-2-(3-(methylsulfonamido)propoxy)-N-(2-oxo-5-(2,4,6-trichlorophenyl)-2,3-dihydro-1H-benzo[e][1,4]diazepin-3-yl)benzamide

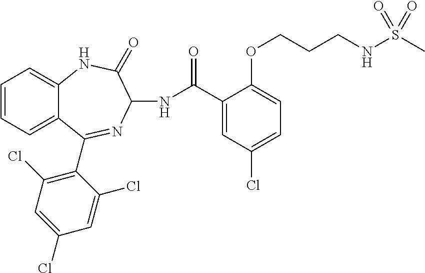

Prepared according to General Method F, using 8b and 28c. Purification by reverse phase chromatography (C18, MeCN:H₂O 7:13→4:1 over 30 min)

NMR δ 11.30 (1H, br s), 9.60 (1H, d, J 7.3), 7.92 (1H, d, J 1.9), 7.86 (1H, d, J 2.8), 7.75 (1H, d, J 1.9), 7.69-7.58 (2H, m), 7.37-7.16 (4H, m), 7.13-7.04 (1H, m), 5.57 (1H, d, J 7.3), 4.33-4.23 (2H, m), 3.27-3.07 (2H, m), 2.82 (3H, s), 2.16-2.02 (2H, m);

MS (m/e) 645 [M+H]⁺, Rₜ 0.96 min (QC Method 2)

### Example 28

### 5-chloro-2-(3-(ethylsulfonamido)propoxy)-N-(2-oxo-5-(2,4,6-trichlorophenyl)-2,3-dihydro-1H-benzo[e][1,4]diazepin-3-yl)benzamide

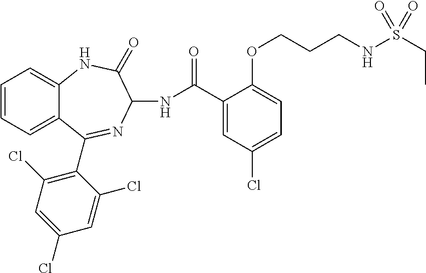

Prepared according to General Method F, using 8b and 28d. Purification by reverse phase chromatography (C18, MeCN:H₂O 7:13→4:1 over 30 min)

NMR δ 11.25 (1H, br s), 9.52 (1H, d, J 7.3), 7.92 (1H, d, J 1.9), 7.85 (1H, d, J 2.8), 7.74 (1H, d, J 1.9), 7.69-7.54 (2H, m), 7.38-7.15 (4H, m), 7.10-6.99 (1H, m), 5.57 (1H, d, J 7.6), 4.34-4.22 (2H, m), 3.38-3.05 (2H, m), 2.89 (2H, q, J 7.3), 2.14-1.98 (2H, m), 1.10 (3H, t, J 7.3);

MS (m/e) 659 [M+H]⁺, Rₜ 0.98 min (QC Method 2)

### Example 29

### 5-Chloro-2-(2-(methylsulfonyl)ethoxy)-N-(2-oxo-5-(2,4,6-trichlorophenyl)-2,3-dihydro-1H-benzo[e][1,4]diazepin-3-yl)benzamide

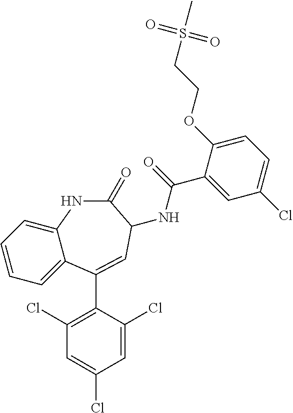

Prepared according to General Method F, using 8b and 31. Purification by column chromatography (SiO₂; PE→EtOAc)

NMR δ 11.21 (1H, br), 9.47 (1H, d, J 7.3), 7.86 (1H, d, J 1.9), 7.78 (1H, d, J 2.8), 7.68 (1H, d, J 1.9), 7.55-7.66 (2H, m), 7.30-7.36 (2H, m), 7.15-7.26 (2H, m), 5.55 (1H, d, J 7.3), 4.58 (2H, t, J 5.7), 3.77 (2H, t, J 5.7), 3.01 (3H, s);

MS (m/e) 614 [M+H]⁺, Rₜ 3.20 min (QC Method 4)

### Example 30

### 5-Fluoro-2-(2-methoxyethoxy)-N-(2-oxo-5-(2,4,6-trichlorophenyl)-2,3-dihydro-1H-benzo[e][1,4]diazepin-3-yl)nicotinamide

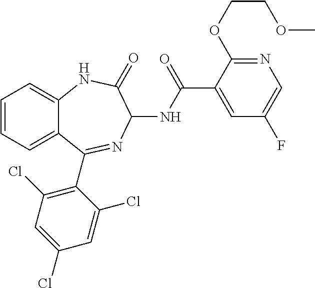

After 30 min, a solution of 32a (2.50 g, 11.6 mmol), HBTU (4.85 g, 12.8 mmol) and TEA (4.04 ml, 29.0 mmol) in DMF (75 ml) was treated with 9 (4.07 g, 11.5 mmol) and the reaction stirred at RT for 72 hrs. The resulting precipitate was collected by filtration and washed with water and ether before being air dried. (Yield 3.95 g) The reaction liquors were concentrated and the residue was taken into DCM, washed with saturated Na₂CO₃ solution, 2M HCl and brine. The organics were dried and concentrated. Purification by column chromatography (SiO₂; DCM→200:8:1 DCM:EtOH:NH₃) gave a second crop of compound (Yield 0.92 g)

Overall yield 4.87 g, 8.85 mmol;

NMR δ 11.28 (1H, br), 9.65 (1H, d, J 7.3), 8.42 (1H, d, J 3.2), 8.17 (1H, dd, J 8.5, 3.2), 7.91 (1H, d, J 1.9), 7.74 (1H, d, J 1.9), 7.65 (1H, m), 7.19-7.35 (3H, m), 5.57 (1H, d, J 7.0), 4.59 (2H, m), 3.83 (2H, m), 3.27 (3H, s);

MS (m/e) 553 [M+H]⁺, Rₜ 3.15 min (QC Method 3)

### Example 31

### 2-(2-Methoxyethoxy)-N-(2-oxo-5-(2,4,6-trichlorophenyl)-2,3-dihydro-1H-benzo[e][1,4]diazepin-3-yl)nicotinamide

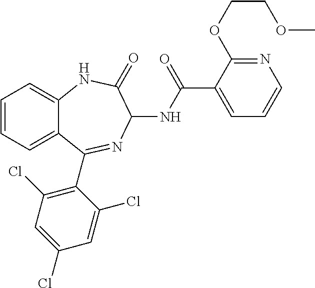

Prepared according to General Method G, using 9 and 32b. Purification by column chromatography (SiO₂; DCM→200:8:1 DCM:EtOH:NH₃)

NMR δ 11.25 (1H, br), 9.58 (1H, d, J 7.3), 8.33-8.41 (2H, m), 7.91 (1H, d, J 1.9), 7.74 (1H, d, J 1.9), 7.65 (1H, m), 7.33 (1H, d, J 7.9), 7.18-7.26 (3H, m), 5.58 (1H, d, J 7.3), 4.62 (2H, m), 3.84 (2H, m), 3.27 (3H, s);

MS (m/e) 535 [M+H]⁺, Rₜ 2.94 min (QC Method 3)

### Example 32

### 5-Chloro-2-(2-methoxyethoxy)-N-(2-oxo-5-(2,4,6-trichlorophenyl)-2,3-dihydro-1H-benzo[e][1,4]diazepin-3-yl)nicotinamide

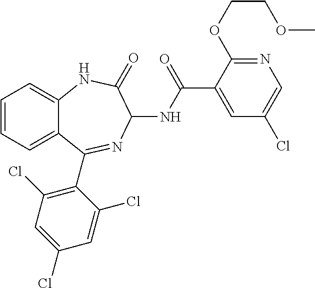

Prepared according to General Method G, using 9 and 32c. Purification by preparative HPLC

NMR δ 11.26 (1H, br), 9.57 (1H, d, J 7.3), 8.43 (1H, d, J 2.9), 8.26 (1H, d, J 2.9), 7.89 (1H, d, J 1.9), 7.72 (1H, d, J 1.9), 7.62 (1H, m), 7.31 (1H, d, J 7.9), 7.16-7.26 (2H, m), 5.54 (1H, d, J 7.0), 4.57 (2H, m), 3.80 (2H, m), 3.25 (3H, s);

MS (m/e) 567 [M+H]⁺, Rₜ 3.54 min (QC Method 4)

### Example 33

### 5-Bromo-2-(2-methoxyethoxy)-N-(2-oxo-5-(2,4,6-trichlorophenyl)-2,3-dihydro-1H-benzo[e][1,4]diazepin-3-yl)nicotinamide

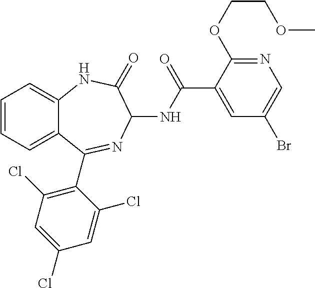

Prepared according to General Method G, using 9 and 32d. Purification by trituration with diethyl ether.

NMR δ 11.25 (1H, br), 9.55 (1H, d, J 7.0), 8.50 (1H, d, J 2.5), 8.36 (1H, d, J 2.5), 7.89 (1H, d, J 1.9), 7.72 (1H, d, J 2.2), 7.62 (1H, m), 7.31 (1H, d, J 7.9), 7.16-7.23 (2H, m), 5.54 (1H, d, J 7.3), 4.57 (2H, m), 3.80 (2H, m), 3.24 (3H, s);

MS (m/e) 613 [M+H]⁺, Rₜ 3.40 min (QC Method 3)

### Example 34

### 2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide

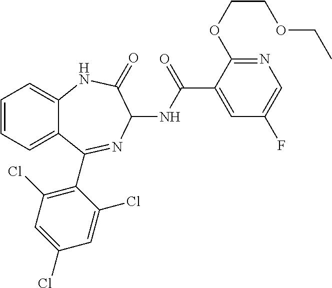

Prepared according to General Method G, using 9 and 32e. Purification by preparative HPLC

NMR δ 11.35 (1H, br), 9.66 (1H, d, J 7.3), 8.48 (1H, d, J 3.2), 8.23 (1H, dd, J 8.5, 3.2), 7.98 (1H, d, J 1.9), 7.82 (1H, d, J 1.9), 7.70 (1H, m), 7.38 (1H, d, J 7.9), 7.23-7.33 (2H, m), 5.63 (1H, d, J 7.3), 4.62 (2H, m), 3.88 (2H, m), 3.49 (2H, q, J 7.0), 1.01 (3H, t, J 7.0);

MS (m/e) 565 [M+H]⁺, Rₜ 3.46 min (QC Method 4)

### Example 35

### 5-Fluoro-2-((S)-2-methoxypropoxy)-N-(2-oxo-5-(2,4,6-trichlorophenyl)-2,3-dihydro-1H-benzo[e][1,4]diazepin-3-yl)nicotinamide

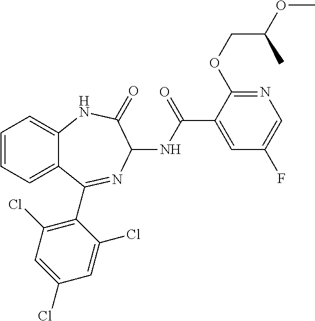

Prepared according to General Method G, using 9 and 32f. Purification by column chromatography (SiO₂; DCM→200:8:1 DCM:EtOH:NH₃)

NMR δ 11.27 (1H, br), 9.58 (0.5H, d, J 7.9), 9.54 (0.5H, d, J 7.7), 8.40 (1H, d, J 3.2), 8.16 (0.5H, dd, J 3.2, 1.3), 8.12 (0.5H, dd, J 3.2, 1.3), 7.91 (1H, m), 7.74 (1H, d, J 1.9), 7.63 (1H, m), 7.31 (1H, d, J 8.2), 7.15-7.26 (2H, m), 5.56 (0.5H, d, J 7.6), 5.55 (0.5H, d, J 7.3), 4.32-4.42 (2H, m), 3.83 (1H, m), 3.20 (3H, d, J 3.2), 1.18 (3H, m);

MS (m/e) 565 [M+H]⁺, Rₜ 3.30 min (QC Method 3)

### Example 36

### 5-Fluoro-2-(2-isopropoxyethoxy)-N-(2-oxo-5-(2,4,6-trichlorophenyl)-2,3-dihydro-1H-benzo[e][1,4]diazepin-3-yl)nicotinamide

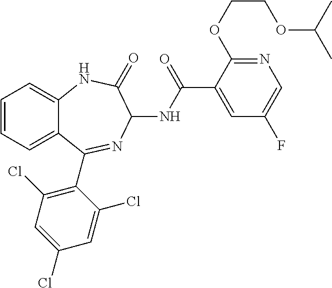

Prepared according to General Method G, using 9 and 32g. Purification by column chromatography (SiO₂; DCM→200:8:1 DCM:EtOH:NH₃)

NMR δ 11.26 (1H, br), 9.52 (1H, d, J 7.6), 8.41 (1H, d, J 3.2), 8.16 (1H, dd, J 8.5, 3.2), 7.90 (1H, d, J 1.9), 7.76 (1H, d, J 1.9), 7.62 (1H, m), 7.30 (1H, d, J 8.2), 7.18-7.25 (2H, m), 5.56 (1H, d, J 7.6), 4.52 (2H, m), 3.78 (2H, m), 3.55 (1H, m), 0.93 (6H, d, J 6.0);

MS (m/e) 579 [M+H]⁺, Rₜ 3.43 min (QC Method 3)

### Example 37

### 5-Fluoro-2-(2-(2-methoxyethoxy)ethoxy)-N-(2-oxo-5-(2,4,6-trichlorophenyl)-2,3-dihydro-1H-benzo[e][1,4]diazepin-3-yl)nicotinamide

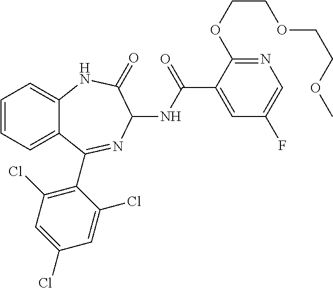

Prepared according to General Method G, using 9 and 32h. Purification by column chromatography (SiO₂; DCM→200:8:1 DCM:EtOH:NH₃), followed by preparative HPLC NMR δ 11.28 (1H, br), 9.60 (1H, d, J 7.6), 8.41 (1H, d, J 3.2), 8.16 (1H, dd, J 8.5, 3.2), 7.91 (1H, d, J 1.9), 7.75 (1H, d, J 1.9), 7.63 (1H, m), 7.30 (1H, d, J 8.2), 7.16-7.26 m), 5.56 (1H, d, J 7.6), 4.55 (2H, m), 3.86 (2H, m), 3.51 (2H, m), 3.26 (2H, m), 3.06 (3H, s);

MS (m/e) 595 [M+H]⁺, Rₜ 3.08 min (QC Method 3)

### Example 38

### 5-Fluoro-2-(3-methoxypropoxy)-N-(2-oxo-5-(2,4,6-trichlorophenyl)-2,3-dihydro-1H-benzo[e][1,4]diazepin-3-yl)nicotinamide

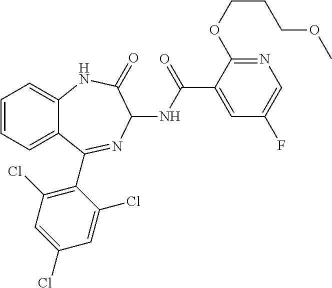

Prepared according to General Method G, using 9 and 32i. Purification by preparative HPLC

NMR δ 11.30 (1H, br), 9.63 (1H, d, J 7.3), 8.40 (1H, d, J 3.2), 8.14 (1H, dd, J 8.6, 3.2), 7.90 (1H, d, J 1.9), 7.73 (1H, d, J 1.9), 7.62 (1H, m), 7.30 (1H, d, J 7.9), 7.15-7.25 (2H, m), 5.54 (1H, d, J 7.3), 4.51 (2H, m), 3.53 (2H, t, J 6.3), 3.17 (3H, s), 2.09 (2H, m);

MS (m/e) 565 [M+H]⁺, Rₜ 3.25 min (QC Method 3)

### Example 39

### 2-(3-Ethoxypropoxy)-5-fluoro-N-(2-oxo-5-(2,4,6-trichlorophenyl)-2,3-dihydro-1H-benzo[e][1,4]diazepin-3-yl)nicotinamide

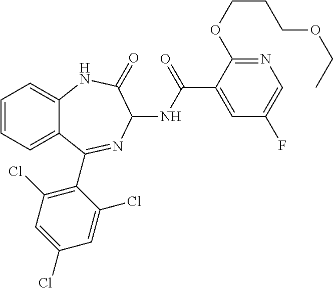

Prepared according to General Method G, using 9 and 32j. Purification by preparative HPLC

NMR δ 11.30 (1H, br), 9.60 (1H, d, J 7.3), 8.40 (1H, d, J 3.2), 8.14 (1H, dd, J 8.5, 3.2), 7.90 (1H, d, J 1.9), 7.73 (1H, d, J 1.9), 7.63 (1H, m), 7.30 (1H, d, J 7.9), 7.15-7.26 (2H, m), 5.54 (1H, d, J 7.3), 4.52 (2H, m), 3.56 (2H, t, J 6.3), 3.33 (2H, q, J 7.3), 2.08 (2H, m), 1.01 (3H, t, J 7.3);

MS (m/e) 579 [M+H]⁺, Rₜ 3.39 min (QC Method 3)

### Example 40

### 2-(2-Methoxyethoxy)-4-methyl-N-(2-oxo-5-(2,4,6-trichlorophenyl)-2,3-dihydro-1H-benzo[e][1,4]diazepin-3-yl)nicotinamide

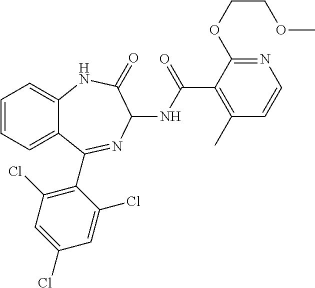

Prepared according to General Method G, using 9 and 32k. Purification by preparative HPLC

NMR δ 11.25 (1H, br), 9.52 (1H, d, J 7.3), 8.22 (1H, d, J 7.6), 7.90 (1H, d, J 1.9), 7.73 (1H, d, J 1.9), 7.62 (1H, m), 7.30 (1H, d, J 7.9), 7.16-7.25 (2H, m), 7.03 (1H, d, J 7.9), 5.53 (1H, d, J 7.3), 4.58 (2H, m), 3.80 (2H, m), 3.24 (3H, s), 2.45 (3H, s);

MS (m/e) 547 [M+H]⁺, Rₜ 3.12 min (QC Method 3)

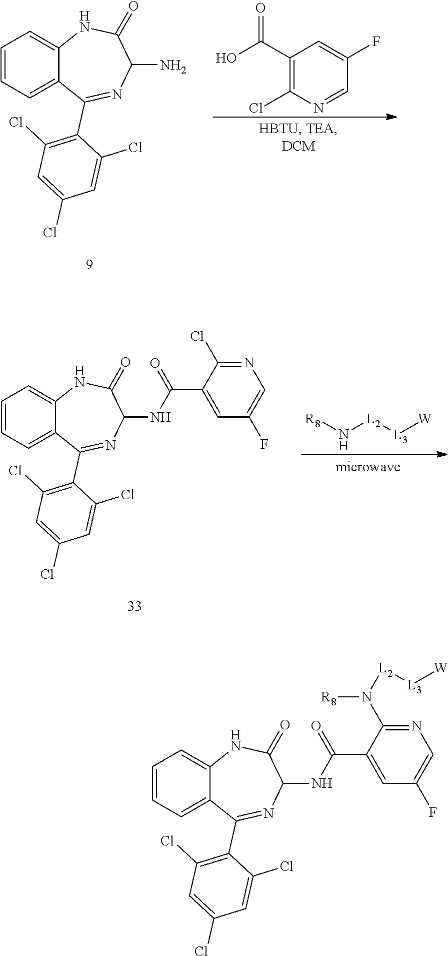

### 2-chloro-5-fluoro-N-(2-oxo-5-(2,4,6-trichlorophenyl)-2,3-dihydro-1H-benzo[e][1,4]diazepin-3-yl)nicotinamide (33)

Prepared according to General Method G, using 9 and 2-chloro-5-fluoronicotinic acid. Material used without purification by chromatography

MS (m/e) 513 [M+H]⁺, Rₜ 2.81 min (QC Method 3)

### General Method H: Microwave Coupled Amino-Nicotinamides

A solution of 33 (200 mg, 0.39 mmol) and amine (340 μl, 3.93 mmol) in 4:1 1,4-dioxane:water (2.5 ml) was heated at 160° C. for 20 min under microwave irradiation. The mixture was concentrated and purified by preparative HPLC, unless otherwise stated.

### Example 41

### 5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide

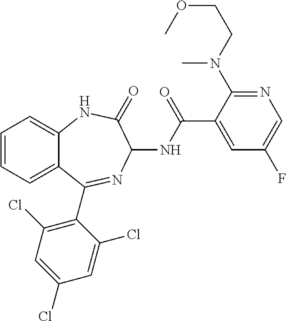

Prepared according to General Method H, using 33 and 2-methoxy-N-methylethanamine

NMR δ 11.19 (1H, br), 9.98 (1H, d, J 8.2), 8.24 (1H, d, J 3.2), 7.93 (1H, d, J 1.9), 7.75 (1H, d, J 1.9), 7.58-7.67 (2H, m), 7.32 (1H, d, J 7.9), 7.14-7.24 (2H, m), 5.52 (1H, d, J 7.9), 3.44-3.54 (4H, m), 3.14 (3H, s), 2.90 (3H, s);

MS (m/e) 564 [M+H]⁺, Rₜ 3.30 min (QC Method 4)

### Example 42

### 5-Fluoro-2-(2-methoxyethylamino)-N-(2-oxo-5-(2,4,6-trichlorophenyl)-2,3-dihydro-1H-benzo[e][1,4]diazepin-3-yl)nicotinamide

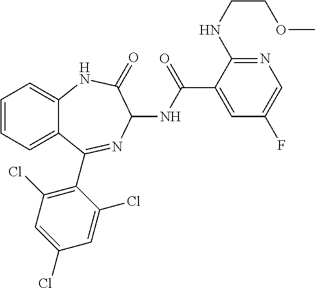

Prepared according to General Method H, using 33 and 2-methoxyethanamine

NMR δ 11.17 (1H, br), 9.78 (1H, d, J 7.6), 8.21-8.33 (3H, m), 7.94 (1H, d, J 1.9), 7.76 (1H, d, J 1.9), 7.64 (1H, m), 7.32 (1H, d, J 7.9), 7.16-7.25 (2H, m), 5.55 (1H, d, J 7.3), 3.40-3.51 (4H, m), 3.22 (3H, s);

MS (m/e) 550 [M+H]⁺, Rₜ 3.23 min (QC Method 4)

### Example 43

### 2-(Ethyl(2-methoxyethyl)amino)-5-fluoro-N-(2-oxo-5-(2,4,6-trichlorophenyl)-2,3-dihydro-1H-benzo[e][1,4]diazepin-3-yl)nicotinamide

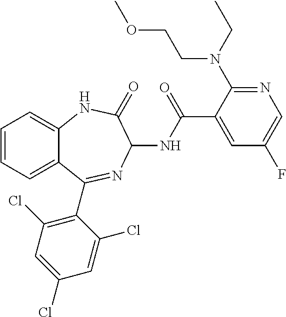

Prepared according to General Method H, using 33 and N-ethyl-2-methoxyethanamine

NMR δ 11.17 (1H, br), 10.44 (1H, d, J 7.9), 8.36 (1H, d, J 3.2), 7.92 (1H, d, J 1.9), 7.78 (1H, dd, J 8.8, 3.2), 7.74 (1H, d, J 1.9), 7.63 (1H, m), 7.31 (1H, d, J 7.9), 7.14-7.25 (2H, m), 5.53 (1H, d, J 7.9), 3.41-3.45 (4H, m), 3.29 (2H, m), 3.09 (3H, s), 0.97 (3H, t, J 7.0);

MS (m/e) 578 [M+H]⁺, Rₜ 3.50 min (QC Method 4)

### Example 44

### 5-Fluoro-2-(isopropyl(2-methoxyethyl)amino)-N-(2-oxo-5-(2,4,6-trichlorophenyl)-2,3-dihydro-1H-benzo[e][1,4]diazepin-3-yl)nicotinamide

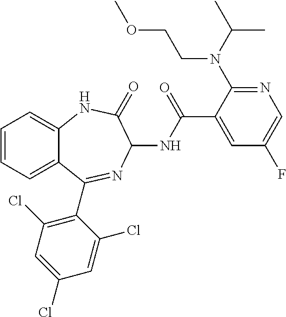

Prepared according to General Method H, using 33 and N-(2-methoxyethyl)propan-2-amine

NMR δ 11.16 (1H, br), 10.70 (1H, d, J 7.3), 8.47 (1H, d, J 3.2), 7.91-7.98 (2H, m), 7.74 (1H, d, J 1.9), 7.62 (1H, m), 7.31 (1H, d, J 8.2), 7.14-7.25 (2H, m), 5.52 (1H, d, J 7.6), 3.48 (2H, m), 3.30-3.37 (3H, m), 3.06 (3H, s), 1.08 (3H, d, J 6.3), 1.00 (3H, d, J 6.3);

MS (m/e) 592 [M+H]⁺, Rₜ 3.71 min (QC Method 4)

### Example 45

### 2-((2-Ethoxyethyl)(ethyl)amino)-5-fluoro-N-(2-oxo-5-(2,4,6-trichlorophenyl)-2,3-dihydro-1H-benzo[e][1,4]diazepin-3-yl)nicotinamide

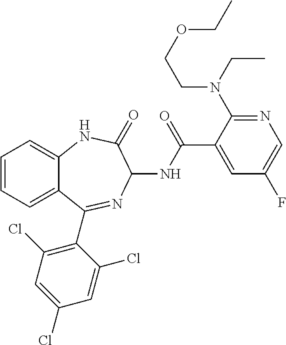

Prepared according to General Method H, using 33 and 2-ethoxy-N-ethylethanamine

NMR δ 11.28 (1H, br), 10.49 (1H, d, J 7.6), 8.37 (1H, d, J 2.8), 7.92 (1H, d, J 1.9), 7.81 (1H, dd, J 8.5, 3.2), 7.74 (1H, d, J 1.9), 7.63 (1H, m), 7.31 (1H, d, J 8.2), 7.14-7.25 (2H, m), 5.53 (1H, d, J 7.9), 3.37-3.48 (4H, m), 3.20-3.32 (4H, m), 0.98 (3H, t, J 7.0), 0.89 (3H, t, J 7.0);

MS (m/e) 592 [M+H]⁺, Rₜ 3.65 min (QC Method 4)

### Example 46

### 2-((2-(Cyclopropylmethoxy)ethyl)(methyl)amino)-5-fluoro-N-(2-oxo-5-(2,4,6-trichlorophenyl)-2,3-dihydro-1H-benzo[e][1,4]diazepin-3-yl)nicotinamide

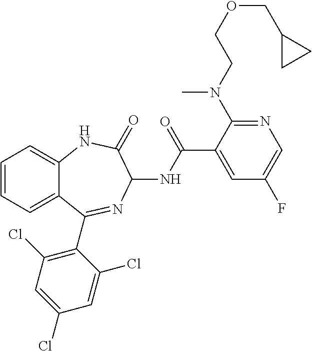

Prepared according to General Method H, using 33 and 2-(cyclopropylmethoxy)-N-methylethanamine

NMR δ 11.15 (1H, br), 9.94 (1H, d, J 7.9), 8.23 (1H, d, J 3.2), 7.91 (1H, d, J 1.9), 7.73 (1H, d, J 1.9), 7.57-7.66 (2H, m), 7.29 (1H, d, J 8.2), 7.12-7.25 (2H, m), 5.51 (1H, d, J 7.9), 3.46-3.54 (4H, m), 3.09 (2H, d, J 6.6), 2.88 (3H, s), 0.84 (1H, m), 0.25-0.33 (2H, m), −0.05-0.03 (2H, m);

MS (m/e) 604 [M+H]⁺, Rₜ 3.62 min (QC Method 4)

### Example 47

### 5-Chloro-N-(5-(2,6-dichlorophenyl)-2-oxo-2,3-dihydro-1H-benzo[e][1,4]diazepin-3-yl)-2-(2-methoxyethoxy)benzamide

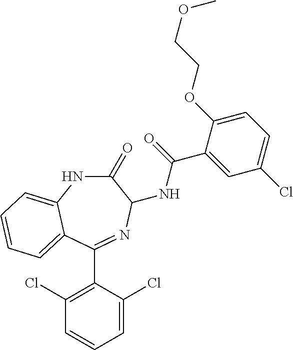

A solution of 8a (250 mg, 0.56 mmol), 21a (260 mg, 1.11 mmol), HBTU (410 mg, 1.11 mmol) and TEA (200 μl, 1.42 mmol) in DMF (3 ml) was stirred for 18hrs. The reaction was concentrated, taken into DCM and washed with saturated Na₂CO₃ and 2M HCl before being dried and re-concentrated.

The residue was taken into anisole (10 ml) and treated with aluminium trichloride (3 g, 22.4 mmol) for 24 hrs before being quenched into saturated NaHCO₃. The resulting slurry was filtered through Celite® and the organics separated and dried. Concentration followed by preparative HPLC gave the title compound

NMR δ 11.25 (1H, s), 9.61 (1H, d, J 7.6), 7.91 (1H, d, J 2.8), 7.59-7.72 (3H, m), 7.49-7.53 (2H, m), 7.16-7.34 (4H, m), 5.58 (1H, d, J 7.6), 4.35 (2H, m), 3.80 (2H, m), 3.22 (3H, s);

MS (m/e) 532 [M+H]⁺, Rₜ 3.03 min (QC Method 3)

### Example 48

### N-(5-(2,6-Dichlorophenyl)-2-oxo-2,3-dihydro-1H-benzo[e][1,4]diazepin-3-yl)-5-fluoro-2-(2-methoxyethoxy)benzamide

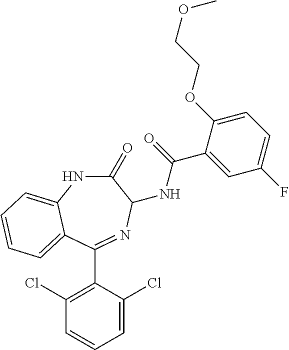

A solution of 8a (250 mg, 0.56 mmol), 21c (240 mg, 1.11 mmol), HBTU (410 mg, 1.11 mmol) and TEA (200 μl, 1.42 mmol) in DMF (3 ml) was stirred for 18 hrs. The reaction was concentrated, taken into DCM and washed with saturated Na₂CO₃ and 2M HCl before being dried and re-concentrated.

The residue was taken into anisole (10 ml) and treated with aluminium trichloride (3 g, 22.4 mmol) for 24 hrs before being quenched into saturated NaHCO₃. The resulting slurry was filtered through Celite® and the organics separated and dried. Concentration followed by preparative HPLC gave the title compound

NMR δ 11.24 (1H, s), 9.67 (1H, d, J 7.6), 7.65-7.72 (2H, m), 7.59-7.65 (1H, m), 7.49-7.53 (2H, m), 7.39-7.49 (1H, m), 7.15-7.35 (4H, m), 5.59 (1H, d, J 7.3), 4.34 (2H, m), 3.81 (2H, m), 3.22 (3H, s);

MS (m/e) 516 [M+H]⁺, Rₜ 2.78 min (QC Method 3)

### Example 49

### 5-Chloro-N-(5-(2,4-dichloro-6-(morpholinomethyl)phenyl)-2-oxo-2,3-dihydro-1H-benzo[e][1,4]diazepin-3-yl)-2-(2-methoxyethoxy)benzamide

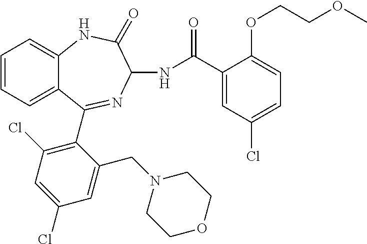

Prepared according to General Method G, using 17a and 21a. Purification by column chromatography (C18, MeCN:H₂O 0:1→4:1 over 30 min)

>20:1 mixture of diastereoisomers

NMR δ 11.21 (1H, s), 9.66 (1H, d, J 7.2), 7.91 (1H, d, J 2.8), 7.65-7.54 (4H, m), 7.34-7.14 (4H, m), 5.50 (1H, d, J 7.2), 4.36 (2H, t, J 4.4), 4.14 (1H, d, J 13.0), 3.93-3.76 (2H, m), 3.55-3.37 (4H, m), 3.26 (3H, s), 3.22 (1H, d, J 13.0), 2.53-2.36 (4H, m);

MS (m/e) 631 [M+H]⁺, Rₜ 2.38 min (QC Method 4)

### Example 50

### N-(5-(2,4-Dichloro-6-(morpholinomethyl)phenyl)-2-oxo-2,3-dihydro-1H-benzo[e][1,4]diazepin-3-yl)-5-fluoro-2-(2-methoxyethoxy)benzamide

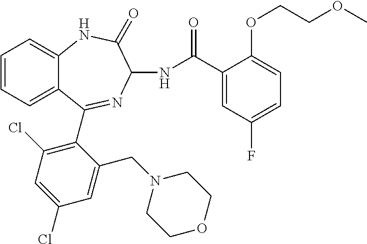

Prepared according to General Method G, using 17a and 21c. Purification by column chromatography (C18, MeCN:H₂O 0:1→4:1 over 30 min)

>20:1 mixture of diastereoisomers

NMR δ 11.20 (1H, br s), 9.72 (1H, d, J 7.1), 7.73-7.54 (4H, m), 7.49-7.38 (1H, m), 7.36-7.14 (4H, m), 5.50 (1H, d, J 7.1), 4.35 (2H, t, J 4.5), 4.13 (1H, d, J 13.0), 3.93-3.75 (2H, m), 3.55-3.32 (4H, m), 3.26 (3H, s), 3.23 (1H, d, J 13.0), 2.53-2.35 (4H, m);

MS (m/e) 615 [M+H]⁺, Rₜ 2.22 min (QC Method 4)

### Example 51

### 5-Chloro-N-(5-(2,4-dichloro-6-(morpholinomethyl)phenyl)-2-oxo-2,3-dihydro-1H-benzo[e][1,4]diazepin-3-yl)-2-(2-methoxyethoxy)nicotinamide

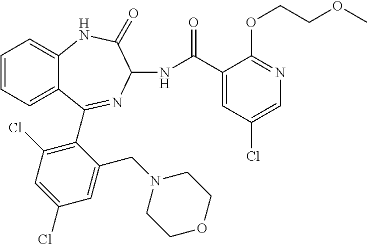

Prepared according to General Method G, using 17a and 32c. Purification by column chromatography (C18, MeCN:H₂O 0:1→4:1 over 30 min)

NMR δ 11.22 (1H, s), 9.64 (1H, d, J 7.0), 8.44 (1H, d, J 2.8), 8.25 (1H, d, J 2.8), 7.63-7.50 (3H, m), 7.30-7.13 (3H, m), 5.46 (1H, d, J 7.0), 4.61-4.53 (2H, m), 4.12 (1H, d, J 13.0), 3.85-3.78 (2H, m), 3.53-3.32 (4H, m), 3.26 (3H, s), 3.19 (1H, d, J 13.0), 2.51-2.33 (4H, m);

MS (m/e) 632 [M+H]⁺, Rₜ 3.56 min (QC Method 5)

### Example 52

### N-(5-(2,4-Dichloro-6-(morpholinomethyl)phenyl)-2-oxo-2,3-dihydro-1H-benzo[e][1,4]diazepin-3-yl)-5-fluoro-2-(2-methoxyethoxy)nicotinamide

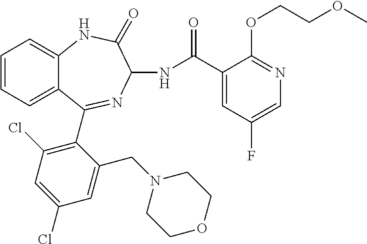

Prepared according to General Method G, using 17a and 32a. Purification by column chromatography (C18, MeCN:H₂O 0:1→4:1 over 30 min)

NMR δ 11.28 (0.3H, s), 11.22 (0.7H, s), 9.69 (0.7H, d, J 7.1), 9.61 (0.3H, d, J 7.1), 8.39 (1H, d, J 3.2), 8.19-8.09 (1H, m), 7.75 (0.3H, d, J 1.9), 7.64-7.51 (2.3H, m), 7.42 (0.3H, d, J 1.9), 7.33-7.09 (3.1H, m), 5.53 (0.3H, d, J 7.1), 5.46 (0.7H, d, J 7.1), 4.60-4.51 (2H, m), 4.11 (0.6H, d, J 13.0), 3.85-3.75 (2H, m), 3.52-3.13 (4.7H, m), 3.26 (2H, s), 3.22 (1H, s), 3.00-2.83 (0.7H, m), 2.55-2.33 (2.8H, m), 2.01-1.76 (1.2H, m); MS (m/e) 616 [M+H]⁺, Rₜ 4.48, 4.54 min (QC Method 5)

### Example 53

### N-(5-(2,4-Dichloro-6-methoxyphenyl)-2-oxo-2,3-dihydro-1H-benzo[e][1,4]diazepin-3-yl)-5-fluoro-2-(2-methoxyethoxy)benzamide

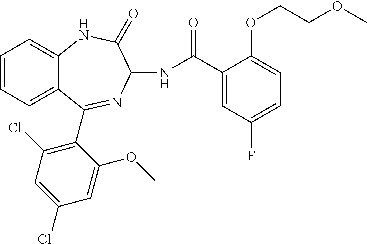

Prepared according to General Method G, using 17b and 21c. Purification by column chromatography (C18, MeCN:H₂O 0:1→4:1 over 30 min)

NMR δ 11.11 (0.6H, s), 11.10 (0.4H, s), 9.59 (1H, d, J 7.6), 7.69-7.52 (2H, m), 3.91 (1.8H, s), (7H, m), 5.49 (0.6H, d, J 7.6), 5.47 (0.4H, d, J 7.6), 4.35-4.26 (2H, m), 3.91 (1.8H, s), 3.83-3.71 (2H, m), 3.52 (1.2H, s), 3.21 (1.8H, s), 3.18 (1.2H, s);

MS (m/e) 546 [M+H]⁺, Rₜ 2.97 min (QC Method 3)

### Example 54

### N-(5-(2,4-Dichloro-6-methoxyphenyl)-2-oxo-2,3-dihydro-1H-benzo[e][1,4]diazepin-3-yl)-5-fluoro-2-(2-methoxyethoxy)nicotinamide

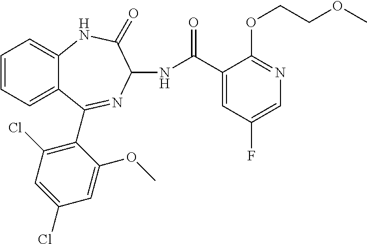

Prepared according to General Method G, using 17b and 32a. Purification by column chromatography (C18, MeCN:H₂O 0:1→4:1 over 30 min)

NMR δ 11.16 (1H, br s), 9.58 (0.4H, d, J 7.2), 9.57 (0.6H, d, J 7.2), 8.39 (1H, d, J 3.2), 8.17-8.10 (1H, m), 7.62-7.53 (1H, m), 7.37-7.14 (5H, m), 5.47 (0.6H, d, J 7.2), 5.46 (0.4H, d, J 7.2), 4.61-4.50 (2H, m), 3.90 (1.8H, s), 3.83-3.74 (2H, m), 3.52 (1.2H, s), 3.25 (1.2H, s), 3.22 (1.8H, s);

MS (m/e) 547 [M+H]⁺, Rₜ 2.97 min (QC Method 3)

### Example 55

### N-(5-(2,4-Dichloro-6-methoxyphenyl)-2-oxo-2,3-dihydro-1H-benzo[e][1,4]diazepin-3-yl)-2-(2-ethoxyethoxy)-5-fluoronicotinamide

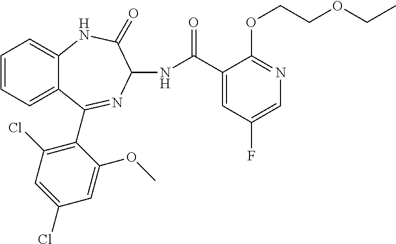

Prepared according to General Method G, using 17b and 32e. Purification by column chromatography (C18, MeCN:H₂O 0:1→4:1 over 30 min)

NMR δ 11.16 (0.6H, br s), 11.15 (0.4H, br s), 9.53 (0.4H, d, J 7.5), 9.50 (0.6H, d, J 7.5), 8.40 (1H, d, J 3.2), 8.18-8.10 (1H, m), 7.62-7.53 (1H, m), 7.35 (1H, dd, J 11.5, 8.2), 7.30-7.12 (4H, m), 5.49 (0.6H, d, J 7.5), 5.48 (0.4H, d, J 7.5), 4.62-4.46 (2H, m), 3.90 (1.8H, s), 3.85-3.74 (2H, m), 3.52 (1.2H, s), 3.42 (1.2H, q, J 7.0), 3.41 (0.8H, q, J 7.0), 0/94 (1.2H, t, J 7.0), 0.93 (1.8H, t, J 7.0);

MS (m/e) 561 [M+H]⁺, Rₜ 3.12 min (QC Method 3)

### Example 56

### N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide

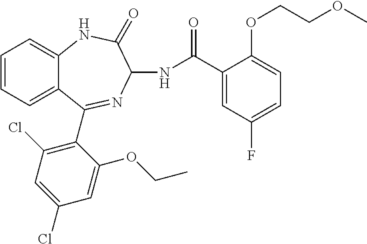

Prepared according to General Method G, using 17c and 21c. Purification by column chromatography (C18, MeCN:H₂O 0:1→4:1 over 30 min)

NMR δ 11.15 (0.6H, s), 11.12 (0.4H, s), 9.61 (0.4H, d, J 7.5), 9.60 (0.6H, d, J 7.5), 7.70-7.61 (1H, m), 7.61-7.52 (1H, m), 7.46-7.35 (1H, m), 7.35-7.08 (6H, m), 5.50 (0.4H, d, J 7.5), 5.49 (0.6H, d, J 7.5), 4.37-4.04 (3H, m), 3.92-3.58 (3H, m), 3.21 (3H, s), 1.27 (1.2H, t, J 7.0), 0.82 (1.8H, t, J 7.0);

MS (m/e) 560 [M+H]⁺, Rₜ 3.14 min (QC Method 3)

### Example 57

### N-(5-(2,4-Dichloro-6-ethoxyphenyl)-2-oxo-2,3-dihydro-1H-benzo[e][1,4]diazepin-3-yl)-5-fluoro-2-(2-methoxyethoxy)nicotinamide

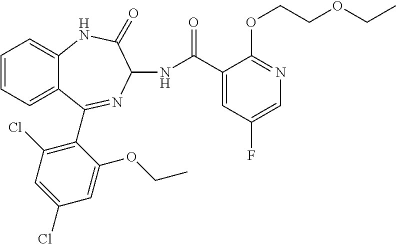

Prepared according to General Method G, using 17c and 32a. Purification by column chromatography (C18, MeCN:H₂O 0:1→4:1 over 30 min)

NMR δ 11.20 (0.6H, s), 11.17 (0.4H, s), 9.60 (0.4H, d, J 7.3), 9.59 (0.6H, d, J 7.3), 8.39 (1H, d, J 3.2), 8.18-8.09 (1H, m), 7.61-7.52 (1H, m), 7.35-7.09 (5H, m), 5.48 (0.4H, d, J 7.3), 5.47 (0.6H, d, J 7.3), 4.61-4.51 (2H, m), 4.28-4.05 (0.6H, m), 3.92-3.57 (3.4H, m), 3.25 (1.8H, s), 3.24 (1.2H, s), 1.28 (1.2H, t, J 7.0), 0.82 (1.8H, t, J 7.0);

MS (m/e) 561 [M+H]⁺, Rₜ 3.16 min (QC Method 3)

### Example 58

### N-(5-(2,4-Dichloro-6-ethoxyphenyl)-2-oxo-2,3-dihydro-1H-benzo[e][1,4]diazepin-3-yl)-2-(2-ethoxyethoxy)-5-fluoronicotinamide

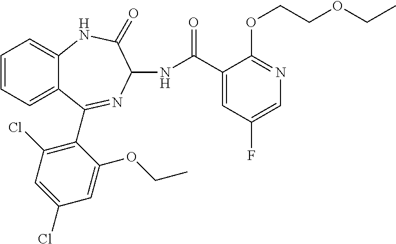

Prepared according to General Method G, using 17c and 32e. Purification by column chromatography (C18, MeCN:H₂O 0:1→4:1 over 30 min)

NMR δ 11.18 (0.6H, s), 11.16 (0.4H, s), 9.54 (1H, d, J 7.5), 8.40 (1H, d, J 3.2), 8.14 (1H, ddd, J 8.5, 5.1, 3.2), 7.61-7.52 (1H, m), 7.34-7.09 (5H, m), 5.49 (1H, d, J 7.5), 4.63-4.46 (2H, m), 4.28-4.06 (0.7H, m), 3.92-3.59 (3.3H, m), 3.43 (2H, q, J 7.0), 1.26 (1.2H, t, J 7.0), 0.96 (1.2H, t, J 7.0), 0.94 (1.8H, t, J 7.0), 0.84 (1.8H, t, J 7.0);

MS (m/e) 575 [M+H]⁺, Rₜ 3.30 min (QC Method 3)

### Example 59

### (S)-5-Chloro-2-(2-methoxyethoxy)-N-(2-oxo-5-(2,4,6-trichlorophenyl)-2,3-dihydro-1H-benzo[e][1,4]diazepin-3-yl)benzamide

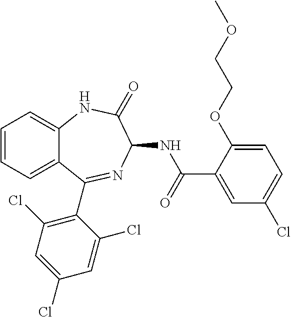

Prepared from Example 14, using preparative HPLC:

Column: 250×50 mm CHIRALPAK® IC 5 μm

Mobile phase: Dichloromethane

Flow rate: 180 ml/min

Detection: 325 nm

Temperature: 21° C.

NMR δ 11.26 (1H, s), 9.61 (1H, d, J 7.3), 7.93 (1H, d, J 1.9), 7.90 (1H, d, J 2.8), 7.75 (1H, d, J 1.9), 7.58-7.69 (2H, m), 7.18-7.36 (4H, m), 5.58 (1H, d, J 7.3), 4.36 (2H, m), 3.81 (2H, m), 3.23 (3H, s);

MS (m/e) 568 [M+H]⁺, Rₜ 3.30 min (QC Method 3)

### Example 60

### (R)-5-Chloro-2-(2-methoxyethoxy)-N-(2-oxo-5-(2,4,6-trichlorophenyl)-2,3-dihydro-1H-benzo[e][1,4]diazepin-3-yl)benzamide

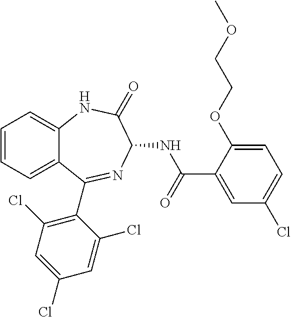

Prepared from Example 14, using preparative HPLC:

Column: 250×50 mm CHIRALPAK® IC 5 μm

Mobile phase: Dichloromethane

Flow rate: 180 ml/min

Detection: 325 nm

Temperature: 21° C.

NMR δ 11.26 (1H, s), 9.61 (1H, d, J 7.3), 7.93 (1H, d, J 1.9), 7.90 (1H, d, J 2.8), 7.75 (1H, d, J 1.9), 7.58-7.69 (2H, m), 7.18-7.36 (4H, m), 5.58 (1H, d, J 7.3), 4.36 (2H, m), 3.81 (2H, m), 3.23 (3H, s);

MS (m/e) 568 [M+H]⁺, Rₜ 3.30 min (QC Method 3)

### Example 61

### (S)-5-Fluoro-2-(2-methoxyethoxy)-N-(2-oxo-5-(2,4,6-trichlorophenyl)-2,3-dihydro-1H-benzo[e][1,4]diazepin-3-yl)benzamide

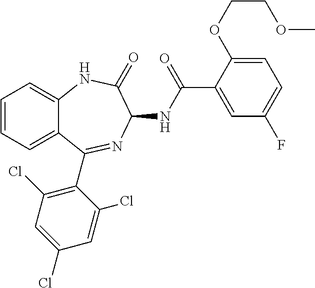

Prepared from Example 16, using preparative HPLC:

Column: 250×50 mm CHIRALPAK® IA 5 μm

Mobile phase: 40/60/1 n-Heptane/Dichloromethane/Ethanol

Flow rate: 120 ml/min

Detection: UV 250 nm

Temperature: 25° C.

NMR δ 11.24 (1H, br, s), 9.66 (1H, d, J 7.3), 7.91 (1H, d, J 1.9), 7.74 (1H, d, J 1.9), 7.59-7.70 (2H, m), 7.42 (1H, m), 7.16-7.34 (4H, m), 5.56 (1H, d, J 7.6), 4.32 (2H, m), 3.79 (2H, m), 3.23 (3H, s);

MS (m/e) 550 [M+H]⁺, Rₜ 1.07 min (QC Method 2)

### Example 62

### (R)-5-Fluoro-2-(2-methoxyethoxy)-N-(2-oxo-5-(2,4,6-trichlorophenyl)-2,3-dihydro-1H-benzo[e][1,4]diazepin-3-yl)benzamide

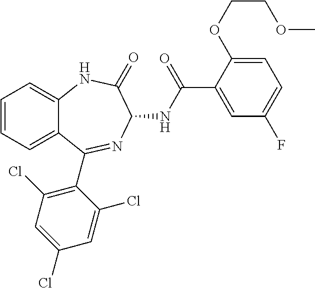

Prepared from Example 16, using preparative HPLC:

Column: 250×50 mm CHIRALPAK® IA 5 μm

Mobile phase: 40/60/1 n-Heptane/Dichloromethane/Ethanol

Flow rate: 120 ml/min

Detection: UV 250 nm

Temperature: 25° C.

NMR δ 11.24 (1H, br, s), 9.66 (1H, d, J 7.3), 7.91 (1H, d, J 1.9), 7.74 (1H, d, J 1.9), 7.59-7.70 (2H, m), 7.42 (1H, m), 7.16-7.34 (4H, m), 5.56 (1H, d, J 7.6), 4.32 (2H, m), 3.79 (2H, m), 3.23 (3H, s);

MS (m/e) 550 [M+H]⁺, Rₜ 1.07 min (QC Method 2)

### Example 63

### (S)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide

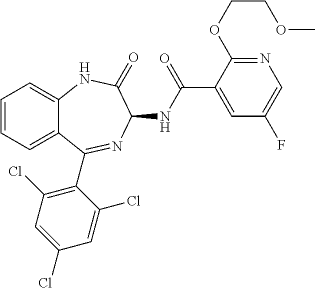

Prepared from Example 30, using preparative HPLC:

Column: CHIRALCEL® OZ 20 μm-250×76 mm

Mobile phase: Acetonitrile

Flow rate: 270 ml/min

Detection: UV 260 nm

Temperature: 40° C.

NMR δ 11.28 (1H, br), 9.65 (1H, d, J 7.3), 8.42 (1H, d, J 3.2), 8.17 (1H, dd, J 8.5, 3.2), 7.91 (1H, d, J 1.9), 7.74 (1H, d, J 1.9), 7.65 (1H, m), 7.19-7.35 (3H, m), 5.57 (1H, d, J 7.0), 4.59 (2H, m), 3.83 (2H, m), 3.27 (3H, s);

MS (m/e) 553 [M+H]⁺, Rₜ 3.15 min (QC Method 3)

### Example 64

### (R)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide

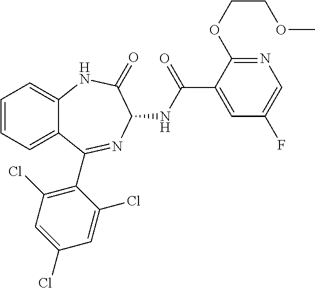

Prepared from Example 30, using preparative HPLC:

Column: CHIRALCEL® OZ 20 μm-250×76 mm

Mobile phase: Acetonitrile

Flow rate: 270 ml/min

Detection: UV 260 nm

Temperature: 40° C.

NMR δ 11.28 (1H, br), 9.65 (1H, d, J 7.3), 8.42 (1H, d, J 3.2), 8.17 (1H, dd, J 8.5, 3.2), 7.91 (1H, d, J 1.9), 7.74 (1H, d, J 1.9), 7.65 (1H, m), 7.19-7.35 (3H, m), 5.57 (1H, d, J 7.0), 4.59 (2H, m), 3.83 (2H, m), 3.27 (3H, s);

MS (m/e) 553 [M+H]⁺, Rₜ 3.15 min (QC Method 3)

### Example 65

### (S)-2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide

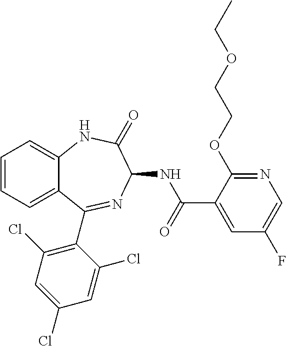

Prepared from Example 34, using preparative HPLC:

Column: 250×50 mm CHIRALPAK® IC 5 μM

Mobile phase: n-Heptane/Dichloromethane/Ethanol 50/50/1

Flow rate: 120 ml/min

Detection: UV 250 nm

Temperature: 25° C.

NMR δ 11.35 (1H, br), 9.66 (1H, d, J 7.3), 8.48 (1H, d, J 3.2), 8.23 (1H, dd, J 8.5, 3.2), 7.98 (1H, d, J 1.9), 7.82 (1H, d, J 1.9), 7.70 (1H, m), 7.38 (1H, d, J 7.9), 7.23-7.33 (2H, m), 5.63 (1H, d, J 7.3), 4.62 (2H, m), 3.88 (2H, m), 3.49 (2H, q, J 7.0), 1.01 (3H, t, J 7.0);

MS (m/e) 565 [M+H]⁺, Rₜ 3.46 min (QC Method 4)

### Example 66

### (R)-2-(2-Ethoxyethoxy)-5-fluoro-N-(2-oxo-5-(2,4,6-trichlorophenyl)-2,3-dihydro-1H-benzo[e][1,4]diazepin-3-yl)nicotinamide

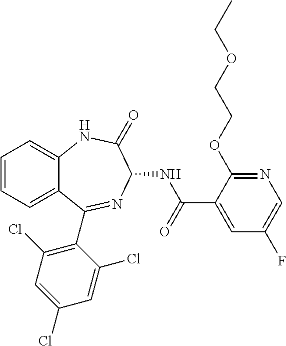

Prepared from Example 34, using preparative HPLC:

Column: 250×50 mm CHIRALPAK® IC 5 μM

Mobile phase: n-Heptane/Dichloromethane/Ethanol 50/50/1

Flow rate: 120 ml/min

Detection: UV 250 nm

Temperature: 25° C.

NMR δ 11.35 (1H, br), 9.66 (1H, d, J 7.3), 8.48 (1H, d, J 3.2), 8.23 (1H, dd, J 8.5, 3.2), 7.98 (1H, d, J 1.9), 7.82 (1H, d, J 1.9), 7.70 (1H, m), 7.38 (1H, d, J 7.9), 7.23-7.33 (2H, m), 5.63 (1H, d, J 7.3), 4.62 (2H, m), 3.88 (2H, m), 3.49 (2H, q, J 7.0), 1.01 (3H, t, J 7.0);

MS (m/e) 565 [M+H]⁺, Rₜ 3.46 min (QC Method 4)

### Example 67

### (S)-5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide

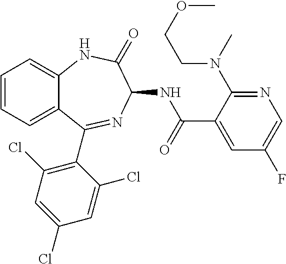

Prepared from Example 41, using preparative HPLC:

Column: 250×10 mm CHIRALPAK® IA 5 μm

Mobile phase: 80% Ethanol in i-hexane

Flow rate: 1.0 ml/min

Detection: UV 254 nm

Temperature: 20° C.

NMR δ 11.19 (1H, br), 9.98 (1H, d, J 8.2), 8.24 (1H, d, J 3.2), 7.93 (1H, d, J 1.9), 7.75 (1H, d, J 1.9), 7.58-7.67 (2H, m), 7.32 (1H, d, J 7.9), 7.14-7.24 (2H, m), 5.52 (1H, d, J 7.9), 3.44-3.54 (4H, m), 3.14 (3H, s), 2.90 (3H, s);

MS (m/e) 564 [M+H]⁺, Rₜ 3.30 min (QC Method 4)

### Example 68

### (R)-5-Fluoro-2-((2-methoxyethyl)(methyl)amino)-N-(2-oxo-5-(2,4,6-trichlorophenyl)-2,3-dihydro-1H-benzo[e][1,4]diazepin-3-yl)nicotinamide

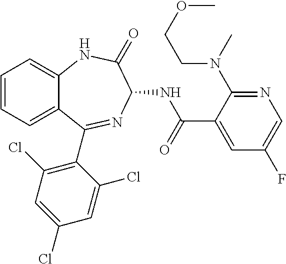

Prepared from Example 41, using preparative HPLC:

Column: 250×10 mm CHIRALPAK® IA 5 μm

Mobile phase: 80% Ethanol in i-hexane

Flow rate: 1.0 ml/min

Detection: UV 254 nm

Temperature: 20° C.

NMR δ 11.19 (1H, br), 9.98 (1H, d, J 8.2), 8.24 (1H, d, J 3.2), 7.93 (1H, d, J 1.9), 7.75 (1H, d, J 1.9), 7.58-7.67 (2H, m), 7.32 (1H, d, J 7.9), 7.14-7.24 (2H, m), 5.52 (1H, d, J 7.9), 3.44-3.54 (4H, m), 3.14 (3H, s), 2.90 (3H, s);

MS (m/e) 564 [M+H]⁺, Rₜ 3.30 min (QC Method 4)

### Example 69

### (S)—N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide

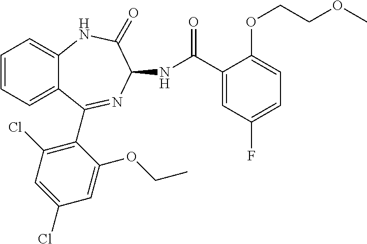

Prepared from Example 56, using preparative HPLC:

Column: 250×10 mm CHIRALPAK® IA 5 μm

Mobile phase: 5% Ethanol in 60% Dichloromethane/i-hexane

Flow rate: 2.0 ml/min

Detection: UV 254 nm

Temperature: 20° C.

NMR δ 11.15 (0.6H, s), 11.12 (0.4H, s), 9.61 (0.4H, d, J 7.5), 9.60 (0.6H, d, J 7.5), 7.70-7.61 (1H, m), 7.61-7.52 (1H, m), 7.46-7.35 (1H, m), 7.35-7.08 (6H, m), 5.50 (0.4H, d, J 7.5), 5.49 (0.6H, d, J 7.5), 4.37-4.04 (3H, m), 3.92-3.58 (3H, m), 3.21 (3H, s), 1.27 (1.2H, t, J 7.0), 0.82 (1.8H, t, J 7.0);

MS (m/e) 560 [M+H]⁺, Rₜ 3.31 min (QC Method 4)

### Example 70

### (R)—N-(5-(2,4-Dichloro-6-ethoxyphenyl)-2-oxo-2,3-dihydro-1H-benzo[e][1,4]diazepin-3-yl)-5-fluoro-2-(2-methoxyethoxy)benzamide

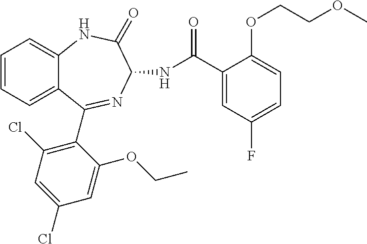

Prepared from Example 56, using preparative HPLC:

Column: 250×10 mm CHIRALPAK® IA 5 μm

Mobile phase: 5% Ethanol in 60% Dichloromethane/i-hexane

Flow rate: 2.0 ml/min

Detection: UV 254 nm

Temperature: 20° C.

NMR δ 11.15 (0.6H, s), 11.12 (0.4H, s), 9.61 (0.4H, d, J 7.5), 9.60 (0.6H, d, J 7.5), 7.70-7.61 (1H, m), 7.61-7.52 (1H, m), 7.46-7.35 (1H, m), 7.35-7.08 (6H, m), 5.50 (0.4H, d, J 7.5), 5.49 (0.6H, d, J 7.5), 4.37-4.04 (3H, m), 3.92-3.58 (3H, m), 3.21 (3H, s), 1.27 (1.2H, t, J 7.0), 0.82 (1.8H, t, J 7.0);

MS (m/e) 560 [M+H]⁺, Rₜ 3.31 min (QC Method 4)

1. A compound of Formula (I), or a pharmaceutically acceptable salt thereof: 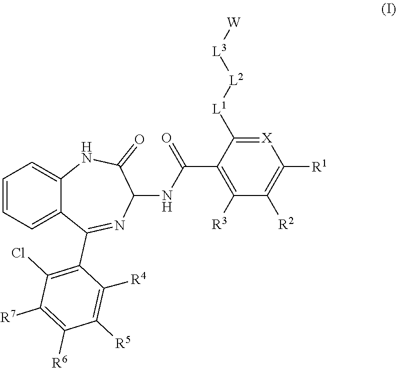
wherein:
L¹ represents O or NR⁸, wherein R⁸ represents hydrogen, C₁₋₃alkyl, acetyl, trifluoromethyl or trifluoromethylcarbonyl;
L² represents C₁₋₆alkylene;
L³ represents O, NR⁹ or S(O)ₙ, wherein R⁹ represents hydrogen or C₁₋₃alkyl and n represents 0, 1 or 2;
W represents
C₁₋₆alkyl, C₁₋₃alkylaminoC₁₋₆alkyl, diC₁₋₃alkylaminoC₁₋₆alkyl, C₁₋₆alkoxyC₁₋₆alkyl, C₂₋₄alkanoyl, C₁₋₄alkylsulfonyl, C₁₋₄alkylaminocarbonyl, diC₁₋₄alkylaminocarbonyl, haloC₁₋₃alkyl or C₃₋₆cycloalkylC₁₋₃alkyl;
aryl, wherein said aryl ring is optionally substituted with 1, 2 or 3 substituents selected from R¹⁰;
a 5 or 6 membered monocyclic heteroaryl ring which comprises 1, 2, 3 or 4 heteroatoms independently selected from O, N or S, wherein said heteroaryl ring is optionally substituted with 1, 2 or 3 substituents selected from R¹⁰; or
W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
X represents CH or N;
R¹ represents hydrogen or fluoro;
R² represents hydrogen, halo, C₁₋₃alkyl, C₁₋₃alkoxy, formyl, C₂₋₃alkanoyl, trifluoromethyl or trifluoromethoxy;
R³ represents hydrogen, C₁₋₃alkyl or halo;
R⁴ represents hydrogen, halo, C₁₋₃alkyl, C₁₋₃alkoxy, haloC₁₋₃alkyl, C₁₋₆alkoxyC₁₋₆alkyl or —(CH₂)ₚ—NR¹¹R¹², wherein p represents 1 or 2 and R¹¹ and R¹² independently represent hydrogen or C₁₋₃alkyl, or R¹¹ and R¹² are joined so as to form a 4, 5, 6 or 7 membered heterocyclic ring which optionally comprises, in addition to the nitrogen atom to which R¹¹ and R¹² are attached, 1 or 2 further heteroatoms independently selected from O, N or S, and wherein said heterocyclic ring is optionally substituted with 1, 2 or 3 substituents selected from R¹⁰;
R⁵ represents hydrogen, C₁₋₃alkyl or halo;
R⁶ represents hydrogen, halo, C₁₋₃alkyl, C₁₋₃alkoxy, haloC₁₋₃alkyl or haloC₁₋₃alkoxy;
R⁷ represents hydrogen, C₁₋₃alkyl, C₁₋₃alkoxy, halo, trifluoromethyl or trifluoromethoxy; and
R¹⁰ represents C₁₋₃alkyl or halo.

2. A compound according to claim 1, or a pharmaceutically acceptable salt thereof, wherein:
L¹ represents O or NR⁸, wherein R⁸ represents hydrogen, C₁₋₃alky, acetyl, trifluoromethyl or trifluoromethylcarbonyl;
L² represents C₁₋₆alkylene;
L³ represents O, NR⁹ or S(O)ₙ, wherein R⁹ represents hydrogen or C₁₋₃alkyl and n represent 0, 1 or 2;
W represents
C₁₋₆alkyl, diC₁₋₃alkylamino C₁₋₆alkyl, C₁₋₆alkoxyC₁₋₆alkyl, C₂₋₄alkanoyl, C₁₋₄alkylsulfonyl, C₁₋₄alkylaminocarbonyl, haloC₁₋₃alkyl or C₃₋₆cycloalkylC₁₋₃alkyl;
aryl, wherein said aryl ring is optionally substituted with 1, 2 or 3 substituents selected from R¹⁰;
a 5 or 6 membered monocyclic heteroaryl ring which comprises 1, 2, 3 or 4 heteroatoms independently selected from O, N or S, wherein said heteroaryl ring is optionally substituted with 1, 2 or 3 substituents selected from R¹⁰; or
W and L² are joined so as to form a 4, 5, 6 or 7 membered heterocyclic ring which comprises L³ and optionally comprises, in addition to L³, 1 or 2 further heteroatoms independently selected from O, N or S, wherein said heterocyclic ring is optionally substituted with 1, 2 or 3 substituents selected from R¹⁰;
X represents CH or N;
R¹ represents hydrogen or fluoro;
R² represents hydrogen, halo, C₁₋₃alkyl, C₁₋₃alkoxy, formyl, C₂₋₃alkanoyl or trifluoromethyl, trifluoromethoxy;
R³ represents hydrogen, C₁₋₃alkyl or halo;
R⁴ represents hydrogen, halo, C₁₋₃alkyl, C₁₋₃alkoxy, haloC₁₋₃alkyl, C₁₋₆alkoxyC₁₋₆alkyl or —(CH₂)ₚ—NR¹¹R¹², wherein p represents 1 or 2 and R¹¹ and R¹² independently represent hydrogen or C₁₋₃alkyl, or R¹¹ and R¹² are joined so as to form a 4, 5, 6 or 7 membered heterocyclic ring which optionally comprises, in addition to the nitrogen atom to which R¹¹ and R¹² are attached, 1 or 2 further heteroatoms independently selected from O, N or S, and wherein said heterocyclic ring is optionally substituted with 1, 2 or 3 substituents selected from R¹⁰;
R⁵ represents hydrogen, C₁₋₃alkyl or halo;
R⁶ represents hydrogen, halo, C₁₋₃alkyl, C₁₋₃alkoxy, haloC₁₋₃alkyl or haloC₁₋₃alkoxy;
R⁷ represents hydrogen, C₁₋₃alkyl, C₁₋₃alkoxy, halo, trifluoromethyl or trifluoromethoxy; and
R¹⁰ represents C₁₋₃alkyl or halo.

3. A compound according to claim 1, or a pharmaceutically acceptable salt thereof, wherein the compound of Formula (I) has the configuration shown in Formula (IA):

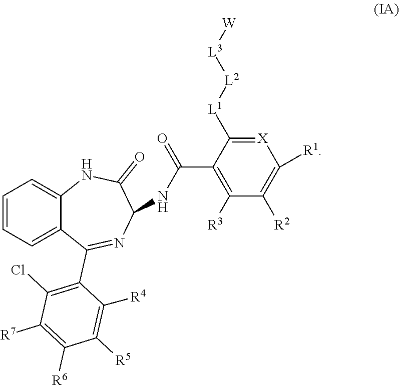

4. A compound according to claim 1, or a pharmaceutically acceptable salt thereof, wherein L¹ represents O.

5. A compound according to claim 1, or a pharmaceutically acceptable salt thereof, wherein L² represents ethylene.

6. A compound according to claim 1, or a pharmaceutically acceptable salt thereof, wherein L³ represents O.

7. A compound according to claim 1, or a pharmaceutically acceptable salt thereof, wherein W represents C₁₋₆alkyl.

8. A compound according to claim 1, or a pharmaceutically acceptable salt thereof, wherein W represents methyl or ethyl.

9. A compound according to claim 1, or a pharmaceutically acceptable salt thereof, wherein X represents CH.

10. A compound according to claim 1, or a pharmaceutically acceptable salt thereof, wherein X represents N.

11. A compound according to claim 1, or a pharmaceutically acceptable salt thereof, wherein R¹ and R³ represent hydrogen and R² represents halo.

12. (canceled)

13. A compound according to claim 1, or a pharmaceutically acceptable salt thereof, wherein R⁴ represents halo or C₁₋₆alkoxy.

14. A compound according to claim 1, or a pharmaceutically acceptable salt thereof, wherein R⁴ represents Cl, methoxy or ethoxy.

15. A compound according to claim 1, or a pharmaceutically acceptable salt thereof, wherein R⁵ and R⁷ represent hydrogen and R⁶ represents Cl.

16. A compound according to claim 1 which is selected from Examples 1 to 70 or a pharmaceutically acceptable salt thereof.

17. (S)-5-Fluoro-2-(2-methoxyethoxy)-N-(2-oxo-5-(2,4,6-trichlorophenyl)-2,3-dihydro-1H-benzo[e][1,4]diazepin-3-yl)nicotinamide or a pharmaceutically acceptable salts thereof.

18. A pharmaceutical composition which comprises a compound according to claim 1, or a pharmaceutically acceptable salt thereof, in association with a pharmaceutically acceptable diluent or carrier.

19. A method of treating, or reducing the risk of, hepatitis C virus infection in a warm-blooded animal, such as man, in need of such treatment which comprises administering to said animal an effective amount of a compound according to claim 1, or a pharmaceutically acceptable salt thereof.

20. A process for the preparation of a compound of Formula (I) as defined hereinbefore, or a pharmaceutically acceptable salt thereof, which comprises a process (a), (b) or (c) wherein, unless otherwise defined, the variables are as defined in claim 1 for compounds of Formula (I):
(a) reaction of a compound of Formula (II), or a salt thereof, with a compound of Formula (III) in the presence of a suitable coupling agent and a suitable base: 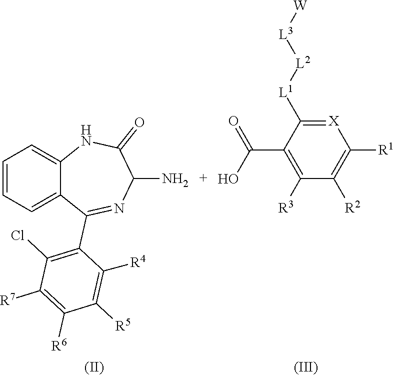
(b) reaction of a compound of Formula (IV) with a compound of Formula (III) in the presence of a suitable coupling agent and a suitable base, wherein P¹ represents a suitable protecting group: 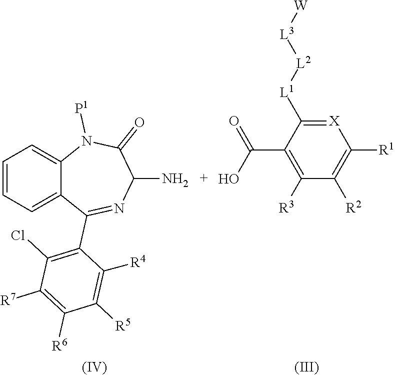
(c) when L¹ represents NR⁸, reaction of a compound of Formula (V) with an amine of Formula (VI) wherein Y represents a suitable leaving group: 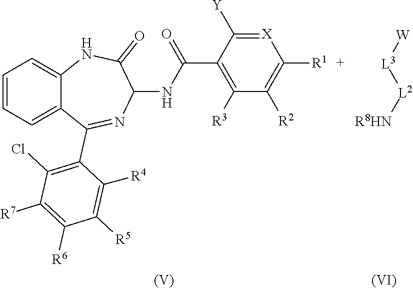
and thereafter, if necessary:
(i) converting a compound of Formula (I) into another compound of Formula (I);
(ii) removing any protecting groups;
(iii) separating a racemic mixture into separate enantiomers; and/or
(iv) preparing a pharmaceutically acceptable salt thereof

21. A combination product which comprises a compound according to claim 1, or a pharmaceutically acceptable salt thereof, and one or more further active ingredients that are selected from a HCV protease inhibitor, a HCV polymerase inhibitor, a HCV helicase inhibitor, an interferon, ribavirin and a HCV NS5a inhibitor.

22. (canceled)
